(19) United States
(12) Patent Application Publication
(10) Pub. No.: US 2021/0032661 A1
(43) Pub. Date: Feb. 4, 2021

(54) Methods And Compositions Comprising A Viral Vector For Expression Of A Transgene And An Effector

(71) Applicant: THE TRUSTEES OF THE UNIVERSITY OF PENNSYLVANIA, Philadelphia, PA (US)

(72) Inventors: Daniel J. Powell, Bala Cynwyd, PA (US); Anze Smole, Philadelphia, PA (US); Avery D. Posey, Philadelphia, PA (US); Donald O'Rourke, Wynnewood, PA (US); Yibo Yin, Philadelphia, PA (US); Carl June, Merion Station, PA (US); Philipp Romel, Philadelphia, PA (US)

(21) Appl. No.: 17/046,253

(22) PCT Filed: Apr. 8, 2019
(86) PCT No.: PCT/US2019/026378

§ 371 Date: Oct. 8, 2020

Related U.S. Application Data
(60) Provisional application No. 62/655,048, filed on Apr. 9, 2018.

(51) Int. Cl. C12N 15/86; C07K 14/725; C07K 14/715; A61K 35/17; C07K 16/32; C07K 14/705; C07K 14/54

(52) U.S. Cl. CPC C12N 15/86; C07K 14/7051; C07K 14/7155; A61K 35/17; C07K 16/32; C07K 14/70521; C07K 2317/76; C07K 14/5434; C12N 2740/15043; C12N 2830/002; C07K 2319/33; C07K 2319/60; C07K 2317/622; C07K 14/70578

(57) ABSTRACT

The present invention relates to compositions and methods comprising a single viral vector comprising both a first polynucleotide comprising a constitutive promoter operably linked to a nucleic acid encoding at least one transgene, wherein one of the at least one transgenes encodes a receptor or receptor subunit, a receptor fusion protein or a fluorescent marker; and a second polynucleotide comprising an inducible promoter operably linked to a nucleic acid encoding an effector. Also provided are engineered cells comprising the viral vector and methods for generating the engineered cells comprising the viral vector. Also provided is site-specific integration of the genetic element into the a gene locus by means of a CRISPR-related system. Further provided are methods for treating a patient having a disease, a disorder or condition associated with expression of an antigen, the method comprising administering to the patient an effective amount of a composition comprising the engineered cell.

### CROSS-REFERENCE TO RELATED APPLICATION

This application claims priority to U.S. Provisional Application No. 62/655,048, filed Apr. 9, 2018, which is hereby incorporated by reference in its entirety.

### BACKGROUND OF THE INVENTION

Immunotherapy has demonstrated extraordinary clinical responses in treating various blood cancers which recently led to the first FDA-approved chimeric antigen receptor T cell (CAR T) therapy for patients up to 25 years of age with relapsed or refractory acute lymphoblastic leukemia (ALL).

CAR T therapy has thus far demonstrated limited success in the solid tumor setting. This is attributed to various immune cell intrinsic features, such as T cell exhaustion, as well as active cancer immunosuppressive mechanisms, hostile tumor microenvironment, presence of various immunosuppressive cells (e.g., regulatory T cells (T_{REGS}), myeloid-derived suppressor cells (MDSCs)) and apparent lack of unique surface antigens. (Johnson, L. A. & June, C. H. Driving gene-engineered T cell immunotherapy of cancer. Cell Res. 27, 38-58 (2016)).

Adoptive cell immunotherapy (ACT) sometimes induces serious treatment-related complications such as cytokine release syndrome (CRS) and neurological toxicities, which need to be addressed to advance the field. (June, C. H., Warshauer, J. T. & Bluestone, J. A. Is autoimmunity the Achilles' heel of cancer immunotherapy? Nat. Med. 23, 540-547 (2017); Neelapu, S. S. et al. Chimeric antigen receptor T-cell therapy—assessment and management of toxicities. Nat. Rev. Clin. Oncol. (2017). doi:10.1038/nrclinonc.2017.148) Current strategies for treating such complications that include systemic administration of Tocilizumab that blocks the IL-6 signaling axis and/or corticosteroids often fail to reliably control adverse events associated with immunotherapy. This is in part because the drug is generally administered after CRS has presented with clinically observable symptoms, at which point it may already have caused considerable damage to the patient. In the case of neurological toxicities, there is an added complication: large molecule biologics (e.g., recombinant antibodies like Tocilizumab) administered systemically (e.g., intravenously) cannot pass the blood-brain barrier, and so must be administered directly to the brain, a more time-consuming and invasive procedure. Taken together, early detection and treatment are needed to prevent damage while controlling neurological toxicities is inefficient because blood-brain barrier prevents entry of the drug into the CNS (Johnson, L. A. & June, C. H. Driving gene-engineered T cell immunotherapy of cancer. Cell Res. 27, 38-58 (2016)).

There remains an unmet need for methods and compositions for engineering immune cells by introducing genetic circuits that would endow engineered immune cells with additional functionalities, such as autonomous antigen-induced expression of immunomodulatory molecules limited to the tumor microenvironment.

### SUMMARY

Provided is a single viral vector comprising: a first polynucleotide comprising a constitutive promoter operably linked to a nucleic acid encoding at least one transgene, wherein one of the at least one transgenes encodes a receptor or receptor subunit, a receptor fusion protein or a fluorescent marker; and a second polynucleotide comprising an inducible promoter operably linked to a nucleic acid encoding an effector. In some embodiments, the transgene encodes a receptor fusion protein. In some embodiments, the receptor fusion protein comprises an antigen-specific synNotch receptor and an optional reporter. In some embodiments, the receptor fusion protein further comprises a 2A peptide between the antigen-specific synNotch receptor and the reporter.

In some embodiments, the transgene encodes a receptor or receptor subunit. In some embodiments, the receptor or receptor subunit is a TCR or TCR subunit. In further embodiments, the receptor is a TCR, wherein the TCR comprises an extracellular antigen binding domain capable of specifically binding a cognate antigen and an intracellular domain. In yet further embodiments, the intracellular domain drives expression of the effector when the antigen-binding domain of the TCR binds its cognate antigen by activating the inducible promoter operably linked to the effector.

In some embodiments, the receptor or receptor subunit is a cytokine receptor or cytokine receptor subunit. In some embodiments, the cytokine receptor subunit is IL-6Ra.

In some embodiments, the receptor fusion protein is a chimeric antigen receptor (CAR) comprising an extracellular antigen-binding domain capable of specifically binding a cognate antigen and an intracellular signaling domain, wherein the inducible promoter is capable of driving expression of the effector when the antigen binding domain binds its target.

In some embodiments, the receptor fusion protein is a CAR comprising an extracellular antigen-binding domain capable of specifically binding a cognate antigen and an intracellular signaling domain. In further embodiments, the intracellular signaling domain drives expression of the effector when the antigen-binding domain of the CAR binds its cognate antigen by activating the inducible promoter operably linked to the effector. In yet further embodiments, the inducible promoter is capable of driving expression of the effector when the receptor binds its ligand.

In some embodiments, the transgene encodes a fluorescent marker. In further embodiments, the fluorescent marker is mCherry.

In some embodiments, the first polynucleotide, the second polynucleotide or both the first and the second polynucleotides comprise an insulator nucleic acid sequence or a linker nucleic acid sequence.

In some embodiments, the constitutive promoter comprises a promoter selected from the group consisting of an EF-1alpha promoter, a PGK-1 promoter, an UBC promoter, a CMV promoter, a CAGG promoter and an SV40 promoter.

In some embodiments, the inducible promoter comprises a promoter selected from the group consisting of an NFAT promoter, a STAT3-sensing promoter, a CD69 promoter, a CD137 promoter or a hypoxia responsive element promoter and a minimal promoter operably linked to an inducible enhancer. In some embodiments, the inducible promoter is linked to a minimal promoter (P_{MIN}).

In some embodiments, the effector is a cytokine, an interferon, a chemokine, an antibody or antibody fragment, a bispecific antibody, a BiTE, a checkpoint inhibitor antagonist, an enzyme, a regulatory element, dCas9, a transcription factor or a DNA binding domain of a transcription factor.

In some embodiments, the receptor fusion protein comprises a transcription factor and a transcription activator. In further embodiments, the transcription activator is VP64.

In some embodiments, the effector is a fusion protein comprising at least two components, wherein each component is selected from the group consisting of a cytokine, an interferon, a chemokine, an antibody or antibody fragment, a bispecific antibody, a BiTE, a checkpoint inhibitor antagonist, an enzyme, a regulatory element, a transcription factor or a DNA binding domain of a transcription factor, wherein the at least two components are linked through a 2A peptide.

In some embodiments, the BiTE binds a surface antigen on a target cell. In some embodiments the BiTE binds wild type or variant III EGFR. In some embodiments, the BiTE further binds CD3.

In some embodiments, the cytokine is IL-2, IL-12, IL-15, IL-18 or IL-21. In some embodiments, the interferon is interferon alpha or interferon beta. In some embodiments, the antibody or antibody fragment is anti-IL-6, anti-IL-6R, anti-IL-6Ra, anti-TNFalpha, anti-IL-1, anti-PD1, anti-CD40, anti-CD25, anti-CD3, anti-CD20, anti-IL-8, anti-MCP1, anti-MIP-1, anti-TGFβ, anti-CD47, anti-CSF1R, anti-CD28, anti-TIGIT, anti-VEGFR or anti-FAP. In further embodiments, the antibody or antibody fragment is agonistic.

In some embodiments, the checkpoint inhibitor antagonist is anti-PD-L1, anti-PD-1, anti-CTLA4, anti-LAG3, anti-TIM3, anti-2B4 or anti-CD160 or anti-CD5. In some embodiments, the bispecific antibody comprises an antigen binding domain of at least one of anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-CD40 antagonistic antibody, anti-IL-8, anti-MCP1, anti-MIP-1, anti-TGFβ, anti-CD47, anti-CSF1R, anti-CD28, anti-TIGIT, anti-VEGFR, anti-FAP, anti-PD-L1, anti-PD-1, anti-CTLA4, anti-LAG3, anti-TIM3, anti-2B4, anti-CD160 or anti-CD5, or combinations thereof. In some embodiments, the enzyme is heparinase, collagenase or a metalloproteinase.

In some embodiments, the chemokine is CCL5 (RANTES), XCL-1, XCL-2, CCR-7, CCL-19 or CCL-21. In some embodiments, the regulatory element, transcription factor or portion thereof is T-bet, TCF7, EOMES, a Runx family member, BLIMP1, Bc12, Bc16, FoxP3, FoxO1, or FoxO1-3A, or a portion thereof. In further embodiments, the Runx family member is Runx1 or Runx3.

In some embodiments, the viral vector is a retroviral vector, an adenoviral vector or an adeno-associated viral vector. In further embodiments, the retroviral vector is a lentiviral vector.

In some embodiments, the inducible promoter and the constitutive promoter drive transcription in the same direction, or in different directions.

In some embodiments, the CAR comprises an antigen binding domain, a transmembrane domain, and an intracellular signaling domain. In further embodiments, the intracellular signaling domain comprises one or more costimulatory signaling domains and an immunoreceptor tyrosine activation motif-containing signaling domain. In yet further embodiments, the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83. In some embodiments, the immunoreceptor tyrosine activation motif-containing signaling domain comprises a CD3 zeta signaling domain.

Provided is an engineered cell comprising the viral vector of any one of the previous embodiments. In some embodiments, the engineered cell is an immune cell or precursor cell thereof. In some embodiments, the engineered cell is an NK cell, a B cell, a dendritic cell or a macrophage. In some embodiments, an endogenous TCR of the engineered cell has been knocked out using a CRISPR-related system, a TALEN or a Zinc finger nuclease.

Provided is a method for generating an engineered cell, comprising introducing the viral vector of any one of the previous embodiments into a cell. In some embodiments, the engineered cell is an immune cell or a precursor of an immune cell. In some embodiments, the method further comprises knocking out at least one endogenous TCR subunit of the engineered cell. In further embodiments, the at least one endogenous TCR subunit is knocked out using a CRISPR-related system, a TALEN or a Zinc finger nuclease. In yet further embodiments, the CRISPR-related system is CRISPR/Cas9. In some embodiments, both subunits of the endogenous TCR are knocked out.

Provided is a method for treating a patient having a disease, disorder or condition associated with expression of an antigen, the method comprising administering to the patient an effective amount of a composition comprising the engineered cell of any one of the previous embodiments. In some embodiments, the expression of the antigen is abnormal. In some embodiment, the engineered cell is an immune cell or precursor cell thereof. In further embodiments, the engineered cell is an NK cell, a B cell, a dendritic cell or a macrophage. In some embodiments, the composition further comprises a pharmaceutically acceptable carrier or adjuvant.

Provided is an engineered immune cell or engineered immune cell precursor cell, comprising: an insertion in a gene locus, wherein the insertion comprises a first polynucleotide comprising a constitutive promoter operably linked to a nucleic acid encoding at least one transgene, wherein one of the at least one transgenes encodes a receptor or receptor subunit, a receptor fusion protein or a fluorescent marker; and a second polynucleotide comprising an inducible promoter operably linked to a nucleic acid encoding an effector. In some embodiments, the transgene encodes a receptor fusion protein. In some embodiments, the receptor fusion protein comprises an antigen-specific synNotch receptor and an optional reporter.

In some embodiments, the transgene encodes a receptor or receptor subunit. In some embodiments, the receptor or receptor subunit is a TCR or TCR subunit. In further embodiments, the receptor is a TCR, wherein the TCR comprises an extracellular antigen binding domain capable of specifically binding a cognate antigen and an intracellular domain. In yet further embodiments, the intracellular domain drives expression of the effector when the antigen-binding domain of the TCR binds its cognate antigen by activating the inducible promoter operably linked to the effector.

In some embodiments, the receptor or receptor subunit is a cytokine receptor or cytokine receptor subunit. In some embodiments, the cytokine receptor subunit is IL-6Ra.

In some embodiments, the receptor fusion protein is a chimeric antigen receptor (CAR) comprising an extracellular antigen-binding domain capable of specifically binding a cognate antigen and an intracellular signaling domain, wherein the inducible promoter is capable of driving expression of the effector when the antigen binding domain binds its target.

In some embodiments, the receptor fusion protein is a chimeric antigen receptor (CAR) comprising an extracellular antigen-binding domain capable of specifically binding a cognate antigen and an intracellular signaling domain. In further embodiments, the intracellular signaling domain drives expression of the effector when the antigen-binding domain of the CAR binds its cognate antigen by activating the inducible promoter operably linked to the effector. In yet further embodiments, the inducible promoter is capable of driving expression of the effector when the receptor binds its ligand.

In some embodiments, the transgene encodes a fluorescent marker. In further embodiments, the fluorescent marker is mCherry.

In some embodiments, the first polynucleotide, the second polynucleotide or both the first and the second polynucleotides comprise an insulator nucleic acid sequence or a linker nucleic acid sequence.

In some embodiments, the insertion is encoded in a donor template. In further embodiments, the donor template is encoded in a viral vector. In some embodiments, the gene locus encodes a TCR subunit, an HLA or an immune checkpoint molecule. In further embodiments, the immune checkpoint molecule is PD-1, TIM3, LAG3, CTLA4, 2B4, CD160 or CD5.

In some embodiments, the insertion in a gene locus is mediated by a CRISPR-related system. In further embodiments, the CRISPR-related system is CRISPR/Cas9.

In some embodiments, the constitutive promoter comprises a promoter selected from the group consisting of an EF-1alpha promoter, a PGK-1 promoter, an UBC promoter, a CMV promoter, a CAGG promoter and an SV40 promoter.

In some embodiments, the inducible promoter comprises a promoter selected from the group consisting of an NFAT promoter, a STAT3-sensing promoter, a CD69 promoter, a CD137 promoter or a hypoxia responsive element promoter and a minimal promoter operably linked to an inducible enhancer. In some embodiments, the inducible promoter is linked to a minimal promoter (P_{MIN}).

In some embodiments, the effector is a cytokine, an interferon, a chemokine, an antibody or antibody fragment, a bispecific antibody, a BiTE, a checkpoint inhibitor antagonist, an enzyme, a regulatory element, dCas9, a transcription factor or a DNA binding domain of a transcription factor.

In some embodiments, the receptor fusion protein comprises a transcription factor and a transcription activator. In further embodiments, the transcription activator is VP64.

In some embodiments, the effector is a fusion protein comprising at least two components, wherein each component is selected from the group consisting of a cytokine, an interferon, a chemokine, an antibody or antibody fragment, a bispecific antibody, a BiTE, a checkpoint inhibitor antagonist, an enzyme, a regulatory element, a transcription factor or a DNA binding domain of a transcription factor, wherein the at least two components are linked through a 2A peptide.

In some embodiments, the BiTE binds a surface antigen on a target cell. In some embodiments the BiTE binds wild type or variant III EGFR. In some embodiments, the BiTE further binds CD3.

In some embodiments, the cytokine is IL-2, IL-12, IL-15, IL-18 or IL-21. In some embodiments, the interferon is interferon alpha or interferon beta. In some embodiments, the antibody or antibody fragment is anti-IL-6, anti-IL-6R, anti-IL-6Ra, anti-TNFalpha, anti-IL-1, anti-PD1, anti-CD40, anti-CD25, anti-CD3, anti-CD20, anti-IL-8, anti-MCP1, anti-MIP-1, anti-TGFβ, anti-CD47, anti-CSF1R, anti-CD28, anti-TIGIT, anti-VEGFR or anti-FAP. In further embodiments, the antibody or antibody fragment is agonistic.

In some embodiments, the checkpoint inhibitor antagonist is anti-PD-L1, anti-PD-1, anti-CTLA4, anti-LAG3, anti-TIM3, anti-2B4 or anti-CD160 or anti-CD5. In some embodiments, the bispecific antibody comprises an antigen binding domain of at least one of anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-CD40 antagonistic antibody, anti-IL-8, anti-MCP1, anti-MIP-1, anti-TGFβ, anti-CD47, anti-CSF1R, anti-CD28, anti-TIGIT, anti-VEGFR, anti-FAP, anti-PD-L1, anti-PD-1, anti-CTLA4, anti-LAG3, anti-TIM3, anti-2B4, anti-CD160 or anti-CD5, or combinations thereof. In some embodiments, the enzyme is heparinase, collagenase or a metalloproteinase.

In some embodiments, the chemokine is CCL5 (RANTES), XCL-1, XCL-2, CCR-7, CCL-19 or CCL-21. In some embodiments, the regulatory element, transcription factor or portion thereof is T-bet, TCF7, EOMES, a Runx family member, BLIMP1, Bc12, Bc16, FoxP3, FoxO1, or FoxO1-3A, or a portion thereof. In further embodiments, the Runx family member is Runx1 or Runx3.

In some embodiments, the viral vector is a retroviral vector, an adenoviral vector or an adeno-associated viral vector. In further embodiments, the retroviral vector is a lentiviral vector.

In some embodiments, the inducible promoter and the constitutive promoter drive transcription in the same direction, or in different directions.

In some embodiments, the CAR comprises an antigen binding domain, a transmembrane domain, and an intracellular signaling domain. In further embodiments, the intracellular signaling domain comprises one or more costimulatory signaling domains and an immunoreceptor tyrosine activation motif-containing signaling domain. In yet further embodiments, the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83. In some embodiments, the immunoreceptor tyrosine activation motif-containing signaling domain comprises a CD3 zeta signaling domain.

In some embodiments, the engineered cell is an NK cell, a B cell, a dendritic cell or a macrophage, or a precursor of an NK cell, a precursor of a B cell, a precursor of a dendritic cell or a precursor of a macrophage.

Provided is a method for treating a patient having a disease, disorder or condition associated with expression of an antigen, the method comprising administering to the patient an effective amount of a composition comprising the engineered cell of any one of the previous embodiments. In some embodiments, the expression of the antigen is abnormal.

Provided is a viral vector comprising: a first polynucleotide comprising a first constitutive promoter and a nucleic acid encoding at least one transgene, wherein the first constitutive promoter is operably linked to the nucleic acid encoding the at least one transgene, wherein one of the at least one transgenes is a receptor, a receptor fusion protein or a fluorescent marker; and a second polynucleotide comprising a second constitutive promoter and a nucleic acid encoding an effector, wherein the second constitutive promoter is operably linked to the nucleic acid encoding the effector. In some embodiments, the transgene encodes a receptor fusion protein. In some embodiments, the transgene encodes a receptor or receptor subunit. In further embodiments, the receptor or receptor subunit is a TCR or TCR subunit. In yet further embodiments, the receptor is a TCR, wherein the TCR comprises an extracellular antigen binding domain capable of specifically binding a cognate antigen and an intracellular domain.

In some embodiments, the receptor or receptor subunit is a cytokine receptor or a cytokine receptor subunit. In further embodiments, the cytokine receptor subunit is IL-6Ra.

In some embodiments, the receptor fusion protein is a CAR comprising an extracellular antigen-binding domain capable of specifically binding a cognate antigen and an intracellular signaling domain.

In some embodiments, the transgene encodes a fluorescent marker. In further embodiments, the fluorescent marker is mCherry.

In some embodiments, the first polynucleotide, the second polynucleotide or both the first and the second polynucleotides comprise an insulator sequence or a linker sequence.

In some embodiments, the first and/or second constitutive promoter is an EF-1alpha promoter, a PGK-1 promoter, an UBC promoter, a CMV promoter, a CAGG promoter or an SV40 promoter.

In some embodiments, the effector is a cytokine, an interferon, a chemokine, an antibody or antibody fragment, a bispecific antibody, a checkpoint inhibitor antagonist, an enzyme, a regulatory element, dCas9, a transcription factor or a DNA binding domain of a transcription factor.

Also provided is an engineered immune cell comprising the vector of any one of the preceding embodiments.

Provided is an engineered immune cell or engineered immune cell precursor cell, comprising: an insertion in a gene locus, wherein the insertion comprises a first polynucleotide comprising a first constitutive promoter and a nucleic acid encoding at least one transgene, wherein the first constitutive promoter is operably linked to the nucleic acid encoding the at least one transgene, wherein one of the at least one transgenes encodes a receptor or receptor subunit, a receptor fusion protein or a fluorescent marker; and a second polynucleotide comprising a second constitutive promoter and a nucleic acid encoding an effector, wherein the second constitutive promoter is operably linked to the nucleic acid encoding the effector. In some embodiments, the transgene encodes a receptor fusion protein. In some embodiments, the transgene encodes a receptor or receptor subunit.

In some embodiments, the effector is a cytokine, an interferon, a chemokine, an antibody or antibody fragment, a bispecific antibody, a checkpoint inhibitor antagonist, an enzyme, a regulatory element, dCas9, a transcription factor or a DNA binding domain of a transcription factor.

Also provided is a viral vector comprising: a polynucleotide comprising a constitutive promoter, a nucleic acid encoding a first transgene, and a nucleic acid encoding a second transgene, wherein the constitutive promoter is operably linked to the nucleic acid encoding the first transgene and the second transgene; wherein the first transgene is a receptor or receptor subunit, a receptor fusion protein or a fluorescent marker; and wherein the second transgene is an effector. In some embodiments, the receptor fusion protein is a CAR. In some embodiments, the effector is a transcription factor.

Also provided is an engineered immune cell or engineered immune cell precursor cell, comprising: an insertion in a gene locus, wherein the insertion comprises a polynucleotide comprising a constitutive promoter, a nucleic acid encoding a first transgene, and a nucleic acid encoding a second transgene, wherein the constitutive promoter is operably linked to the nucleic acid encoding the first transgene and the second transgene; wherein the first transgene is a receptor or receptor subunit, a receptor fusion protein or a fluorescent marker; and wherein the second transgene is an effector. In some embodiments, the receptor fusion protein is a CAR. In some embodiments, the effector is a transcription factor.

In some embodiments, the CAR comprises an antigen binding domain, a transmembrane domain, and an intracellular signaling domain. In further embodiments, the intracellular signaling domain comprises one or more costimulatory signaling domains and an immunoreceptor tyrosine activation motif-containing signaling domain. In yet further embodiments, the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83. In some embodiments, the immunoreceptor tyrosine activation motif-containing signaling domain comprises a CD3 zeta signaling domain.

In some embodiments, the receptor or receptor subunit is a TCR or TCR subunit.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 illustrates the concept of an autonomous antigen-induced immune modulator and a constitutive immune receptor expression in a single vector. The immune receptor (CAR or TCR) is expressed constitutively. Ligation with its cognate ligand rewires endogenous NFAT signaling to the expression of an immune modulator molecule from the same lentiviral construct which is integrated as a single module.

FIG. 2 illustrates the design of a lentiviral transfer plasmid construct combining a constitutive portion and an inducible portion in a single lentiviral vector. The constitutive portion enables strong EF-1α promoter (or any other constitutive promoter that enables strong expression in immune cells)-driven expression of a reporter gene mCherry, which may be exchanged for a CAR or TCR of choice in an engineered immune cell. The inducible portion of the construct enables autonomous response to CAR/TCR engagement by a specific antigen or other tumor microenvironment characteristic signaling in engineered immune cells. This was achieved by rewiring endogenous CAR/TCR or other tumor microenvironment characteristic signaling to the expression of the effectors through activation of introduced promoters responsive to transcriptional factor that is increased when immune cells are specifically activated or localized to a given microenvironment, e.g., a tumor microenvironment, including a synthetic NFAT promoter, NF-κB promoter, a CD69 promoter, a CD137 promoter or a synthetic hypoxia-responsive element (HRE) promoter. The inducible portion drives expression of reporter gene eGFP, which may be exchanged by the effector of choice to increase safety and efficacy of immunotherapy.

FIG. 3 illustrates autonomous NFAT-driven expression of eGFP and constitutive expression of mCherry from a single lentiviral vector in a Jurkat cell line. Jurkat cells were transduced with different lentiviruses (pASP8, pASP9, pASP4.2, pASP7 and pASP5) at a multiplicity of infection (MOI) of 5 (MOI5). 48 h after transduction cells were stimulated with a cell stimulation cocktail (ionomycin and phorbol myristate acetate) and expression of reporter genes was monitored by confocal fluorescence microscopy and flow cytometry 76 h after transduction (24 h after stimulation).

FIGS. 4A-4C illustrate autonomous NFAT-driven expression of eGFP and constitutive expression of mCherry from a single lentiviral vector in primary human T cells. Primary donor-derived T cells were transduced with different lentiviruses. FIG. 4A illustrates that primary donor-derived T cells were transduced with pASP8 or pASP9. After transduction at a multiplicity of infection (MOI) of 5, the cells were expanded according to the standard protocol using anti-CD3/CD28 beads. At day 14 when cells were rested and hence NFAT-signaling was no longer active, the cells were again stimulated with CSC (ionomycin and phorbol myristate acetate), anti-CD3/CD28 beads or anti-CD3/CD28 antibody-coated plates and expression of reporter genes was monitored by confocal fluorescence microscopy and flow cytometry after 24 h. FIG. 4B illustrates that primary donor-derived T cells were transduced with pASP4.2 or pASP7. After transduction at a multiplicity of infection (MOI) of 5, the cells were expanded according to the standard protocol using anti-CD3/CD28 beads. At day 14 when cells were rested and hence NFAT-signaling was no longer active, the cells were again stimulated with CSC (ionomycin and phorbol myristate acetate), anti-CD3/CD28 beads or anti-CD3/CD28 antibody-coated plates and expression of reporter genes was monitored by confocal fluorescence microscopy and flow cytometry after 24 h. FIG. 4C illustrates that primary donor-derived T cells were transduced with pASP5. After transduction at a multiplicity of infection (MOI) of 5, the cells were expanded according to the standard protocol using anti-CD3/CD28 beads. At day 14 when cells were rested and hence NFAT-signaling was no longer active, the cells were again stimulated with CSC (ionomycin and phorbol myristate acetate), anti-CD3/CD28 beads or anti-CD3/CD28 antibody-coated plates and expression of reporter genes was monitored by confocal fluorescence microscopy and flow cytometry after 24 h.

FIG. 5 illustrates the capacity of the activated system. The capacity of the inducible activated pASP5 architecture is similar to that of a constitutively expressed system, whereas expression from the pASP4.2 architecture enables lower expression levels. Cells were activated with CSC (ionomycin and phorbol myristate acetate).

FIG. 6 illustrates the reversibility of the system. Transduced Jurkat cells were plated on 96-well plates and monitored in real-time for inducible eGFP and constitutive mCherry expression using the IncuCyte® system. Cells were stimulated with CSC (ionomycin and phorbol myristate acetate) for 24 h to monitor the increase of inducible eGFP expression. After 24 h cells were washed to remove the stimulus and left unstimulated for 120 h, when a drop in eGFP signal was observed. After 120 h they were re-stimulated to show an inducible eGFP response to the second round of stimulation.

FIGS. 7A-7B illustrate that designer T cells respond specifically to an antigen-positive adherent cancer cell line. FIG. 7A: Primary T cells were activated, transduced with pASP30 (inducible part placed in reverse in combination with a Her2-specific CAR comprising a 4D5 anti-Her2 scFv, a 4-1BB costimulatory domain, and CD3-zeta), expanded and rested according to the standard protocol and then co-cultured with the SKOV3 cancer cell line that expresses high levels of Her2 at various effector to target (E:T) ratios. As a negative control, an MDA468 cell line that does not express significant levels of Her2 was used. As a positive control, cells were non-specifically activated with CSC (ionomycin and phorbol myristate acetate). System performance was monitored using fluorescence microscopy and flow cytometry while killing capacity was determined by a standard luciferase based assay. FIG. 7B: Primary T cells were activated, transduced with pASP31 (inducible part placed in reverse in combination with 4D5 anti-Her2 scFv in combination with a CD28 costimulatory domain and CD3-zeta), expanded and rested according to the standard protocol and then co-cultured with the SKOV3 cancer cell line that expresses high levels of Her2 at various effector to target (E:T) ratios. As a negative control an MDA468 cell line that does not express significant levels of Her2 was used. As a positive control, cells were non-specifically activated with CSC (ionomycin and phorbol myristate acetate). System performance was monitored using fluorescence microscopy and flow cytometry while killing capacity was determined by a standard luciferase-based assay.

FIGS. 8A-8D illustrate that designer T cells respond specifically to an antigen-positive suspension cancer cell line. Primary human T cells were activated, transduced with pASP28 (NFAT-inducible eGFP in combination with constitutive anti-CD20 scFv+4-1BB+CD3-zeta CAR), expanded and rested according to the standard protocol. FIG. 8A: Anti-CD20 CAR expression and CSC (ionomycin and phorbol myristate acetate) inducible eGFP expression was determined by flow cytometry. FIG. 8B: Engineered T cells were co-cultured with CD20 positive or negative cell lines at E:T ratio 5:1 for 24 hours to show inducible and antigen-dependent expression of eGFP as determined by MFI. FIG. 8C: Antigen-dependent cell lysis that led to associated eGFP upregulation was demonstrated by flow cytometry where target and effector cells were differentiated as follows: GL-1 cell line was engineered to express GFP and samples were stained with anti-CD3, K562 cell lines were stained for characteristic Glycophorin A expression and NALM6 cell line was stained for CD19 expression. FIG. 8D: CD20 target antigen expression.

FIGS. 9A-9B illustrate that the all-in-one lentiviral system has superior functionalities compared to a two-component lentiviral system. FIG. 9A: Primary T cells were activated, transduced at MOI3 with pASP30 (NFAT-inducible eGFP in combination with constitutive 4D5 anti-Her2 scFv 4-1BB/CD3-zeta CAR in the same lentiviral construct) in a combination with pP7 (4D5 anti-Her2 scFv 4-1BB/CD3-zeta CAR same as used in pASP30 construct), expanded and rested according to the standard protocol. Cells were then non-specifically activated with CSC (ionomycin and phorbol myristate acetate) or co-cultured with SKOV3 cancer cell line that expresses high levels of Her2 at 3:1 effector to target (E:T) ratio. As a control, the MDA468 cell line that does not express significant levels of Her2 was used. System performance was monitored using fluorescence microscopy and flow cytometry. FIG. 9B: Primary T cells were activated, transduced at MOI3 with pASP5 (same construct as pASP30 but with CAR exchanged by mCherry) in a combination with pP7 (4D5 anti-Her2 scFv 4-1BB CAR same as used in pASP30 construct), expanded and rested according to the standard protocol. Cells were then non-specifically activated with CSC (cell stimulation cocktail; ionomycin and phorbol myristate acetate) or co-cultured with SKOV3 cancer cell line that expresses high levels of Her2 at 3:1 effector to target (E:T) ratio. As a control, the MDA468 cell line that does not express significant levels of Her2 was used. System performance was monitored using fluorescence microscopy and flow cytometry.

FIGS. 10A-10C illustrate on demand and reversible expression of human IL-12 (hIL-12) to increase efficacy of engineered primary T cells. FIG. 10A: Primary human T cells were activated, transduced with pASP18, expanded and rested according to the standard protocol and then subjected to an ON-OFF-ON activation regime to show expression of hIL-12 and reversibility of the system. Green arrows depict stimulation, black arrows depict washing by media exchange. FIG. 10B: pASP38 (NFAT-inducible hIL-12 in combination with constitutive 4D5 anti-Her2 scFv 4-1BB CAR)-transduced primary T cells lysed antigen-positive target cells at comparable levels as control pASP26-transduced cells, where hIL-12 was exchanged by eGFP (pASP26). FIG. 10C: Lysis was measured after 24 h co-culture and hIL-12 (left), IFN-γ (middle) and IL-2 (right) levels in supernatants from the 24 h co-culture were measured by ELISA.

FIGS. 11A-11C illustrate on demand tailored expression of an IL-6 receptor blocking antibody in primary engineered T cells. Primary human T cells were activated, transduced with pASP52 (NFAT-inducible anti-hIL6R scFV-Fc in combination with constitutive anti-CD20 scFv 4-1BB+CD3-zeta CAR), expanded and rested according to the standard protocol. FIG. 11A: Anti-CD20 CAR expression was determined by flow cytometry. FIG. 11B: Engineered T cells were co-cultured with CD20 positive or negative cell lines at E:T ratio 3:1 for 72 hours to show inducible and antigen-dependent expression and secretion of hIL-6R blocking antibody as determined by ELISA in the supernatant. FIG. 11C: Killing capacity in the same experiment was determined by flow cytometry. As a positive control, cells were non-specifically activated with CSC (ionomycin and phorbol myristate acetate). Target cell lines were differentiated from the effector T cells in the flow panel as follows: the GL-1 cell line was engineered to express GFP, K562 cell lines were stained for characteristic Glycophorin A expression and the NALM6 cell line was stained for CD19 expression.

FIGS. 12A-12D illustrate that the ATG sequence between the minimal promoter and the spacer does not influence systems performance. Jurkat cell line or primary T cells were engineered with two versions of a single-vector system that combines inducible eGFP+constitutive mCherry at MOI5. Vectors differ in the 6 nucleotide sequence between the minimal promoter and the spacer in the inducible module. CATATG 6-nucleotide sequence (SEQ ID NO: 1) and CCCGGG 6-nucleotide sequence (SEQ ID NO: 2) perform similarly in terms transduction efficacy, leakage and maximal activity of the system as determined by flow cytometry and fluorescence microscopy. TSS in the figure means Translational Start Site. FIG. 12A: Jurkat cells were transduced with two different lentiviruses described above (Vectors differ in the 6 nucleotide sequence between the minimal promoter and the spacer in the inducible module. Left: CATATG 6-nucleotide sequence (SEQ ID NO: 1) and Right: CCCGGG 6-nucleotide sequence (SEQ ID NO: 2) at a multiplicity of infection (MOI) of 5 (MOI5). 48 h after transduction cells were stimulated with a cell stimulation cocktail (ionomycin and phorbol myristate acetate) and expression of reporter genes was monitored by confocal fluorescence microscopy and flow cytometry 76 h after transduction (24 h after stimulation). FIG. 12B: Primary donor-derived T cells were transduced with two different lentiviruses described above (Vectors differ in the 6 nucleotide sequence between the minimal promoter and the spacer in the inducible module. Left: CATATG 6-nucleotide sequence (SEQ ID NO: 1) and Right: CCCGGG 6-nucleotide sequence (SEQ ID NO: 2)) at a multiplicity of infection (MOI) of 5 (MOI5). After transduction, the cells were expanded according to the standard protocol using anti-CD3/CD28 beads. At day 14 when cells were rested and hence NFAT-signaling was no longer active, the cells were again stimulated with CSC (ionomycin and phorbol myristate acetate) and expression of reporter genes was monitored by confocal fluorescence microscopy and flow cytometry after 24 h. FIG. 12C: Median fluorescence intensity of eGFP and CAR expression was measured in transduced population of Jurkat cells (described in 12A). FIG. 12D: Median fluorescence intensity of eGFP and CAR expression was measured in transduced population of primary human T cells (described in 12A).

FIGS. 13A-13B illustrate re-expression of the CAR after its initial target cell-mediated downregulation. Primary T cells were activated, transduced with a single-vector system pASP30 (inducible eGFP+constitutive 4D5 anti-Her2 scFv 4-1BB+CD3-zeta CAR) at MOI5, expanded and rested according to the standard protocol. At the end of the expansion they were sorted for CAR+cell population (Day 0). Cells were then co-cultured with SKOV3 cancer cell line that expresses high levels of Her2 at 0.3:1 effector to target ratio. Two days later cells were stained for CAR expression and profound downregulation of CAR was observed in cells that recognized target antigen as evidenced by upregulated NFAT inducible eGFP module in this population. Five days after the co-culture was set up, CAR T cells lysed a large majority of SKOV3 target cells which led to the re-expression of CAR. This demonstrated target mediated downregulation and re-expression of CAR on the cell surface.

FIGS. 14A-14B illustrate that the single lentiviral system has superior functionalities compared to a two-component lentiviral system. FIG. 14A shows primary T cells were activated, transduced with a combination of two-component pASP5 (inducible eGFP+constitutive mCherry) and pP7 (4D5 anti-Her2 scFv 4-1BB+CD3-zeta CAR) each at MOI3 or with single-vector system pASP30 (inducible eGFP+constitutive 4D5 anti-Her2 scFv 4-1BB+CD3-zeta CAR) alone at MOI3, expanded and rested according to the standard protocol. Cells were then non-specifically activated with ionomycin and phorbol myristate acetate. System performance was monitored using fluorescence microscopy and flow cytometry. As expected, in the two-component system (left) there are 3 populations of transduced CAR T cells (each at approximately 30% frequency in regards to transduced population): constitutive CAR module positive only, inducible eGFP module positive only (activated because of nonspecific stimulation with PMA and ionomycin) and double positive. Only double positive population is functional and would need to be sorted out before therapeutic implementations. In contrast, the single-vector system (right) confers homogenous population of double positive constitutive CAR and inducible eGFP modules in primary T cells, because both modules are integrated together as a single molecule into the same genomic locus. FIG. 14B shows primary T cells were generated as in FIG. 14A but activated specifically through co-culture with SKOV3 cancer cell line that expresses high levels of Her2 at 3:1 effector to target (E:T) ratio. The MDA468 cell line that does not express significant levels of Her2 was used as a control. As a consequence of single-construct engineering, in contrast to two-component system, single-vector system provided increased expression of the inducible module in CAR positive population (MFI in eGFP channel, left panel), overall higher eGFP positive frequency of CAR T cells (middle panel) and increased ratio of eGFP positive vs eGFP negative cells in the CAR positive population. Presence of CAR negative and eGFP positive population in the CAR T cells engineered with single-vector system (pASP30) and activated with SKOV3 cells is due to the downregulation of CAR after recognizing target antigen. This phenomena was investigated and demonstrated in FIGS. 13A-13B (Re-expression of the CAR after its initial target cell-mediated downregulation) in this document.

FIGS. 15A-15E illustrate in vivo validation of the single-vector system. Primary T cells were activated, transduced with pASP26 (inducible eGFP+constitutive 4D5 anti-Her2 scFv 4-1BB+CD3-zeta CAR; forward orientation) at MOI5, expanded and rested according to the standard protocol. Engineered primary T cells were injected intravenously (i.v.) at 3 different amounts (0.15/0.3/1 million of CAR+cells per mouse) into NSG mice bearing subcutaneous (s.c.) xenograft SKOV3 tumors. FIG. 15A shows that 1 million of SKOV3 cells (engineered to express luciferase) were injected s.c. 4 days prior to the administration of CAR T cells to establish tumors. 1 million of CAR+primary T cells completely controlled tumor growth as evidenced by no detectible tumor measured by imaging. FIG. 15B shows quantification of luciferase activity (upper panel: average of groups and lower panel: individual mice on day 25), FIG. 15C shows caliper measurement of tumor growth (upper panel: average of groups and lower panel: individual mice on day 28). FIG. 15D shows survival. FIG. 15E shows blood counts of human primary T cells in mice correlated with tumor control where only in highest dose of CAR+ T cells persistence was observed.

FIG. 16 illustrates that engineered anti-hIL-6R scFv-Fc is secreted from activated engineered primary T cells and binds cell surface hIL-6Ra. Primary T cells were activated, transduced with pASP52 (inducible anti-hIL-6R-Myc tag+constitutive anti-CD20 scFv 4-1BB+CD3-zeta CAR; back-to-back orientation) or control pP2 construct (constitutive anti-CD20 scFv 4-1BB+CD3-zeta CAR) at MOI5, expanded and rested. Engineered T cells were co-cultured with CD20 positive cell line NALM6 at 3:1 ratio for 72 h to induce antigen-dependent expression and secretion of hIL-6R blocking antibody. Supernatant from these co-cultures was added to the HEK 293T cells engineered to express hIL-6Ra or control parental cells (with no detectable IL-6Ra expression) and incubated for 1 h. Binding anti-hIL-6R scFv-Fc-Myc tag was detected by secondary stain against Myc tag. Only supernatant from anti-hIL-6R scFv-Fc producing, but not control engineered T cells stained target IL-6Ra expressing cells in a dose dependent manner. The same supernatant did not stain parental HEK 293 T cells demonstrating specific binding. Commercial recombinant anti-hIL-6R antibody was used as a positive control. SN means supernatant.

FIGS. 17A-17B are a series of graphs. FIG. 17A illustrates that secreted anti-hIL-6R scFv-Fc blocks hIL-6Ra surface staining by recombinant anti-hIL-6Ra Ab. FIG. 17B shows quantification of decreased hIL-6Ra surface staining by anti-IL-6Ra antibody. Designed anti-hIL-6R scFv-Fc was constitutively expressed in HEK 293T cells and secreted in the media. Supernatant from these cells was added to the HEK 293T cells engineered to express hIL-6Ra and incubated 16 h. Cells were then stained with recombinant commercial anti-hIL-6Ra antibody and binding of this antibody was monitored by flow cytometry (histogram in upper panel). MFI of hIL-6Ra staining was measured (lower graph). Only supernatant from anti-hIL-6R scFv-Fc producing, but not control cells blocked staining of target IL-6Ra expressing cells in a dose dependent manner. Clinically used Tocilizumab was used as a positive control. SN means supernatant.

FIG. 18 illustrates that secreted anti-hIL-6R scFv-Fc inhibits hIL-6 signaling. To further demonstrate biological activity of secreted engineered human IL-6Ra-blocking antibody, we investigated whether its binding to human IL-6Ra expressed on the cell surface would inhibit IL-6 signaling. Only supernatant from anti-IL6R scFv-Fc producing HEK 293T cells, but not control cells secreting irrelevant antibody, inhibited IL-6 signaling monitored through activity of STAT3 reporter in HEK 293T cells engineered with pASP59 (STAT3-eGFP+hIL-6Ra).

FIGS. 19A-19C illustrate that antigen-inducible transcriptional factors TCF-7 and FOXO1-3A mediate less differentiated status and improved antigen-dependent proliferation in CAR T cells. Primary T cells were activated, transduced with pASP30 (inducible eGFP+constitutive 4D5 anti-Her2 scFv 4-1BB+CD3-zeta CAR; back-to-back orientation), pASP72 (inducible hTCF-7+constitutive 4D5 anti-Her2 scFv 4-1BB+CD3-zeta CAR; back-to-back orientation) or pASP73 (inducible human FOXO1-3A+constitutive 4D5 anti-Her2 scFv 4-1BB+CD3-zeta CAR; back-to-back orientation) at MOI5, expanded and rested according to the standard protocol. FIG. 19A shows that engineered cells were subjected to 2 repeated rounds of target cells exposure and their phenotype was determined 3 days after last stimulation using multicolor flow cytometry. FIG. 19B shows that engineered cells were sorted for CAR positive populations, subjected to 2 repeated rounds of target cells exposure, rested 5 days, stained with CellTrace Violet and re-exposed to Her2 positive target cells for 5 days. Proliferation was measured by flow cytometry. FIG. 19C shows that engineered cells were subjected to 3 repeated rounds of target cells exposure and 6 days after last exposure, Lag3 expression was determined by flow cytometry.

FIG. 20 illustrates that CAR T cells engineered with antigen inducible expression of transcriptional factors TCF-7 and FOXO1-3A retain capacity to lyse tumor cells. Primary T cells were activated, transduced with pASP30 (inducible eGFP+constitutive 4D5 anti-Her2 scFv 4-1BB+CD3-zeta CAR; back-to-back orientation), pASP72 (inducible hTCF-7+constitutive 4D5 anti-Her2 scFv 4-1BB+CD3-zeta CAR; back-to-back orientation) or pASP73 (inducible human FOXO1-3A+constitutive 4D5 anti-Her2 scFv 4-1BB+CD3-zeta CAR; back-to-back orientation) at MOI5, expanded and rested according to the standard protocol. Lysis capacity of Her2 positive cell line SKOV3 was determined after 2 and 3 rounds of antigen exposure. The Her2 negative MDA468 cell line was used as a control.

FIG. 21 illustrates adaptation of the single-vector system to sense and respond to IL-6. CAR was exchanged with IL-6Ra and NFAT-sensing promoter was exchanged with STAT3-sensing promoter in the single-vector system to generate pASP59 (STAT3-eGFP+hIL-6Ra). HEK 293 T cells were transduced with pASP59 and stimulated with increasing amounts of human IL-6. Specific and dose dependent responses were observed, demonstrating that modular design of the single-vector system where elements (receptors and promoters) can be easily exchanged to detect customized signals and respond by a relevant therapeutic output.

FIGS. 22A-22B illustrate use of the single-vector system to screen for antigen specific immune cells. FIG. 22A shows patient derived Tumor-Infiltrating Lymphocytes (TILs) were successfully transduced with pASP5 (inducible eGFP+constitutive mCherry). Inducible module was upregulated when engineered TILs were stimulated with ionomycin and phorbol myristate acetate. Co-culture of engineered TILs with matching autologous tumor sample led to increased signature of inducible eGFP. FIG. 22B shows engineered TILs, co-cultured with autologous tumor cells, were stained with standard activation markers used in determining antigen-specific immune cells. Correlation between these markers and NFAT-inducible eGFP was investigated.

FIGS. 23A-23C illustrate IL13Rα2-targeting CAR-inducible secretion of functional bispecific T cell engager pASP79 (BiTE). Primary T cells engineered with single-vector IL13Rα2-targeting CAR and inducible BiTE (pASP79; the BiTE targeting EGFR wild type (EGFRwt) and EGFR variant III (EGFRvIII) proteins) were cultured in RPMI1640 medium, IL13Rα2- or IL13Rα2+glioma stem cell lines for two days. Supernatant was collected and used in ELISA-based detection of BiTE and in a bioluminescence killing assay. FIGS. 23A-23B are graphs showing ELISA results. Plates were coated with EGFRwt or EGFRvIII proteins, which are the targets of BiTEs in this study. Direct ELISA was used to detect the induced secretion of BiTEs. FIG. 23C is a graph showing ELISA results. Un-transduced T cells were co-cultured with an EGFRvIII+glioma stem cell line in the collected supernatant. The ratio of T cells to tumor cells was is 5:1. 12 hours later, the bioluminescent signal was determined. One way ANOVA was used to detect the statistical differences between each group. *P<0.05, **P<0.01,****P<0.0001.

FIGS. 24A-24C illustrate IL13Rα2-targeting CAR-inducible secretion of functional bispecific T cell engager pASP83 (BiTE). Primary T cells engineered with single-vector IL13Rα2-targeting CAR and inducible BiTE (pASP83; the BiTE targeting EGFR wild type (EGFRwt) and EGFR variant III (EGFRvIII) proteins) were cultured in RPMI1640 medium or IL13Rα2+glioma stem cell lines for two days. Supernatant was collected and used in ELISA based detection of BiTE and in a bioluminescence killing assay. FIGS. 24A-24B are graphs showing ELISA results. Plates were coated with EGFRwt or EGFRvIII proteins, which are the targets of BiTEs in this study. Direct ELISA was used to detect the inducible secretion of BiTEs. FIG. 24C is a graph showing ELISA results. Un-transduced T cells were co-cultured with an EGFRvIII+glioma stem cell line in the collected supernatant. The ratio of T cells to tumor cells was is 5:1. 12 hours later, the bioluminescent signal was determined. One way ANOVA was used to detect the statistical differences between each group. Thus, pASP83 CAR T cells secrete biologically active EGFR-specific BiTEs in response to antigen activation, and the secreted BiTEs can redirect the activity of untransduced T cells against EGFR-expressing cancer cells. **P<0.01,****P<0.0001.

FIGS. 25A-25B illustrate the synNotch single-vector system. FIG. 25A is a schematic of the synNotch single-vector system. The constitutive EF1alpha promoter leads to the continuous expression of a HER2-specific synNotch receptor (4D5-Notch1-Gal4-VP64) and a blue fluorescent BFP2 reporter protein. Binding of the synNotch receptor to its target antigen HER2 induces Notch1 cleavage which releases the transcription factor Gal4-VP64 from the receptor. Gal4-VP64 binds to the Gal4 enhancer sequence (Gal4-UAS) thereby activating the minimal CMV promoter (mCMV) and leading to the production of a red fluorescent mCherry reporter protein. “AAA” represents a synthetic poly-A sequence and hCD8alpha indicates a secretion sequence. P2A is facilitating the co-expression of receptor and BFP2 reporter proteins. FIG. 25B shows experimental validation of the synNotch single-vector system. Representative flow-cytometry plots show expression of BFP2 and mCherry in primary human T cells transduced with the synNotch single-vector construct. Transduced T cells were cultured in the absence (unstimulated) or presence (stimulated) of HER2-expressing SKOV3 tumor cells for 24 h and analyzed with a LSRFortessa flow-cytometer (BD Biosciences).

FIG. 26 illustrates pASP73 (inducible human FOXOI-3A+constitutive 4D5 anti-Her2 scFv 4-1BB+CD3-zeta CAR; back-to-back orientation).

FIG. 27 illustrates constructs that express a CAR and a transcription factor under control of a single constitutive promoter. The constructs include a 2A peptide spacer between the CAR and the transcription factor.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa (κ) and lambda (λ) light chains refer to the two major antibody light chain isotypes.

The term “synthetic antibody” as used herein, refers to an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized or derived from a biological sample. Such a biological sample can include, but is not limited to a tissue sample, a tumor sample, a cell or a biological fluid.

The term “anti-tumor effect” as used herein, refers to a biological effect observed when treating solid tumors which manifests in a variety of ways, including, for example, by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” refers to any material derived from the same individual to which it is later re-introduced into the individual.

“Allogeneic” refers to a graft or infusion derived from a different animal of the same species.

“Xenogeneic” refers to a graft or infusion derived from an animal of a different species.

The term “cancer” as used herein refers to a disease characterized by the rapid and uncontrolled growth of aberrant cells. Cancer cells can grow locally in its tissue of origin or spread through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “chimeric antigen receptor” or “CAR,” as used herein, refers to a non-MHC-restricted synthetic antigen-specific immune receptor that is engineered to be expressed on an immune effector cell. CARS are commonly used to engineer T cells, subsequently referred to as “CAR T cells”. To make a CAR T cell, T cells are removed from a patient and modified to express one or more receptors specific to a particular antigen(s) of interest. CARs are delivered to patients generally as an intravenous infusion through a process referred to as adoptive cell transfer. In some embodiments, the CARs specifically bind a tumor-associated antigen or a tumor-specific antigen, for example. CARs may also comprise an intracellular activation domain, a transmembrane domain and an extracellular domain comprising a tumor-associated antigen binding domain or a tumor-specific antigen binding domain. In some aspects, CARs constructs comprise nucleic acids encoding (I) a single-chain variable fragment (scFv) targeting a clinically-relevant antigen derived from a monoclonal antibody, (2) a transmembrane domain, (3) one or more costimulatory domains, and (4) an ITAM-containing signaling domain such as CD3-zeta. In some embodiments, a CAR can target cancers by redirecting the specificity of a T cell expressing the CAR specific for a tumor-associated antigen or a tumor-specific antigen.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MEW molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type II: CRISPR/Cas system, short segments of foreign DNA, termed “spacers” are integrated within the CRISPR genomic loci are transcribed and processed into short CRISPR RNA (crRNA). These cr:RNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/Cas mediated genome editing and regulation, highlighted its transformative potential for basic science, cellular engineering and therapeutics.

The term “CRISPRi” refers to a CRISPR system for sequence specific gene repression or inhibition of gene expression at the transcriptional level.

“Effective amount” or “therapeutically effective amount” are used interchangeably herein, each referring to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “effector” as used herein refers to a molecule or a protein or fragment thereof that alters, moderates, interferes with or blocks a signaling axis or a transcription modulator or fragment thereof that provides benefit to immune cell function. In some embodiments, effectors comprise effectors for: (1) limiting toxicities (sensing/preventing CRS, sensing/preventing cerebral toxicities); (2) overcoming checkpoint inhibition; (3) recruiting endogenous immune responses and improving persistence; (4) improving penetrance; and/or (5) improving persistence; (6) shifting engineered T cells into a preferred phenotype that provides therapeutic benefit; (7) modulating gene expression that provides benefit to immune cell function. In some embodiments, the effector blocks the IL-6 or another inflammatory signaling axis. The effector may be a cytokine, an interleukin, an interferon, a chemokine, a receptor, a ligand, an antibody or antibody fragment, a bispecific antibody, a checkpoint inhibitor antagonist, an agonist, an enzyme, a regulatory element, a transcription factor, or a DNA binding domain of a transcription factor.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture.

The term “ex vivo” as used herein refers to cells that have been removed from a living organism, (e.g., a human) and propagated outside the organism (e.g., in a culture dish, test tube, or bioreactor).

The term “expression” as used herein refers to the transcription and/or translation of a particular nucleotide sequence driven by an endogenous or exogenous promoter.

“Expression vector” refers to a vector comprising a recombinant polynucleotide comprising expression control sequences. The expression control sequences may be operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “receptor fusion protein” as used herein refers to a fusion protein that comprises an antigen-binding domain. In some embodiments, the receptor fusion protein comprises an antigen-specific synNotch receptor. In some embodiments, the receptor fusion protein comprises a CAR.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

The term “sequence identity” as used herein refers to the degree of sequence identity between two polymeric molecules particularly between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “lentivirus” as used herein refers to viruses in a genus of the Retroviridae family. Lentiviruses are unique among the retroviruses in being able to infect non-dividing cells. In addition, they can deliver a significant amount of genetic information into the DNA of the host cell, so lentivirus-derived vectors are one of the most efficient gene delivery vectors available. HIV, SIV, and FIV are all examples of lentiviruses.

The term “modified” as used herein, refers to a changed state or structure of a molecule or cell of the invention. Molecules may be modified in many ways, including chemically, structurally, and functionally. Cells may be modified through the introduction of nucleic acids.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” or “overexpression” in reference to a tumor antigen indicates an increased level of expression compared to the level of expression in a normal cell from that tissue or organ. Patients having solid tumors or a hematological malignancy characterized by overexpression of a tumor antigen can be identified by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “nucleic acid” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably to refer to polymers of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short amino acid polymers, which also commonly are referred to in the art as peptides, oligopeptides and oligomers, for example, and to longer amino acid polymers, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein refers to a DNA sequence recognized by the synthetic machinery of the cell, or introduced synthetic machinery, required to initiate the transcription of a specific polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

The term “minimal promoter (P_{MIN})” as used herein refers to a TATA-box promoter element upstream of the inducible gene and sequence can be located between the −35 to +35 region with respect to transcription start site (Smale ST (2001) Core promoters: active contributors to combinatorial gene regulation. Genes Dev 15: 2503-2508). Eukaryotic promoters of protein-coding genes have one or more of three conserved sequences in this region (i.e. the TATA-box, initiator region, and downstream promoter element). A minimal promoter enables low basal leakiness in the absence of specific transcriptional activators and high expression when transcription activators are bound upstream of minimal promoter at their specific DNA binding sites. In some embodiments the minimal promoter is derived from the pGL4.23[luc2/minP]; Promega and validated in (Smole, A., Lains̆c̆ek, D., Bezeljak, U., Horvat, S. & Jerala, R. A Synthetic Mammalian Therapeutic Gene Circuit for Sensing and Suppressing Inflammation. Mol. Ther. 25, 102-119 (2017)). Alternative minimal promoters can be used, such as minimal TATA box promoter, minimal CMV promoter or minimal IL-2 promoter.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only in the presence or absence of certain conditions such as, for example, when an inducer (e.g., metal ions, alcohol, oxygen, etc.) which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell, or across a cell membrane. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

The term “subject” is intended to include living organisms in which an immune response can be elicited (e.g., mammals). A “subject” or “patient,” as used therein, may be a human or non-human mammal. Non-human mammals include, for example, livestock and pets, such as sheep, cattle, pigs, cats, dogs, mice, and rats. Preferably, the subject is human.

As used herein, the term “T cell receptor” or “TCR” refers to a heterodimeric membrane protein that participates in the activation of T cells in response to the presentation of antigen. The TCR is responsible for recognizing antigens bound to major histocompatibility complex molecules. The TCR cannot transduce signals through the cell membrane, instead relying on the activity of CD3, another heterodimeric protein complexed with the TCR to form a TCR/CD3 complex. TCR is composed of a heterodimer of an alpha (α) and beta (β) chain, although in some cells the TCR consists of gamma (γ) and delta (δ) chains, TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR., including, for example, a helper T cell, a cytotoxic T cell, a memory T cell, regulatory T cell, natural killer T cell, and gamma delta T cell.

The term “therapeutic” as used herein describes a type of treatment and/or prophylaxis. A therapeutic effect is obtained by suppression, remission, or eradication of a disease state.

The terms “transfected” or “transformed” or “transduced” are used interchangeably herein to refer to a process by which exogenous nucleic acid is transferred or introduced into the host cell. A “transfected” or “transformed” or “transduced” cell is one which has been transfected, transformed or transduced with exogenous nucleic acid (e.g., a lentiviral vector comprising a CAR, a plasmid, etc.). The cell includes the primary subject cell actually transfected, transformed, or transduced and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, provided herein are methods for engineering immune cells by introducing genetic circuits that endow them with additional functionalities such as autonomous antigen-induced expression of immunomodulatory molecules limited to the tumor microenvironment. Provided herein is a broadly applicable expression platform that combines a constitutive immune receptor or receptor subunit, receptor fusion protein or fluorescent marker and an inducible effector in a clinically relevant setting. This platform provides a potentially field-changing approach to managing severe side effects associated with adoptive cell therapy, including cytokine release syndrome and neurological toxicities, by autonomously regulated and in-situ expression of an effector that blocks the IL-6 or another inflammatory signaling axis. To extend adoptive cell therapy from hematologic malignancies into the treatment of solid tumors, various immune enhancing molecules were integrated into the developed system in combination with clinically validated immune receptors.

Currently no vector system exists in the field of immunotherapy that enables simultaneous expression of constitutive and inducible modules, in part due to complexities of engineering lentiviral-derived constructs, where a fine balance between desired functionalities, low basal leakiness, maintaining high levels of maximal production and prevention of immunogenicity is required, while maintaining the titer of the virus high enough to enable transduction of primary immune cells. Limitations of two-vector systems include non-homogenous cell populations in which only a part of the population receives both components. Consequently, laborious and often therapeutically non-feasible selection procedures are needed.

A disadvantage of having inducible and constitutive modules on two separate vectors is that it may result in non-homogeneous modification of engineered cells in which only a part of the population receives both components, resulting in a large part of modified cells being non-functional. Furthermore, even in double transduced cells, system behavior is unpredictable since different amounts of the inducible and constitutive modules may be integrated into the different genomic loci in the genome. Consequently, laborious and often therapeutically non-feasible selection procedures are often needed to obtain something that works as expected with a two-vector system. Thus, the all-in-one system of the invention has several advantages in that it enables the manufacture of a well-defined cell product with a more homogeneous transgene distribution.

Another advantage of the all-in-one system is its simplified modular assembly, where only one virus need be produced to achieve constitutive expression of a transgene, for example a CAR or TCR or TCR subunit, and inducible expression of an effector.

An important aspect for the use of intracellular effectors to modify functionalities of T cells, such as transcriptional factors or regulatory element or a DNA binding domain of a transcription factor, for example, T-bet, TCF7, EOMES, a Runx family member, BLIMP1, Bc12, Bc16, FoxP3, FoxO1, FoxO1-3A, or a portion thereof, or any other transcription factor providing benefit to immune cell function when overexpressed, is that in order to turn on expression of these effectors and modify functionality of the engineered cell, an inducible part is needed that carries genetic information for expression of such regulatory element in the same cell as constitutively expressed immune receptor, that drives endogenous signaling to activate inducible part. So, if the constitutive and inducible parts were on separate plasmids, a large majority of the transduced population would be non-functional. This is of course also true for secreted effectors, however since they are secreted, they can still have an effect, because they influence all the cells in the vicinity.

A further advantage of the all-in-one system is that it can be readily used, along with the functionalities that it has, as a donor template delivered with, for example, an adeno associated virus such as AAV6, for use with a CRISPR system, for example a CRISPR/Cas9 system, for homologous recombination-mediated knock-in into a desired locus, as described below. In some embodiments, the locus is the endogenous TCR of the cell, or an HLA or immune checkpoint or other molecule that negatively affects the immune cell function of interest of the engineered cell. This application would not be possible with a two-component system.

The present disclosure provides a broadly applicable all-in-one lentiviral system with highly optimized transcriptional elements. This will improve the safety of adoptive cell therapy by treating CRS and neurological toxicities in-situ by therapeutic cells themselves. Alternatively, the modular system enables the engineering of T cells to augment efficacy of treating solid tumors using CAR or TCR modified immune cells in adoptive cancer immunotherapy.

Provided is a viral vector comprising: a first polynucleotide comprising a constitutive promoter operably linked to a nucleic acid encoding at least one transgene, wherein one of the at least one transgenes encodes a receptor or receptor subunit, a receptor fusion protein, of a fluorescent marker; and a second polynucleotide comprising an inducible promoter operably linked to a nucleic acid encoding an effector.

In some embodiments, the at least one transgene encodes a receptor fusion protein. In some embodiments, the receptor fusion protein comprises an antigen-specific synNotch receptor and an optional reporter. In some embodiments, the receptor fusion protein further comprises a 2A peptide between the antigen-specific synNotch receptor and the reporter. In some embodiments, the reporter is BFP2. In further embodiments, the antigen-specific synNotch receptor fusion protein is a HER2-specific synNotch receptor fused to Gal4-VP64. In further embodiments, binding of the synNotch receptor to its target antigen HER2 induces Notch1 cleavage which releases the transcription factor Gal4-VP64 from the fusion protein.

In some embodiments, the transgene encodes a receptor or receptor subunit. In some embodiments, the receptor or receptor subunit is a TCR or TCR subunit. In further embodiments, the receptor is a TCR, wherein the TCR comprises an extracellular antigen binding domain capable of specifically binding a cognate antigen and an intracellular domain. In yet further embodiments, the intracellular domain drives expression of the effector when the antigen-binding domain of the TCR binds its cognate antigen by activating the inducible promoter operably linked to the effector.

In some embodiments, the receptor or receptor subunit is a cytokine receptor or cytokine receptor subunit. In some embodiments, the cytokine receptor subunit is IL-6Ra.

In some embodiments, the receptor fusion protein is a chimeric antigen receptor (CAR) comprising an extracellular antigen-binding domain capable of specifically binding a cognate antigen and an intracellular signaling domain, wherein the inducible promoter is capable of driving expression of the effector when the antigen binding domain binds its target.

In some embodiments, the receptor fusion protein is a CAR comprising an extracellular antigen-binding domain capable of specifically binding a cognate antigen and an intracellular signaling domain. In further embodiments, the intracellular signaling domain drives expression of the effector when the antigen-binding domain of the CAR binds its cognate antigen by activating the inducible promoter operably linked to the effector. In yet further embodiments, the inducible promoter is capable of driving expression of the effector when the receptor binds its ligand.

In some embodiments, the transgene encodes a fluorescent marker. In further embodiments, the fluorescent marker is mCherry.

In some embodiments, the first polynucleotide, the second polynucleotide or both the first and the second polynucleotides comprise an insulator sequence or a linker sequence.

In some embodiments, the second polynucleotide encodes more than one effector, wherein each effector is under the control of at least one promoter. In further embodiments, the at least one promoter is at least one inducible promoter.

In some embodiments, the viral vector is a retroviral vector, an adenoviral vector, or an adeno-associated viral vector. In further embodiments, the retroviral vector is a lentiviral vector.

In some embodiments, the inducible promoter and the constitutive promoter drive expression in the same direction, or in different directions.

In some embodiments, the CAR comprises an antigen binding domain, a transmembrane domain, and an intracellular signaling domain. In further embodiments, the intracellular signaling domain comprises one or more costimulatory signaling domains and an immunoreceptor tyrosine activation motif-containing signaling domain. In some embodiments, the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83. In some embodiments, the immunoreceptor tyrosine activation motif-containing signaling domain comprises a CD3 zeta signaling domain.

Also provided is an engineered cell comprising the viral vector disclosed herein. In some embodiments, the engineered cell is an immune cell or precursor cell thereof. In some embodiments, an endogenous TCR or TCR subunit or HLA (Human leukocyte antigen) or immune checkpoint molecule (such as PD-1, TIM3, LAG3, CTLA4, 2B4, CD160, CD5) of the engineered cell has been knocked out using a CRISPR-related system, for example a CRISPR/Cas9 system, with non-homologous end joining or homologous recombination where the viral vector serves as a donor template molecule. In some embodiments, at least one TCR subunit is knocked out using a CRISPR-related system, a TALEN or a Zinc finger nuclease. In some embodiments, the CRISPR-related system is CRISPR/Cas9. In further embodiments, both subunits of the endogenous TCR are knocked out.

Also provided is a method for generating an engineered cell, comprising (1) obtaining a sample comprising immune cells or precursors of immune cells; (2) stimulating/activating the cells; (3) introducing the viral vector; (4) expanding the cells and (5) resting the cells. In some embodiments, the engineered cell is an immune cell or precursor cell thereof. In some embodiments, the method further comprises the step of knocking out the endogenous TCR or HLA (Human leukocyte antigen) or immune checkpoint molecule (such as PD-1, TIM3, LAG3, CTLA4, 2B4, CD160, CD5) or other molecule that negatively affects immune cell function of interest of the engineered cell has been knocked out using a CRISPR-related system, for example a CRISPR/Cas9 system with non-homologous end joining or homologous recombination where the viral vector serves as a donor template molecule.

Provided is a method for treating a patient having a disease, disorder or condition associated with expression of an antigen, the method comprising administering to the patient an effective amount of a composition comprising the engineered cell of any one of the previous embodiments. In some embodiments, the expression of the antigen is abnormal. In some embodiment, the engineered cell is an immune cell or precursor cell thereof. In further embodiments, the engineered cell is an NK cell, a B cell, a dendritic cell or a macrophage. In some embodiments, the composition further comprises a pharmaceutically acceptable carrier or adjuvant.

Provided is an engineered immune cell or engineered immune cell precursor cell, comprising: an insertion in a gene locus, wherein the insertion comprises a first polynucleotide comprising a constitutive promoter operably linked to a nucleic acid encoding at least one transgene, wherein one of the at least one transgenes encodes a receptor or receptor subunit, a receptor fusion protein or a fluorescent marker; and a second polynucleotide comprising an inducible promoter operably linked to a nucleic acid encoding an effector. In some embodiments, the transgene encodes a receptor fusion protein. In some embodiments, the receptor fusion protein comprises an antigen-specific synNotch receptor and an optional reporter. In some embodiments, the receptor fusion protein further comprises a 2A peptide between the antigen-specific synNotch receptor and the reporter.

Promoters

In some embodiments, the constitutive promoter that is operably linked to the transgene is an EF-1alpha promoter or any other constitutive promoter that drives constitutive expression in immune cells (such as PGK-1, UBC, CMV, CAGG, SV40 or pan-hematopoietic promoter, such as vav). In some embodiments, the promoter is a human promoter.

In some embodiments, the inducible promoter that is operably linked to the polynucleotide encoding the effector is any promoter the activation of which is responsive to a transcriptional factor that is increased when immune cells are specifically activated or localized to a given microenvironment (could be tumor microenvironment), such as an NFAT promoter, a STAT3-sensing promoter, a CD69 promoter, a CD137 promoter, or a hypoxia-responsive element promoter. In some embodiments, the inducible promoter further comprises a minimal promoter operably linked to an inducible enhancer. In some embodiments, the promoter is a human promoter. NFAT-inducible promoter can be exchanged with any inducible promoter that specifically binds specific transcriptional factors. Hypoxia responsive element, for example, instead of NFAT responsive element, would bind HIF transcriptional factors and activate the system in hypoxic conditions. Without wishing to be bound by theory, if NFAT responsive element is present, it needs a signal from TCR/CAR or other immune receptor that induces NFAT signaling.

In further embodiments, the inducible promoter is linked to a minimal promoter (P_{MIN}). In some embodiments the minimal promoter is derived from the pGL4.23[luc2/minP]; Promega and validated in (Smole, A., Lains̆c̆ek, D., Bezeljak, U., Horvat, S. & Jerala, R. A Synthetic Mammalian Therapeutic Gene Circuit for Sensing and Suppressing Inflammation. Mol. Ther. 25, 102-119 (2017)). Alternative minimal promoters can be used, such as minimal TATA box promoter, minimal CMV promoter or minimal IL-2 promoter. In some embodiments, the minimal promoter is optimized for a desired level or rate of transcription. In some embodiments, an inducible promoter linked to a minimal promoter enables a medium ON state while remaining silent in the absence of a trigger signal. This configuration is of high value when no leakiness is acceptable due to potential toxicities, for example when using immune modulators such as cytokines. In some embodiments, an inducible promoter is linked to a minimal promoter that enables a high ON state. In some embodiments the system's properties are tailored and are optimized to the specific therapeutic needs. For the inducible expression of an immune augmenting molecule, a system with minimal leakage is required to prevent toxicities. Without wishing to be bound by theory, the level of maximal production might not be that important in the case of an immune augmenting molecule, or might even be deleterious if too high, given the potent immune-enhancing effect. For inducible expression of blocking antibodies, the system is optimized for high maximal expression (ON state).

Effector

The effector may be a cytokine, an interleukin, an interferon, a chemokine, a receptor, a ligand, an antibody or antibody fragment, a bispecific antibody, a BiTE, a checkpoint inhibitor antagonist, an agonist, an enzyme, a regulatory element, a transcription factor, or a DNA binding domain of a transcription factor. Transcriptional factors usually comprise DNA binding domain and the domain that modifies transcription (activates or represses). Part of transcriptional factor means that the DNA binding domain of the specific transcriptional factor may be used, which would act as a competitive inhibitor of endogenous transcriptional factor—for example, to suppress activity of transcriptional factor that leads to the exhaustion of engineered cells. On the other hand, the portion of NFAT that has transcriptional activation activity and the domain that enables entry into the nucleus only upon Ca-mediated signaling may be fused (meaning the DNA binding domains that enables binding to NFAT DNA binding site is removed) and then this part is fused with a different DNA binding domain (which could be also designable DNA binding domain such as ZFN, TALEN or Cas9) to rewire endogenous NFAT signaling to activation or repression (if we add repression domain such as KRAB) of any gene.

In some embodiments, the cytokine is an interleukin. In some embodiments, the interleukin is IL-2, IL-7, IL-12, IL-15, IL-18, or IL-21.

In some embodiments, the interferon is IFNα or IFNβ.

In some embodiments, the antibody or antibody fragment or bispecific antibody is anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-IL-8, anti-MCP1, anti-MIP-1, anti-TGFβ, anti-CD47, anti-CSF1R, anti-CD28, anti-TIGIT, anti-VEGFR, anti-FAP or any antibody that blocks cytokines, depletes or alters a particular cell subset, or alters immunity, survival, trafficking or signaling or increases efficacy of immune cells.

In some embodiments, the checkpoint inhibitor antagonist is anti-PD-L1, anti-PD-1, anti-CTLA4, anti-LAG3, anti-TIM3, anti-2B4, or anti-CD160, anti-CD5.

In some embodiments, the bispecific antibody comprises functional domains of any of but not limited to anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-IL-8, anti-MCP1, anti-MIP-1, anti-TGFβ, anti-CD47, anti-CSF1R, anti-CD28, anti-TIGIT, anti-VEGFR, anti-FAP or any antibody that blocks cytokines, depletes or alters a particular cell subset, or alters immunity, survival, trafficking or signaling or increases efficacy of immune cells or anti-PD-L1, anti-PD-1, anti-CTLA4, anti-LAG3, anti-TIM3, anti-2B4, or anti-CD160, anti-CD5 or any other immune checkpoint molecule.

In some embodiments, the chemokine is CCL5 (RANTES), XCL-1, XCL-2, CCR-7, CCL-19, CCL-21.

In some embodiments, a transcription factor or regulatory element or a part of transcription factor (derived from the transcription factor) is T-bet, TCF7, EOMES, a Runx family member, BLIMP1, Bc12, Bc16, FoxP3, FoxO1, FoxO1-3A, or a portion thereof, or any other transcription factor providing benefit to immune cell function when overexpressed. In some embodiments, the enzyme is heparinase, collagenase, or a metalloproteinase. In further embodiments, the Runx family member is Runx1 or Runx3.

In some embodiments, the effectors are used in combinations of at least two effectors from all the above mentioned groups linked through 2A peptide or IRES.

Dead Cas9 as an Effector

In some embodiments, the effector is dead Cas9 (dCas9), a version of Cas9 that does not have catalytic activity but retains target-specific binding to DNA-based transcriptional modulators. For example, dCas9-repressor domain fusion protein represses a targeted gene, whereas dCas9-activation domain fusion protein activates a targeted gene. In this system, dCas9 fusion protein would be expressed in an inducible manner, to limit immunogenicity, while sgRNA or multiplex sgRNAs targeting multiple genes may be expressed in a constitutive manner through RNA polymerase III promoter or from the LTR of a lentiviral vector. Examples of transcriptional repression domains are KRAB or DNMT3A, while examples of transcriptional activation domains are VP16, VP64, VPR or SunTag-based activators. This system allows shifting of T cell phenotype and function to a desired state based on targeted upregulation and/or downregulation of genes involved in the function of engineered immune cells. Such a system is feasible only in an all-in-one system.

Chimeric Antigen Receptors

The invention relates to compositions and methods for treating a disease. In some embodiments, the disease is a cancer, including but not limited to hematologic malignancies and solid tumors. The invention also encompasses methods of treating and preventing a disease, such as certain types of cancer, including primary and metastatic cancer, as well as cancers that are refractory or resistant to conventional chemotherapy. The methods comprise administering to a patient in need of such treatment or prevention a therapeutically or prophylactically effective amount of a T cell transduced to express a chimeric antigen receptor (CAR). Provided are compositions and methods comprising a CAR. CARs are molecules that combine antibody-based specificity for a desired antigen (e.g., tumor antigen) with a T cell receptor-activating intracellular domain to generate a chimeric protein that exhibits a specific anti-tumor cellular immune activity.

In some embodiments of any of the methods above, the methods result in a measurable reduction in tumor size or evidence of disease or disease progression, complete response, partial response, stable disease, increase or elongation of progression free survival, increase or elongation of overall survival, or reduction in toxicity.

In one embodiment, the CAR of the invention comprises an antigen binding domain, a transmembrane domain, and an intracellular signaling domain. In some embodiments, the intracellular signaling domain comprises one or more costimulatory signaling domains and an immunoreceptor tyrosine activation motif-containing signaling domain. In further embodiments, the immunoreceptor tyrosine activation motif-containing signaling domain comprises a CD3 zeta signaling domain.

In some embodiments, the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

The CAR of the invention can be engineered to comprise an extracellular domain having an antigen binding domain that targets tumor antigen fused to an intracellular signaling domain comprising the T cell antigen receptor complex zeta chain (e.g., CD3 zeta). An exemplary tumor antigen B cell antigen is CD19 because this antigen is expressed on malignant B cells. However, the invention is not limited to targeting CD19. Rather, the invention includes any tumor antigen binding moiety. In some aspects, CARs comprise: (1) a single-chain variable fragment (scFv) targeting a clinically-relevant antigen derived from a monoclonal antibody, (2) a transmembrane domain, (3) one or more costimulatory domains, and (4) an ITAM-containing signaling domain such as CD3-zeta.

In one embodiment, the CAR of the invention comprises a CD137 (4-1BB) signaling domain. For example, inclusion of the CD137 (4-1BB) signaling domain significantly increased CAR mediated activity and in vivo persistence of CAR T cells compared to an otherwise identical CAR T cell not engineered to express CD137 (4-1BB). However, the invention is not limited to a specific CAR. Rather, any CAR that targets a desired antigen, for example a tumor antigen, can be used in the present invention. Compositions and methods of making and using CARs have been described in PCT/US11/64191, which is incorporated by reference in its entirety herein.

The present invention provides a T cell genetically modified to express a chimeric antigen receptor (CAR) comprising an extracellular and intracellular domain. Compositions and methods of making CARs have been described in PCT/US11/64191, which is incorporated in its entirety by reference herein.

The extracellular domain comprises a target-specific binding element otherwise referred to as an antigen binding domain. In some embodiments, the extracellular domain also comprises a hinge domain. In one embodiment, the intracellular domain comprises a costimulatory signaling domain and a CD3 zeta chain portion. The costimulatory signaling domain refers to a portion of the CAR comprising the intracellular domain of a costimulatory molecule. Costimulatory molecules are cell surface molecules other than antigen receptors or their ligands that are required for an efficient response of lymphocytes to antigen.

Between the extracellular domain and the transmembrane domain of the CAR, or between the cytoplasmic domain and the transmembrane domain of the CAR, there may be incorporated a spacer domain. In this context, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

The present invention includes retroviral and lentiviral vector constructs expressing a CAR that can be directly transduced into a cell. The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyA tail, typically 50-2000 bases in length. RNA so produced can efficiently transfect different kinds of cells. In one embodiment, the template includes sequences for the CAR.

Preferably, the CAR comprises an extracellular domain, a transmembrane domain and a cytoplasmic domain. The extracellular domain and transmembrane domain can be derived from any desired source of such domains. In some instances, the hinge domain of the CAR of the invention comprises the CD8a hinge domain.

In one embodiment, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen moiety domain in the CAR of the invention include those associated with viral, bacterial and parasitic infections, autoimmune disease and cancer cells.

In one embodiment, the CAR of the invention can be engineered to target a tumor antigen of interest by way of engineering a desired antigen binding domain that specifically binds to an antigen on a tumor cell.

Tumor antigens are proteins that are produced by tumor cells that elicit an immune response, particularly T-cell mediated immune responses. The selection of the antigen binding domain of the invention will depend on the particular type of cancer to be treated. Tumor antigens are well known in the art and include, for example, a glioma-associated antigen, carcinoembryonic antigen (CEA), (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2 and mesothelin. The antigens discussed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20) have been used as targets for passive immunotherapy with monoclonal antibodies with limited success.

In some embodiments, the antigen is CD22, CD123, CD33, CD79b, ROR-1, CAIX, mesothelin, CMET, CD70, CLL-1, IL1RA, CD38, BCMA, CS-1, MUC1, CD2, CD5, CD7, CD30, CCR4, CD4, CD8, CD3, CD37, NKIG2D or FLT-3.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen binding domain portion of the CAR targets an antigen that includes but is not limited to CD19, CD20, CD22, ROR1, Mesothelin, CD33/IL3Ra, c-Met, PSMA, Glycolipid F77, EGFRvIII, GD-2, NY-ESO-1 TCR, MAGE A3 TCR, IL13Rα2 and the like.

Depending on the desired antigen to be targeted, the CAR of the invention can be engineered to include an appropriate antigen-binding moiety specific to the desired target.

The antigen-binding domain can be any domain that specifically binds to the antigen including but not limited to monoclonal antibodies, polyclonal antibodies, synthetic antibodies, human antibodies, humanized antibodies, and fragments thereof. In some instances, it is beneficial for the antigen-binding domain to be derived from the same species in which the CAR will ultimately be used. For example, for use in humans, it may be beneficial for the antigen-binding domain of the CAR to comprise a human antibody or fragment thereof. Thus, in one embodiment, the antigen biding domain portion comprises a human antibody or a fragment thereof. Alternatively, in some embodiments, a non-human antibody is humanized, where specific sequences or regions of the antibody are modified to increase similarity to an antibody naturally produced in a human.

In one embodiment of the present invention, a plurality of types of CARs is expressed on the surface of a T cell. The different types of CAR may differ in their antigen-binding domain. That is, in one embodiment, the different types of CARs each bind a different antigen. In one embodiment, the different antigens are markers for a specific tumor. For example, in one embodiment, the different types of CARs each bind to a different antigen, where each antigen is expressed on a specific type of tumor. Examples of such antigens are discussed elsewhere herein.

In some embodiments, the CAR comprises a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, ICOS. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact signaling domain as long as it transduces the effector function signal. The term intracellular signaling domain is thus meant to include any truncated portion of the intracellular signaling domain sufficient to transduce the effector function signal.

In one embodiment, the effector function of the cell is dependent upon the binding of a plurality of types of CARs to their targeted antigen. For example, in one embodiment, binding of one type of CAR to its target is not sufficient to induce the effector function of the cell.

Primary cytoplasmic signaling sequences regulate activation of the TCR/CD3 complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In one embodiment, the cytoplasmic domain of the CAR comprises the CD3-zeta signaling domain by itself or combined with any other desired cytoplasmic domain(s) useful in the context of the CAR of the invention. For example, the cytoplasmic domain of the CAR can comprise a CD3-zeta chain portion and a costimulatory signaling domain. The costimulatory signaling domain refers to a portion of the CAR comprising the intracellular domain of a costimulatory molecule.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain comprises the signaling domain of CD3-zeta. In another embodiment, the cytoplasmic domain comprises the signaling domain of CD3-zeta and the signaling domain of 4-1BB. In one embodiment of the present invention, a plurality of types of CARs is expressed on a cell, where the different types of CAR may vary in their cytoplasmic domain. In one embodiment, at least one type of CAR comprises the CD3 zeta domain, while at least one type of CAR comprises a costimulatory domain, for example the 4-1BB domain. However, the different types of CARs are not limited by any particular cytoplasmic domain.

In one embodiment, the T cell genetically modified to express a CAR is further engineered or edited using CRISPR/Cas.

Extracellular Domain

In another embodiment, the extracellular domain can include any portion of an antibody that binds to antigen including, but not limited to, the antigen binding domain of a synthetic antibody, human antibody, humanized antibody, single domain antibody, single chain variable fragments, and fragments thereof. In some instances, it is beneficial for the extracellular domain to be derived from the same species in which the chimeric membrane protein will ultimately be used in. For example, for use in humans, it may be beneficial for the extracellular domain of the chimeric membrane protein to comprise a human antibody or fragment thereof. Thus, in one embodiment, the extracellular domain portion comprises a human antibody or a fragment thereof.

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Alternatively, in some embodiments, a non-human antibody is humanized, where specific sequences or regions of the antibody are modified to increase similarity to an antibody naturally produced in a human. In one embodiment, the antigen binding domain portion is humanized.

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A “humanized” antibody retains a similar antigenic specificity as the original antibody. However, using certain methods of humanization, the affinity and/or specificity of binding of the antibody for human CD3 antigen may be increased using methods of “directed evolution,” as described by Wu et al., J. Mol. Biol., 294:151 (1999), the contents of which are incorporated herein by reference herein in their entirety.

In one embodiment, the antibody is a synthetic antibody, human antibody, a humanized antibody, single chain variable fragment, single domain antibody, an antigen binding fragment thereof, and any combination thereof.

Intracellular Domain

The intracellular domain or cytoplasmic domain comprises a costimulatory signaling domain. The costimulatory signaling domain refers to an intracellular domain of a costimulatory molecule. Costimulatory molecules are cell surface molecules other than antigen receptors or their ligands that are required for an efficient response of lymphocytes to antigen.

Other Domains of the Chimeric Antigen Receptor

Between the extracellular domain and the transmembrane domain of the chimeric antigen receptor, or between the cytoplasmic domain and the transmembrane domain of the chimeric antigen receptor, there may be incorporated a spacer domain.

In some embodiments, the chimeric membrane protein further comprises a transmembrane domain. In some embodiments, the chimeric membrane protein further comprises a hinge domain. In one embodiment, the RNA encoding the chimeric membrane protein further comprises a transmembrane and hinge domain, such as a CD28 transmembrane domain and a CD8alpha hinge domain.

Therapy

As a first form of gene transfer therapy to gain approval by FDA, immunotherapy with chimeric antigen receptor (CAR) T cells specific for the CD19 B lymphocyte molecule revolutionized treatment of certain blood cancers. CAR T cells kill large amounts of tumor cells in a short time. This antigen-specific killing can be associated with an uncontrolled release of inflammatory mediators and general disruption of homeostasis referred to as cytokine release syndrome (CRS). This can cause life-threatening adverse events and if CRS is manifested in the central nervous system (CNS), this can lead to fatal neurological toxicities. Systemic administration of Tocilizumab (a monoclonal antibody that blocks the interleukin-6 receptor (IL-6R)) that blocks the IL-6 signaling axis is currently used to manage such adverse events. Early detection and administration are needed to prevent damage, but controlling neurological toxicities is inefficient because the blood-brain barrier prevents entry of the drug into the CNS. The use of CD19 CARs targeting hematological malignancies (or CAR T cells targeting other antigens) in combination with in-situ secretion of an engineered antibody that blocks IL-6 signaling and/or additional inflammatory mediators such as TNFalpha or IL-1beta will likely increase the safety of cancer immunotherapy in which CRS triggered by adoptive cell therapy is a major problem. Without wishing to be bound by theory, autonomous and prompt delivery of the drug by therapeutic cells themselves might suppress CRS even before it becomes clinically detectable. Since therapeutic cells that enter CNS are responsible for neurological toxicities, this could solve the issue of poor CNS penetrance with Tocilizumab (or any other antibody that blocks IL-6 signaling or signaling by other inflammatory cyto- and chemokines), which represents a paradigm changing approach to control toxicities related to successful immunotherapy.

The engineered cells described herein may be included in a composition for therapy either separately or in combination. The composition may include a pharmaceutical composition and further include a pharmaceutically acceptable carrier. A therapeutically effective amount of the pharmaceutical composition comprising the engineered cells described herein may be administered.

In some embodiments, the engineered cell may be administered with a second agent. In some embodiments, the engineered cell and the second agent are administered to the patient simultaneously or sequentially. In some embodiments, the engineered cell and the second agent are administered to the patient in the same composition. In some embodiments, the engineered cell and the second agent are administered to the patient as separate compositions.

In one aspect, the invention includes a method for stimulating a T cell-mediated immune response to a target cell or tissue in a subject comprising administering to a subject an effective amount of an engineered cell. The engineered cells described herein may be administered to induce lysis of the target cell or tissue, such as where the induced lysis results from antibody-dependent cell-mediated cytotoxicity (ADCC).

In another aspect, the invention includes a method for adoptive cell transfer therapy comprising administering a population of engineered cells described herein to a subject in need thereof to prevent or treat an immune reaction that is adverse to the subject.

In yet another embodiment, a method of treating a disease or condition associated with enhanced immunity in a subject comprising administering a population of engineered cells described herein to a subject in need thereof.

The modified T cells generated as described herein possess T cell function. Further, the engineered cells described herein can be administered to an animal, preferably a mammal, even more preferably a human, to suppress an immune reaction, such as those common to autoimmune diseases such as diabetes, psoriasis, rheumatoid arthritis, multiple sclerosis, graft versus host disease (GVHD), enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease. In one aspect, the invention includes treating a condition, such as an autoimmune disease, in a subject, comprising administering to the subject a therapeutically effective amount of a pharmaceutical composition comprising a population of modified T cells.

In some embodiments for the treatment of an autoimmune disease, or any condition in which a diminished or otherwise inhibited immune response, especially a cell-mediated immune response, is desirable to treat or alleviate the disease, the modified T cell or the engineered immune cell or precursor cell thereof may be a Treg or a myeloid derived suppressor cell (MDSC). In some embodiments, the effector is an immunosuppressive molecule, such as IL-10, or an anti-inflammatory blocker, such as anti-TNF, anti-IL1 or anti-IL6.

Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The engineered cells generated as described herein can also be modified and used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In another embodiment, the engineered cells described herein may be used for the manufacture of a medicament for the treatment of an immune response in a subject in need thereof.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous or allogenic with respect to the subject undergoing therapy.

The administration of the cells or compositions of the invention may be carried out in any convenient manner known to those of skill in the art. The cells or compositions of the present invention may be administered to a subject by injection, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

The cells or compositions described herein can also be administered using any number of matrices. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized, as such these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

CRISPR

In some embodiments of the invention, an endogenous TCR of the engineered cell has been knocked out using a CRISPR-related system. In some embodiments, the CRISPR-related system is a CRISPR/Cas9 system.

Also provided are some embodiments with a CRISPR-related system-mediated knock-in of the designed expression cassette into the TCR or HLA (or other relevant loci) to make the introduced receptor-mediated signaling construct the exclusive activator of the system and to make engineered cells universal, respectively. In some embodiments, the designed expression cassette is encoded in a donor template. In some embodiments, the donor template is encoded in a viral vector. In some embodiments the viral vector is an adeno-associated viral vector. In further embodiments, the adeno-associated viral vector is AAV6. In some embodiments, the CRISPR-related system is a CRISPR/Cas9 system.

In some embodiments, such a system may prevent, decrease or treat recombinant TCR-mediated graft-versus-host disease.

Provided is an engineered immune cell or engineered immune cell precursor cell, comprising: an insertion in a gene locus, wherein the insertion comprises a first polynucleotide comprising a constitutive promoter operably linked to a nucleic acid encoding at least one transgene, wherein one of the at least one transgenes encodes a receptor or receptor subunit, a receptor fusion protein or a fluorescent marker; and a second polynucleotide comprising an inducible promoter operably linked to a nucleic acid encoding an effector. In some embodiments, the transgene encodes a receptor fusion protein.

In some embodiments, the receptor fusion protein comprises an antigen-specific synNotch receptor fusion protein and an optional reporter. In some embodiments, the receptor fusion protein further comprises a 2A peptide between the antigen-specific synNotch receptor and the reporter. In some embodiments, the reporter is BFP2. In further embodiments, the antigen-specific synNotch receptor fusion protein is a HER2-specific synNotch receptor fused to Gal4-VP64. In further embodiments, binding of the synNotch receptor to its target antigen HER2 induces Notch1 cleavage which releases the transcription factor Gal4-VP64 from the fusion protein.

In some embodiments, the transgene is a receptor or receptor subunit. In some embodiments, the receptor or receptor subunit is a cytokine receptor or a cytokine receptor subunit. In further embodiments, the cytokine receptor subunit is IL-6Ra.

In some embodiments, the receptor fusion protein is a CAR comprising an extracellular antigen-binding domain capable of specifically binding a cognate antigen and an intracellular signaling domain.

In some embodiments, the transgene is a fluorescent marker. In further embodiments, the fluorescent marker is mCherry.

In some embodiments, the first polynucleotide, the second polynucleotide or both the first and the second polynucleotides comprise an insulator sequence or a linker sequence.

In some embodiments, the insertion is encoded in a donor template. In some embodiments, the donor template is encoded in a viral vector. In some embodiments, the gene locus encodes a TCR subunit, an HLA or an immune checkpoint molecule. In further embodiments, the immune checkpoint molecule is PD-1, TIM3, LAG3, CTLA4, 2B4, CD160 or CD5.

In some embodiments the viral vector is an adeno-associated viral vector. In further embodiments, the adeno-associated viral vector is AAV6. In some embodiments, an endogenous TCR or HLA (Human leukocyte antigen) or immune checkpoint molecule (such as PD-1, TIM3, LAG3, CTLA4, 2B4, CD160, CD5) or other molecule that negatively affects immune cell function of interest of the engineered cell has been knocked out using a CRISPR system, for example a CRISPR/Cas9 system, with non-homologous end joining or homologous recombination where designed construct serves a donor template molecule.

In some embodiments, the gene locus encodes TCR or HLA (Human leukocyte antigen) or immune checkpoint molecule (such as PD-1, TIM3, LAG3, CTLA4, 2B4, CD160, CD5) or other molecule that negatively affects immune cell function of interest of the engineered cell.

In some embodiments, the insertion in a gene locus is mediated by a CRISPR-related system. In further embodiments, the CRISPR-related system is CRISPR/Cas9.

Pharmaceutical Compositions

Pharmaceutical compositions of the present invention may comprise an engineered immune cell as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the modified T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 ml to 400 ml. In certain embodiments, T cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of T cells.

In certain embodiments of the present invention, cells expanded and modified using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1—System Design

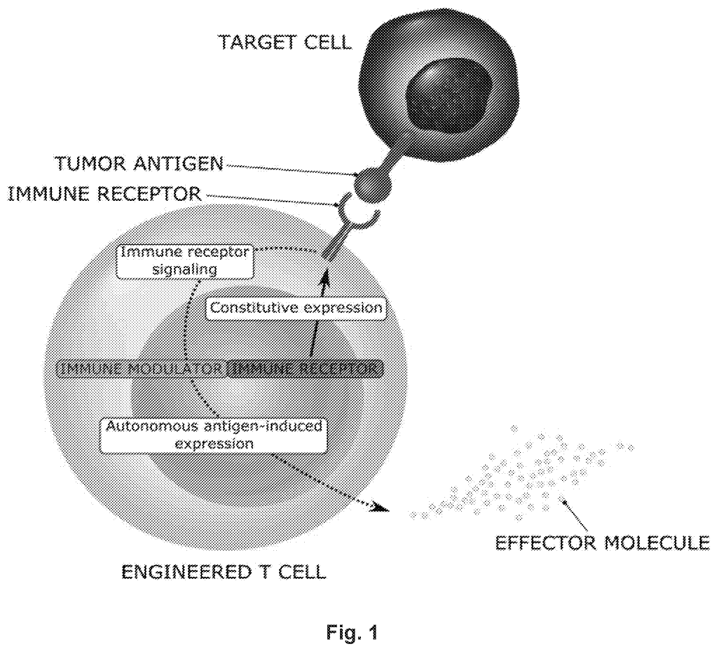

In the presented invention the designing and testing of a novel first-of-its-kind lentiviral vector system with unique properties that allowed for a simultaneous constitutive and inducible expression within a single lentiviral vector was demonstrated (FIG. 1). A constitutive part of the system enabled strong constitutive promoter driven expression of immune receptor such as CAR or TCR, while inducible part or the system provided autonomous expression of effector immunomodulatory molecules triggered by introduced CAR/TCR-specific or other tumor microenvironment characteristic signaling. More specifically, the system presented in this disclosure sensed endogenous CAR/TCR or other tumor microenvironment characteristic signaling through the activation of specific promoters introduced with the regulatory part of the lentiviral vector and rewired this signaling to the expression of the selected immunomodulatory molecules, which should in principle be restricted to the local tumor microenvironment.

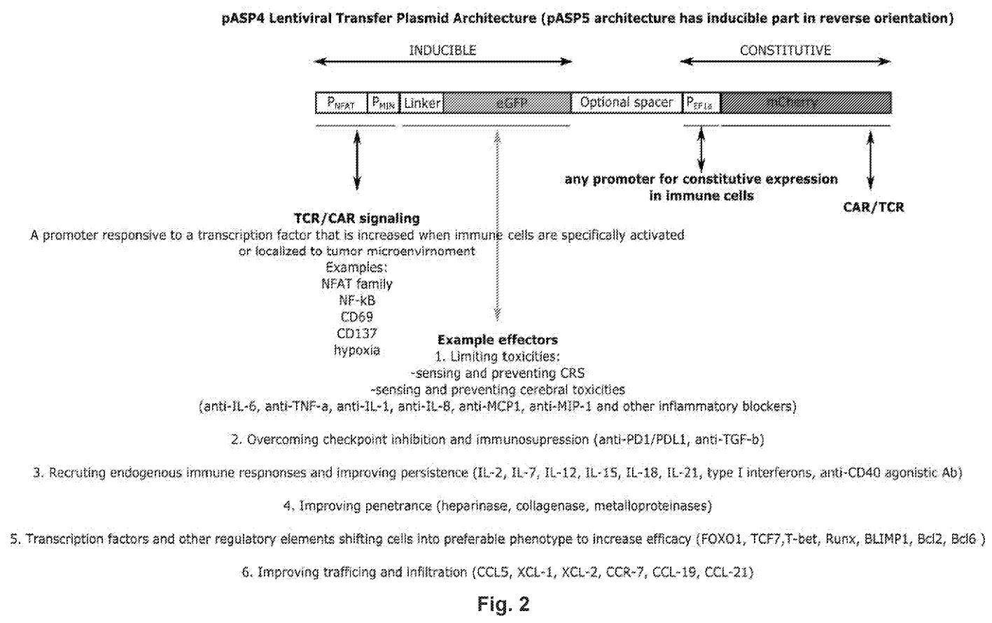

For this purpose different architectures of unique lentiviral plasmids were prepared, which comprised different inducible promoters including a synthetic NFAT promoter, CD69 promoter, CD137 promoter or synthetic hypoxia responsive element (HRE) promoter linked with best in class minimal promoter (P_{MIN}) validated in our previous studies (Smole, A., Lains̆c̆ek, D., Bezeljak, U., Horvat, S. & Jerala, R. A Synthetic Mammalian Therapeutic Gene Circuit for Sensing and Suppressing Inflammation. Mol. Ther. 25, 102-119 (2017)), which enabled low basal leakage and high expression when induced through NFAT promoter. Inducible part was connected with the constitutively expressed module in the same lentiviral vector in different architectures whether both driving expression in the same (pASP4 architecture) or in the opposite (pASP5 architecture) directions (FIG. 2).

### Example 2—Autonomous NFAT-Driven Expression of eGFP and Constitutive Expression of mCherry from a Single Lentiviral Vector in Jurkat Cell Line

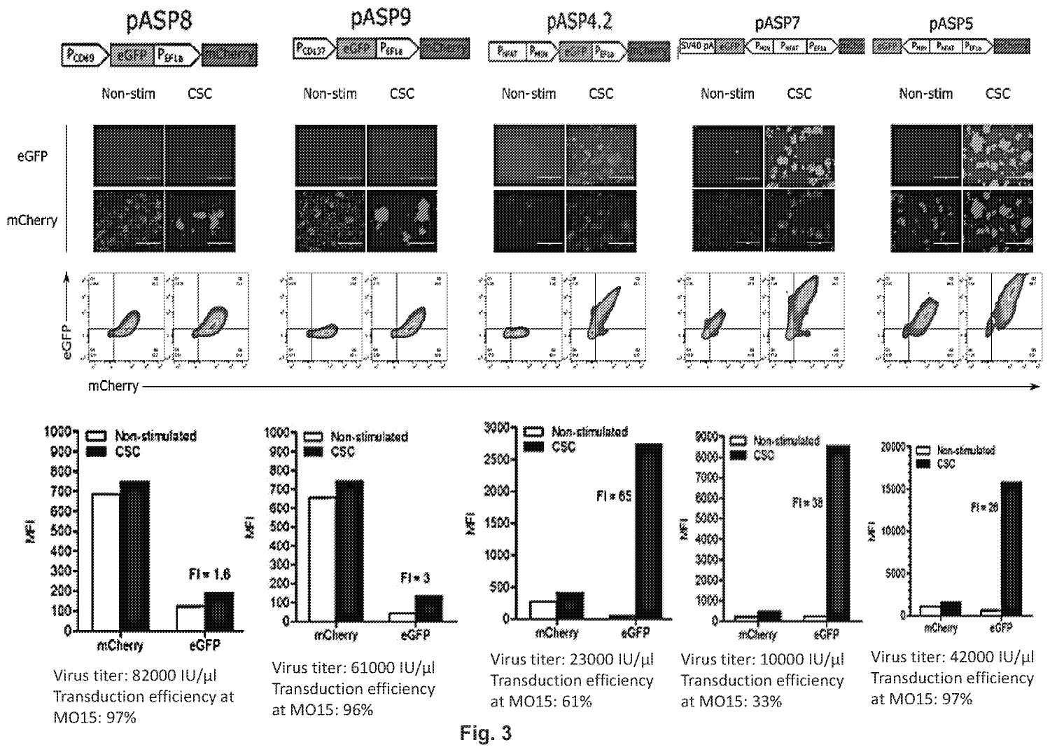

As a proof of principle, the lentiviral system described herein enabled constitutive production of mCherry and inducible expression of eGFP in transduced Jurkat cells as measured by fluorescence microscopy and flow cytometry (FIG. 3). Cells were non-specifically activated with CSC (ionomycin and phorbol myristate acetate) which short-circuits Ca²⁺ TCR signaling in T cells. Different promotor elements that are active specifically in the activated T cells were tested. Architectures pASP8 and pASP9 comprised CD69 and CD137 promoters respectively. ON/OFF ratio in both examples was low (from 1.5-3) and additionally, CD69 promoter shown substantial leakiness. Architecture pASP4.2, comprising synthetic NFAT promoter linked to the optimized minimal promoter (P_{MIN}) enabled medium ON state while it remained silent in the absence of the trigger signal, which is attributed to the careful selection of NFAT binding sites and P_{MIN}, resulting in 65-fold induction. This configuration is of high value when no leakiness is acceptable due to potential toxicities, for example when using immune modulators such as cytokines. Architecture pASP5 differs from pASP4.2 in the orientation of the inducible part, which is placed in the reverse orientation, back-to-back with the constitutively expressed part of the vector. This architecture resulted in substantially higher ON state but also higher leakiness (fold induction of around 25), while virus titer was also significantly better. The latter is attributed to the orientation of consecutive NFAT binding sites which likely interfered with virus mRNA production and was less adverse when placed in the opposite direction to the virus mRNA transcription. pASP5 configuration is suitable for the systems where some basal leakiness is not expected to pose serious safety concerns and high ON state production is required, which would be the case when using therapeutic antibodies such as inflammatory cytokines blockers or checkpoint inhibitors. pASP7 architecture is the same as pASP5 with the only difference of SV40 poly adenylation (pA) signal added to the 3′ end of eGFP to increase its expression. While increasing eGFP expression, this configuration also decreased virus titer significantly, since SV40 pA even if placed in the reverse orientation severely interferes with virus mRNA production.

Interestingly, back-to-back orientation of inducible and constitutive promoters influenced expression in both directions as observed in constructs pASP5 and pASP7, where constitutively expressed mCherry increased after stimulation and leakage of inducible eGFP was higher than expected in non-stimulated cells. This phenomenon and its use to our advantage are discussed in more detail below.

### Example 3—Autonomous NFAT-Driven Expression of eGFP and Constitutive Expression of mCherry from a Single Lentiviral Vector in Primary Human T Cells

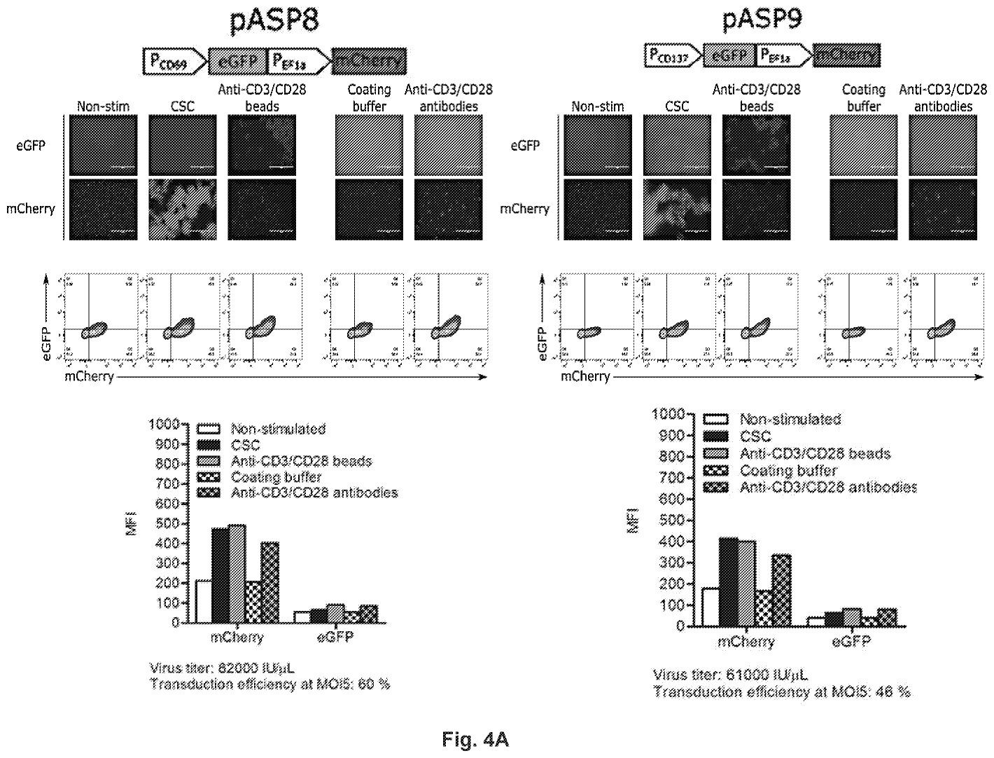

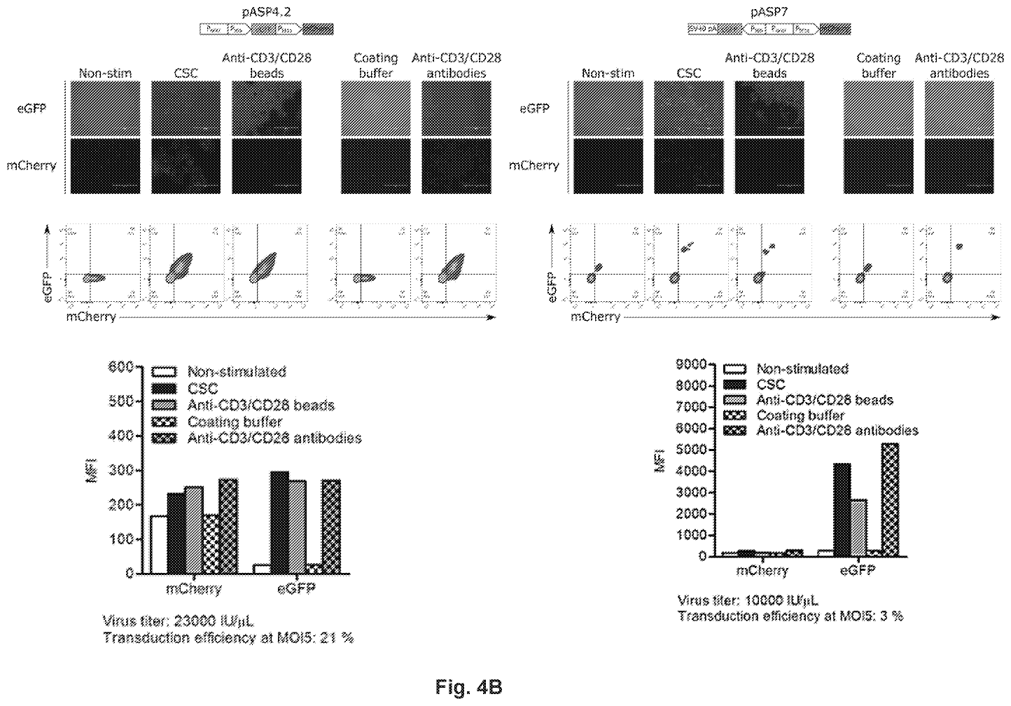

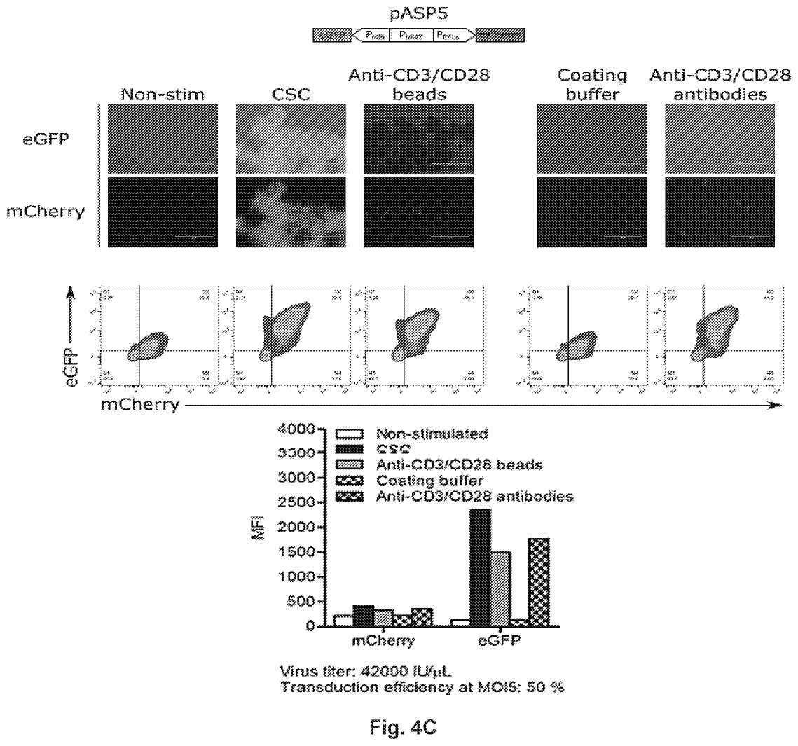

To demonstrate that the developed system works also in therapeutically used primary T cells with more relevant inducers resampling TCR signalling, primary donor-derived T cells were activated, transduced with constructs pASP8, pASP9, pASP4.2, pASP7 and pASP5 and expanded according to the standard protocol. Engineered cells were rested for 14 days to return activation state to the basal levels and then stimulated with CSC, anti-CD3/CD28 beads and anti-CD3/CD28 antibodies-coated plates. Results recapitulated our findings from Jurkat cell lines, showing reliable constitutive expression of mCherry and roboust inducibility of eGFP in constructs pASP4.2 and pASP5 as measured by fluorescence microscopy and flow cytometry (FIG. 4). Imprtantly, physiologicaly relevant TCR-mediated signaling induced by anti-CD3/CD28 beads and anti-CD3/CD28 antibodies-coated plates resulted in similar activation amplitude as in the case of CSC that represented maximal system's activity. System showed tight regulation with no detectable (pASP4.2) or very low (pASP5) background activity in the absence of activation signal.

### Example 4—Capacity of the Activated System

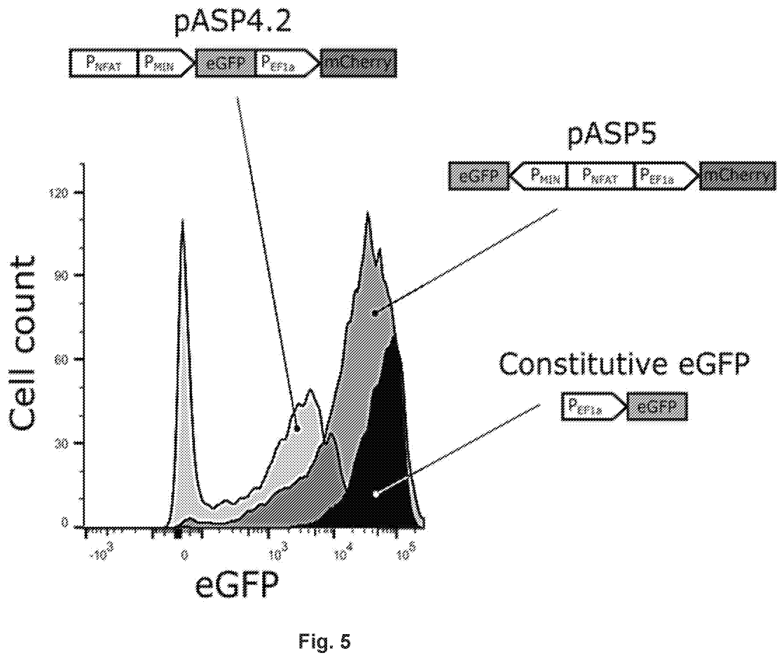

In one aspect, the purpose of the developed system is to produce various effectors in sufficient amounts to mediate therapeutic effect. To validate the capacity of the developed system, median fluorescence intensity was determined as a measure of the amount of eGFP produced in an inducible manner. pASP5 as a higher expressing architecture produced comparable levels to constitutively expressed eGFP, meaning that the system's capacity was high. pASP4.2 version, as expected, expressed at lower amounts, which recapitulates findings shown in previous figures and supports the rationale for using these two architectures according to the specific needs of the respective therapeutic molecule (FIG. 5).

### Example 5—Reversibility of the System

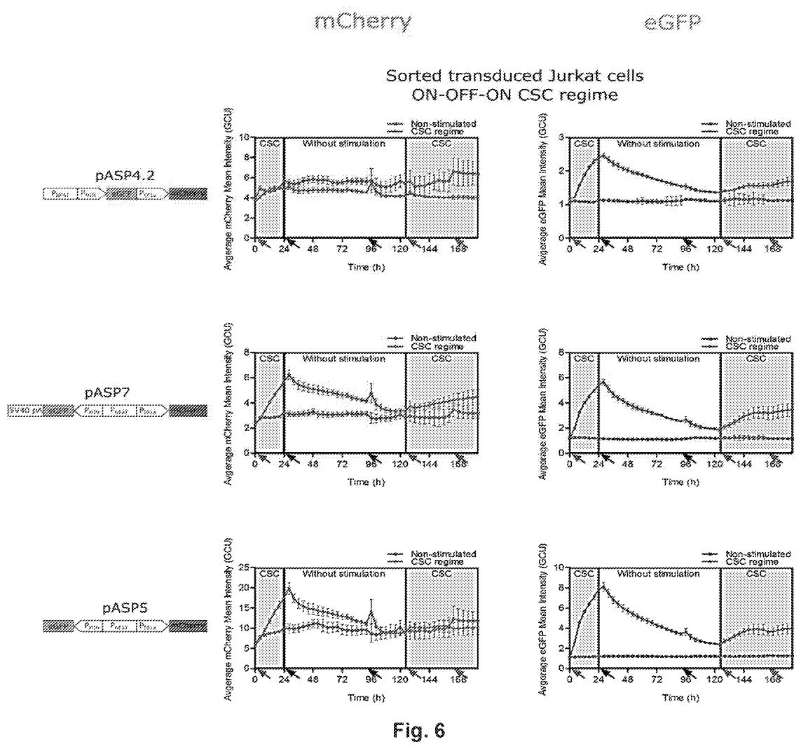

The reversibility of the presented system is evidenced by the data presented herein and its capability of responding to multiple rounds of stimulation, which is how a native immune system works. This is relevant if the cancer resides in different sites of the body and same cell encounters target cells many times or if the engineered cells are exposed to the same target multiple times (for example in reoccurring disease). Using IncuCyte technology to monitor fluorescence in a real-time manner, pASP4.2, pASP5 and pASP7 transduced Jurkat cells were stimulated with CSC for 24 h and observed rapid eGFP expression, which was detectable already after 4 h revealing rapid kinetics of the developed inducible system (FIG. 6A). After washing step, eGFP decreased gradually over 4 days, which is limited to the molecule half-life, and after re-stimulation at day 5, it increased again, demonstrating a reversible nature of the system. This was shown also later with a secreted molecule to more accurately capture the kinetics and to show that the system resets quickly after stimulus removal and returns to the initial maximum activation levels after second stimulation (section “On demand tailored and reversible expression of hIL-12 to increase efficacy of engineered primary T cells”). mCherry as a constitutive part remained expressed regardless of the stimulation, however in the pASP5 and pASP7 back-to-back orientation its expression was increased in stimulated cells, which is in agreement with results shown in previous figures and also with published studies showing different levels of bidirectional activity of certain promoter elements (Amendola, M., Venneri, M. A., Biffi, A., Vigna, E. & Naldini, L. Coordinate dual-gene transgenesis by lentiviral vectors carrying synthetic bidirectional promoters. Nat. Biotechnol. 23, 108-116 (2005)). This phenomenon probably increased basal expression of inducible part in pASP5 orientation as well, since EF-1alpha promoter influences NFAT-P_{MIN} inducible part. On the other hand, NFAT transcriptional factors, recruited to consecutive NFAT binding sites in synthetic promoter in stimulated T cells increased the strength of already strong EF-1alpha promoter even further as shown by increase in mCherry expression after stimulation. This is useful feature because the system behaves in a feed-forward loop manner, meaning that CAR expression would be increased in a positive-feedback loop after initial recognition but in an antigen-dependent manner. This could increase sensitivity of the system in the tumor microenvironment where cancer cells downregulate target antigen, whereas sensitivity would return to normal levels when antigen in no more present.

In this Example, a functionally connected regulatory unit has been shown in the forward orientation but the same configuration is used when inducible and constitutive modules are placed back to back in the opposite direction, where inducible module is placed in reverse and constitutive module in forward orientation. Also provided herein is the 5′-3′ sequence and annotation of this exact part, because this orientation is often used because it confers better virus titer and hence higher clinical relevance:

Preceded in reverse orientation by immune modulator starting with a start codon—

(SEQ ID NO: 3)
GGTGGCTTTACCAACAGTACCGGAATGCCCCCGGGCTGGAAGTCGAGCT

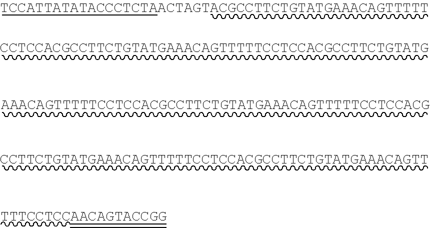

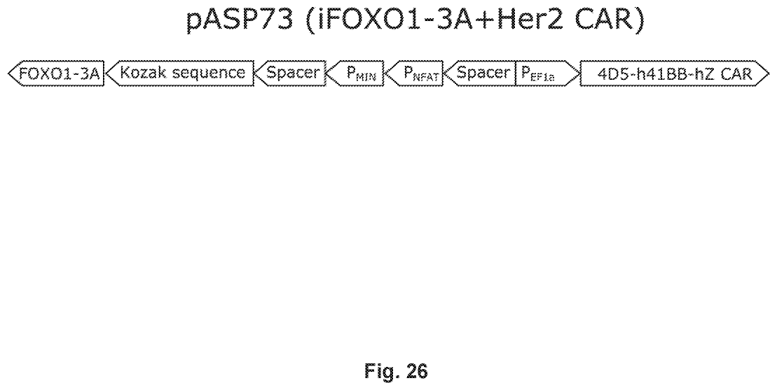

Followed in forward orientation by strong constitutive promoter such as EF1alpha—followed in forward by immune receptor such as CAR or TCR starting with start codon.
Kozak sequence: bold
Spacer for optimal distance between transcription and translation start site: italics
Minimal promoter: underlined
Synthetic NFAT promoter: wave underline Spacer for cloning purposes: double underline
   A schematic representation is shown in FIG. 26 where orientation of the arrow means direction (forward or reverse) and entire sequence as exemplified by FOXO1-3A as immune modulator and 4D5 Her2-targeting CAR (4-1BB+CD3Z). The functionally connected regulatory unit from above is marked in bold in the sequence below (SEQ ID NO: 4):

TTAGCCGGAAACCCAGCTGTGTGTGGTGGTTTTCACGCTGTGAGGGAAGC
TCTGGTTGGGCAGCACGTTATCGAAGTTGAAGTCCAGGGTATCGCCGTCC
ATCAGGTCGTTGCGGATGATGGATTCCATGTCGCAGTCCAGTCTCTCGAT
GAACATGCCGTCCAGATCAGAGGGCAGCTTCTCTTGGTGCAGCAGGCCCA
TTCTGCCGTAGCCATTACAGCTGGACACGCTGGAGTAGCCGCCGAGGGCA
GACATCTGCATAGGATGAGGCAGTGGGACCTGCACAGGGGTTTTCACTTG
GGTCAGCCGGTTCATGCCGCTGGTATGAGGCATGGTGGACACGGTGTGAG
GCAGAGGTCTGCCATTAACGGCGCTTGTCTGCTGGGCATGTCCAGGGTGT
GTGTGGCTGCTGGGGTTCATCATCTTGTTGTGGCTGGCCTGAGAGCCGTA
TGTGCTCATCACGCTATTGGGGCCCATCATCACGTTCTGGCCCAGCACTC
TACTATTAGGCTGGGCCACGCCAGGGTCCACAGGTGTCATGATGTCGTTG
TGAGGAGGGCTGTCAGAGGTCAGCAGCTCTTTCAGCAGTCCAGGGGCACA
ATTGTACTGGCTCATGCCGCCGTAGGAGGACTTGTTGTCCTGCAGGGTCT
GAATGGGCATCTGTGGCAGAGGGCTCATGCTGCTCTGGCCGTAGGTGTAC
TTCTGGTAGTTAGGAGATGGGCTGTTCAGGCTTGTGTTAGGAGGGGCGAA
GCTGTAGCAAGGGGTCTGCTGCATCATGGTGCCGGGGCTGCTTTGTGTAG
ACACGGTCAGGCTGGTAGGGCTGCTCAGCAGATTCAGGTTGTCCAGCAGG
TTTTCCATGTTCTCGGGGTTGCTGATCTCGCTCAGGCTTGGCAGTGTGCT
GGCCATTTTGGCGGCGCTTGGAGGATACACCATGCTGTGCACGTCGCCTT
CTCCGAGATCATCCTGCTCGGTCATGATGGGGCTCAGTCTGCCAGAGATG
GTGCTGGCATTGGCGCTGGTTCTGGGTCTGAAGGTGGACCAGTTGTCGAA
GTCGTCGTTGCTGTGAGAGCCAGGAGAGGCGGGCCACTTAGAAAACTGGC
TGCCAGGAGAATCGCCAGCGCCTTCTTGTCCAGACTGCAGAGAGGCCTTC
TTCTTGGCGGCTCTGCTTCTGCTCTTGGCGAACTTGGAGTTGTTGTCCAT
GGCAGCGGCCCTTCTACGAGGAGACTTGCCGCTCTTGCCGCCTTCGGGAT
TCAGCATCCACCAAGAGGACTTGCCGGTGCCTTCATTCTGCACCCGGATG
AACTTGCTGTGCAGGGACAGATTGTGCCGGATGCTGTTCTTCCAGCCGGC
GGAGCTATTGCTGTCGCCCTTGTCCTTGAAGTAGGGCACGCTCTTGACCA
TCCACTCGTAGATCTGGCTCAGGGTCAGTCTCTTCTCGGCGCTGCTCTCA
ATGGCCTTGGTGATCAGGTCGGCGTAGCTCAGATTGCCCCAGGCGTTTCT
TCTGCTGGAGCTGCTCTTTCTAGGCTGTCCAGCAAGTGGTCCAGCGGCAG
CTGGAGGAACAGGAGGATGCTGAGACAGAGGTCCAGGTGGAGGTGGTTGA
GGTGGAGCAGGGTGCAGACATCCGGCCTCAGGTCCCTGAAAATCGCCGCA
AAGTCCACCTGTAGCAGCAGCTGCGGCTGCGGCGGCAACAGCTGCAGCCA
CAGATCCAGGAGCCTGAGGGAAGTCCTCGCTCTCTTCCAGCAGGCTCAGG
TTGCTCATGAAGTCGGCGGACACTGCAGCAGCAGAGGCAGAAGGCAGTCC
AGCAGCAGCATCAGGATTAGCGGCGGCAGATCCGCTAGGAGCAGGGCTAG
ATGTGGCGCTGTTGCTCTGGCTGAACTCAGGTCTAGGCAGAGGCCAAGCA
CAAGATCTAGGCCGAGGCAGGGGCTCGAAATCAGGGTCGATTTCCACCAC
CTGGGGAGCCTCGGCCATGGTGGCTTTACCAACAGTACCGGAATGCCCCC
GGGCTGGAAGTCGAGCTTCCATTATATACCCTCTAACTAGTACGCCTTCT
GTATGAAACAGTTTTTCCTCCACGCCTTCTGTATGAAACAGTTTTTCCTC
CACGCCTTCTGTATGAAACAGTTTTTCCTCCACGCCTTCTGTATGAAACA
GTTTTTCCTCCACGCCTTCTGTATGAAACAGTTTTTCCTCCACGCCTTCT
GTATGAAACAGTTTTTCCTCCAACAGTACCGGATGCATCGTGAGGCTCCG
GTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGT
AAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGC
AACGGGTTTGCCGCCAGAACACAGGTAAGTGCCGTGTGTGGTTCCCGCGG
GCCTGGCCTCTTTACGGGTTATGGCCCTTGCGTGCCTTGAATTACTTCCA
CCTGGCTGCAGTACGTGATTCTTGATCCCGAGCTTCGGGTTGGAAGTGGG
TGGGAGAGTTCGAGGCCTTGCGCTTAAGGAGCCCCTTCGCCTCGTGCTTG
AGTTGAGGCCTGGCCTGGGCGCTGGGGCCGCCGCGTGCGAATCTGGTGGC
ACCTTCGCGCCTGTCTCGCTGCTTTCGATAAGTCTCTAGCCATTTAAAAT
TTTTGATGACCTGCTGCGACGCTTTTTTTCTGGCAAGATAGTCTTGTAAA
TGCGGGCCAAGATCTGCACACTGGTATTTCGGTTTTTGGGGCCGCGGGCG
GCGACGGGGCCCGTGCGTCCCAGCGCACATGTTCGGCGAGGCGGGGCCTG
CGAGCGCGGCCACCGAGAATCGGACGGGGGTAGTCTCAAGCTGGCCGGCC
TGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTT
CCCGGCCCTGCTGCAGGGAGCTCAAAATGGAGGACGCGGCGCTCGGGAGA
GCGGGCGGGTGAGTCACCCACACAAAGGAAAAGGGCCTTTCCGTCCTCAG
CCGTCGCTTCATGTGACTCCACTGAGTACCGGGCGCCGTCCAGGCACCTC
GATTAGTTCTCGTGCTTTTGGAGTACGTCGTCTTTAGGTTGGGGGGAGGG
GTTTTATGCGATGGAGTTTCCCCACACTGAGTGGGTGGAGACTGAAGTTA
GGCCAGCTTGGCACTTGATGTAATTCTCCTTGGAATTTGCCCTTTTTGAG
TTTGGATCTTGGTTCATTCTCAAGCCTCAGACAGTGGTTCAAAGTTTTTT
TCTTCCATTTCAGGTGTCGTGATTCTAGAGCCACCATGGCCTTACCAGTG
ACCGCCTTGCTCCTGCCGCTGGCCTTGCTGCTCCACGCCGCCAGGCCGGA
CATTCAAATGACACAGAGTCCCTCATCCCTCAGTGCCAGCGTGGGCGATC
GGGTGACTATAACCTGCAGAGCTTCTCAGGACGTGAATACCGCTGTGGCG
TGGTACCAGCAGAAGCCAGGCAAAGCGCCTAAGCTTCTCATTTATAGTGC
CAGCTTCCTGTACTCAGGTGTTCCGTCTCGCTTTTCTGGAAGTAGAAGTG
GGACCGATTTCACATTGACGATCAGCAGCTTGCAGCCCGAAGATTTCGCC
ACCTACTACTGTCAGCAGCACTACACTACCCCACCGACATTTGGTCAAGG
CACAAAAGTAGAGATTAAACGCACTGGTTCCACCAGCGGGAGCGGGAAAC
CCGGCTCTGGGGAGGGGAGCGAGGTCCAGCTGGTGGAATCCGGGGGTGGT
CTTGTGCAGCCAGGAGGATCCTTGAGGTTGTCCTGCGCCGCAAGCGGCTT
TAACATCAAAGATACATACATCCATTGGGTCCGACAGGCCCCTGGAAAGG
GCCTGGAGTGGGTCGCCCGGATCTACCCAACTAACGGGTACACTCGCTAC
GCTGATAGCGTCAAGGGTCGGTTTACTATTTCTGCCGACACCTCAAAAAA
CACAGCCTACCTCCAGATGAACTCTCTCAGAGCTGAGGATACAGCCGTGT
ACTATTGCAGCCGGTGGGGAGGCGACGGGTTCTACGCTATGGATGTGTGG
GGGCAGGGCACACTGGTCACCGTGAGCTCATCCGGAACCACGACGCCAGC
GCCGCGACCACCAACACCGGCGCCCACCATCGCGTCGCAGCCCCTGTCCC
TGCGCCCAGAGGCGTGCCGGCCAGCGGCGGGGGGCGCAGTGCACACGAGG
GGGCTGGACTTCGCCTGTGATATCTACATCTGGGCGCCCTTGGCCGGGAC
TTGTGGGGTCCTTCTCCTGTCACTGGTTATCACCCTTTACTGCAAACGGG
GCAGAAAGAAACTCCTGTATATATTCAAACAACCATTTATGAGACCAGTA
CAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCC
CCGCGTACAAGCAGGGCCAGAACCAGCTCTATAACGAGCTCAATCTAGGA
CGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATG
AACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAG
TACAGCCACCAAGGACACCTACGACGCCCTTCACATGCAGGCCCTGCCCC
CTCGCTAA

### Example 6—Designer T Cells Respond Specifically to Antigen-Positive Cancer Cell Line and Retain CAR Mediated Killing

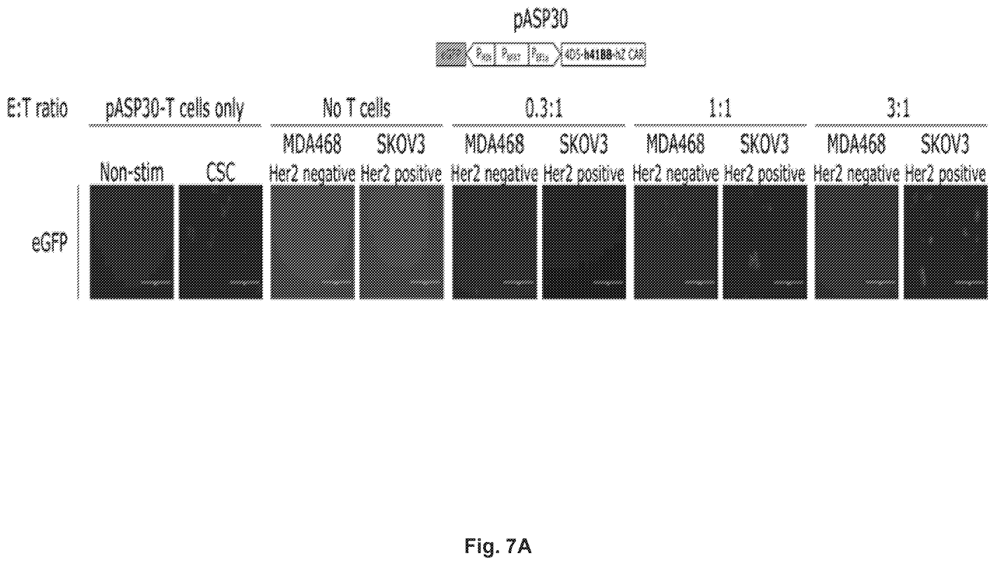

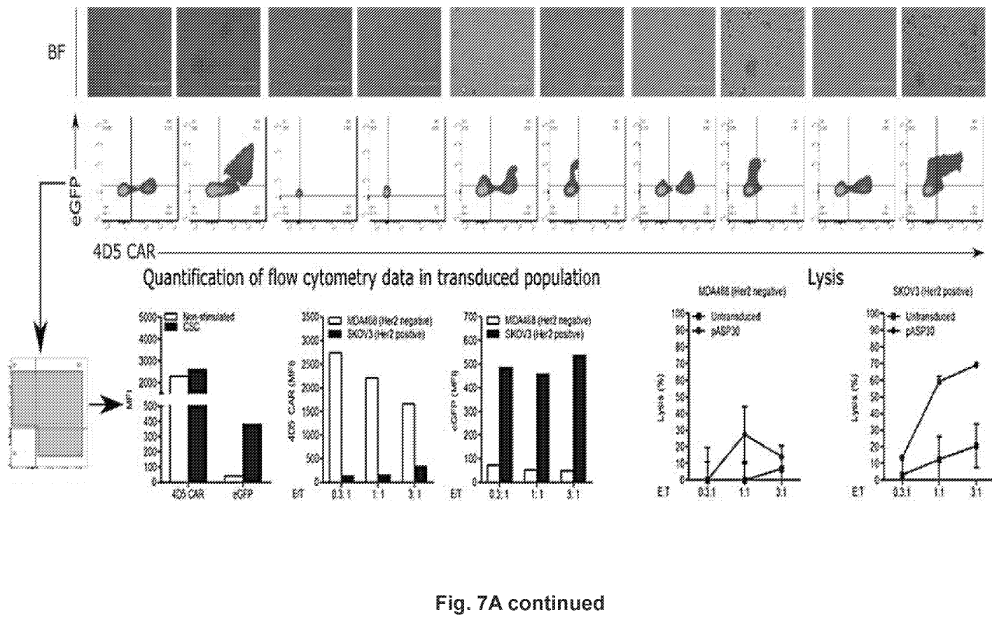

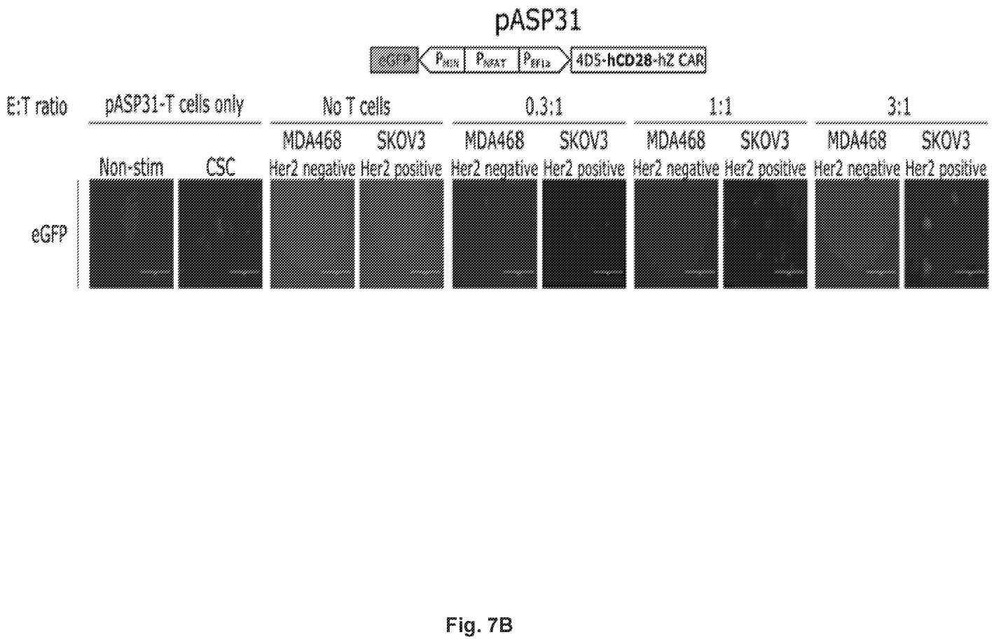

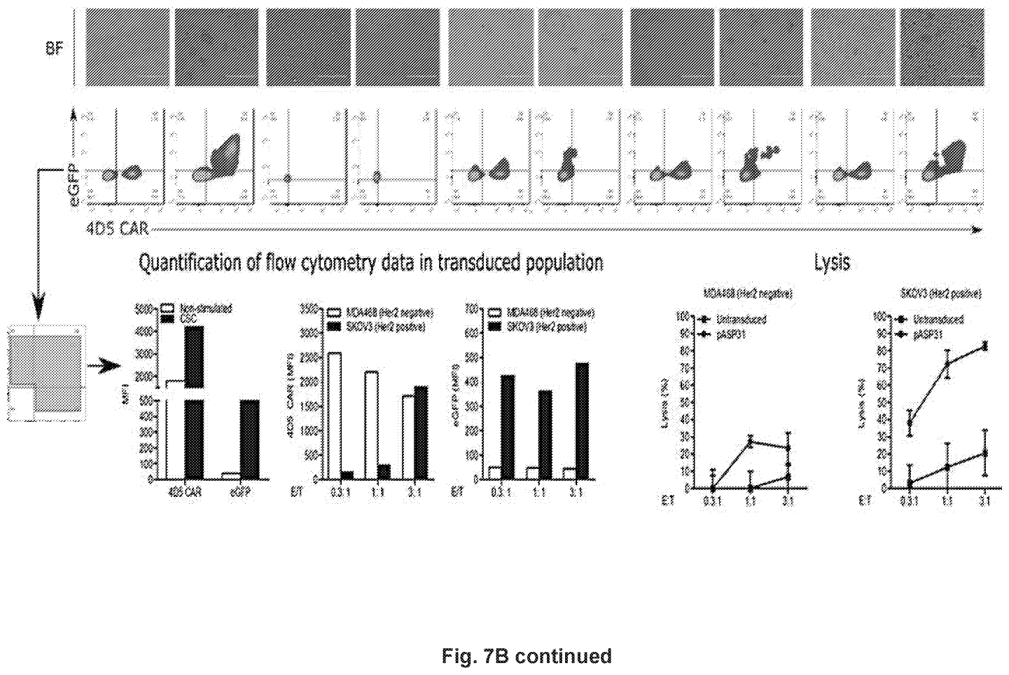

After system optimization, constitutively expressed mCherry was exchanged with CAR to access killing capacity and simultaneous eGFP upregulation to demonstrate a proof of principle. As an example, 4D5 (anti-Her2) scFv in a combination with 4-1BB or CD28 costimulatory domains was used, designated here as pASP30 and pASP31, respectively. Primary T cells were activated, transduced, expanded and rested according to the standard protocol and then co-cultured with SKOV3 cancer cell line that expresses high levels of Her2 at various effector to target (E:T) ratios. As a control MDA468 cell line that does not express significant levels of Her2 was used. Using fluorescence microscopy and flow cytometry, robust increase in eGFP was observed only when pASP30-engineered cells were co-cultured with antigen-positive cell line, whereas the system remained insensitive to the antigen-negative cell lines at all ratios (FIG. 7A). As measured by MFI derived from flow cytometry data (FIG. 7A, bottom left), Her2 positive cell line activated system to the similar levels as CSC which is surprising since CSC shortcuts TCR signaling and hence maximizes T cell activation. Interestingly, surface CAR in engineered T cells dramatically decreased when co-cultured with antigen-positive cell lines, probably because of the known phenomena of CAR internalization (FIG. 7A, flow cytometry and MFI). The higher the E:T ratio, the lower was downregulation of surface CAR, which can be explained by the fraction of CAR molecules engaged with an antigen, which is the highest when there are least T cells in the co-culture. Concomitant with NFAT-regulated eGFP upregulation, pASP30-transduced T cells also specifically killed antigen-positive cancer cells measured by standard luciferase-based assay showing preserved functionalities of T cells when engineered with the developed all-in-one lentiviral vector systems (FIG. 7A, bottom right).

Similar results were observed with pASP31, where the 4-1BB costimulatory domain was exchanged with CD28, meaning that different costimulatory domains did not significantly influence activity of NFAT promoter. Interestingly basal activity of NFAT signaling was also similar suggesting similar tonic activity of CD28 and 4-1BB in the system. However, less downregulation of surface CAR expression and slightly better killing was observed when using CD28 domain compared to 4-1BB, in a co-culture with antigen-positive cell line (FIG. 7B).

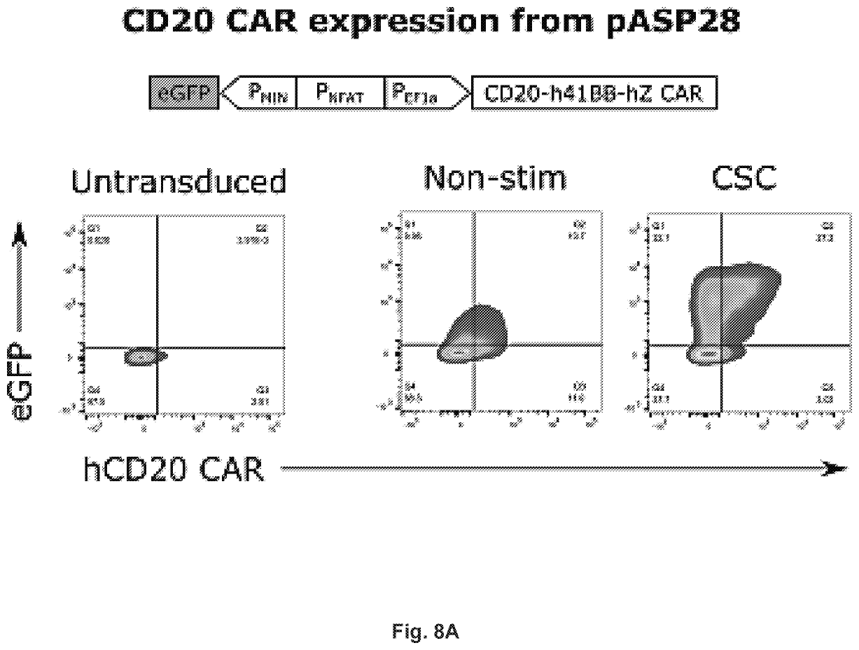

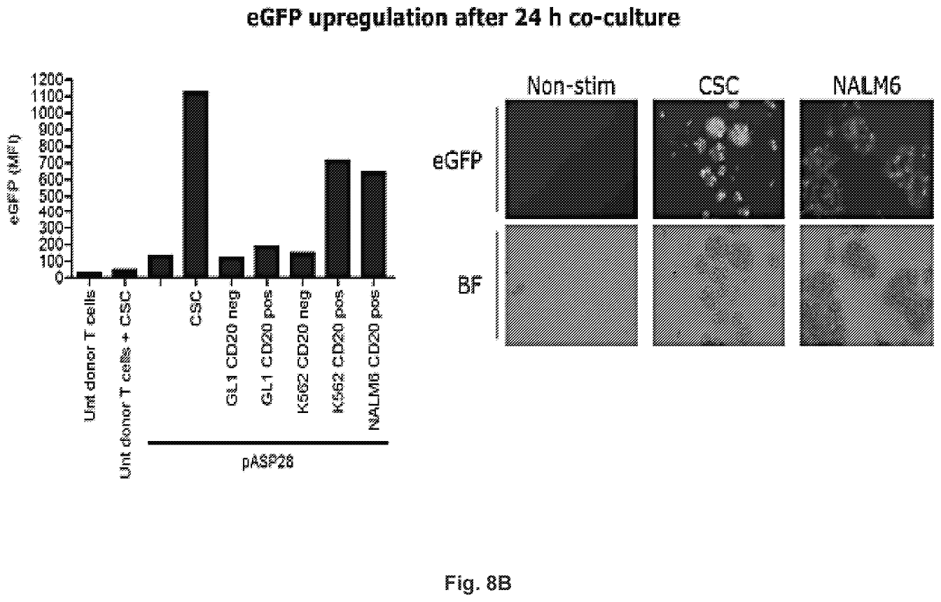

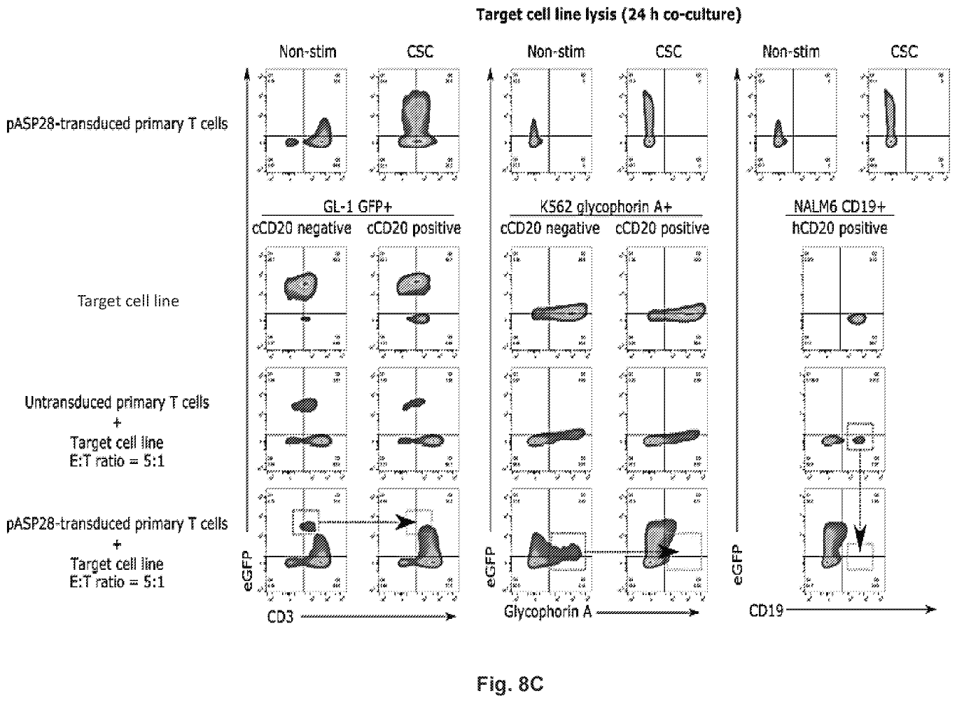

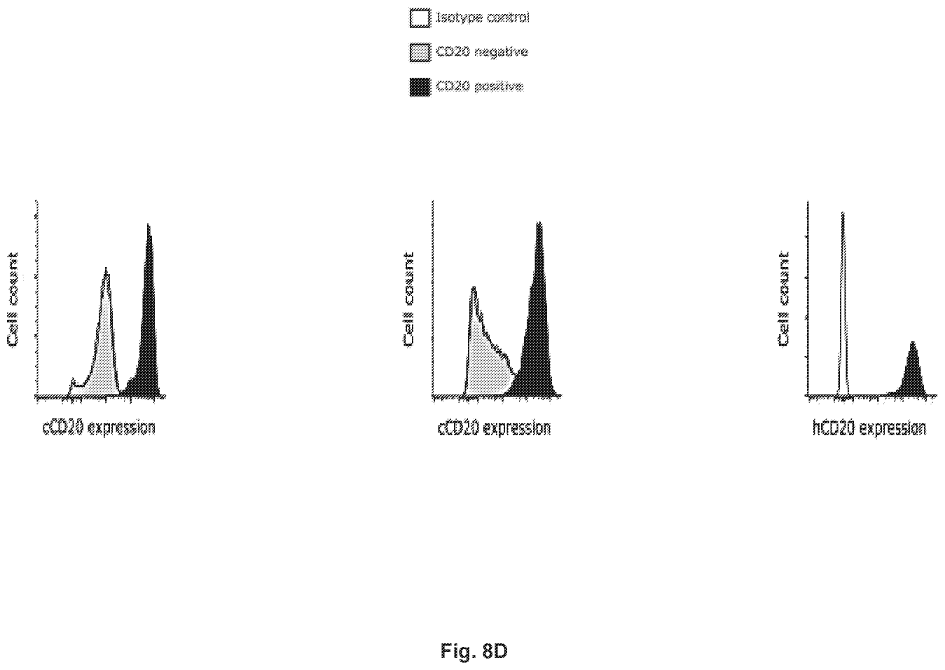

In order to determine functionality of the system in the context of hematological malignancies, Her2 CAR in pASP30 was replaced with anti-CD20 CAR to obtain pASP28 and CAR expression and NFAT-driven eGFP upregulation was verified after CSC stimulation (FIG. 8A). Activated, transduced and rested primary T cells were then co-cultured with cell lines, which endogenously expressed (NALM6) or overexpressed (GL-1 CD20 positive and K562 CD20 positive) CD20 antigen to investigate antigen-dependent response. Robust eGFP expression as measured by MFI was demonstrated when system was stimulated with antigen-positive cell lines K562 and NALM6, reaching 60% of maximal activity achieved by CSC as a positive control (FIG. 8B). Simultaneously with NFAT-driven eGFP upregulation, T cells engineered with pASP28 retained anti-CD20 CAR-mediated lysis capacity as observed by clearance of CD20 positive, but not negative target cell lines determined by flow cytometry. Untransduced T cells did not possess capacity to kill target cell lines (FIG. 8C). Co-culture with CD20 positive GL1 cell line resulted in a very weak expression of inducible part. Without wishing to be bound by theory, different target cell lines may elicit different responses in engineered effector T cells resulting in different kinetics and intensity of NFAT signaling. For example, GL1 cell line is known to be easy-to-lyse by CAR T cells and hence its lysis might be rapid and underlying NFAT-signaling lost quickly. This is important because it demonstrates that activity of the system is operational only when antigen is present and its activity rapidly decreases with distance from antigen, making it local and tumor restricted. Importantly, the system's activity remained at the basal levels in the absence of stimulation signal or when co-cultured with parental antigen-negative cell lines, showing reliable antigen-dependent NFAT-inducible signaling (FIGS. 8A-C).

Taken together, the all-in-one lentiviral system enables antigen-specific lysis and NFAT-driven expression of an inducible molecule when in contact with adherent or suspension cell lines. This enabled the advancement with implementations in the context of solid as well as hematological malignancies.

### Example 7—All-in-One Lentiviral System has Superior Functionalities Compared to Two-Component Lentiviral System

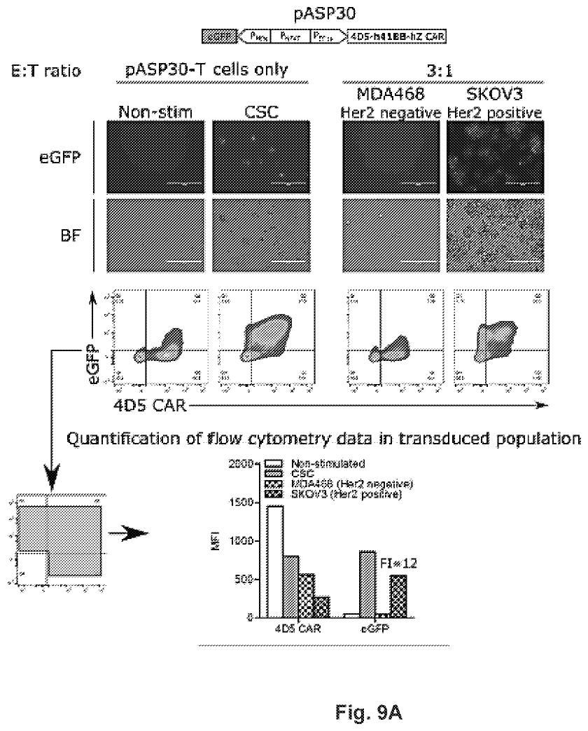

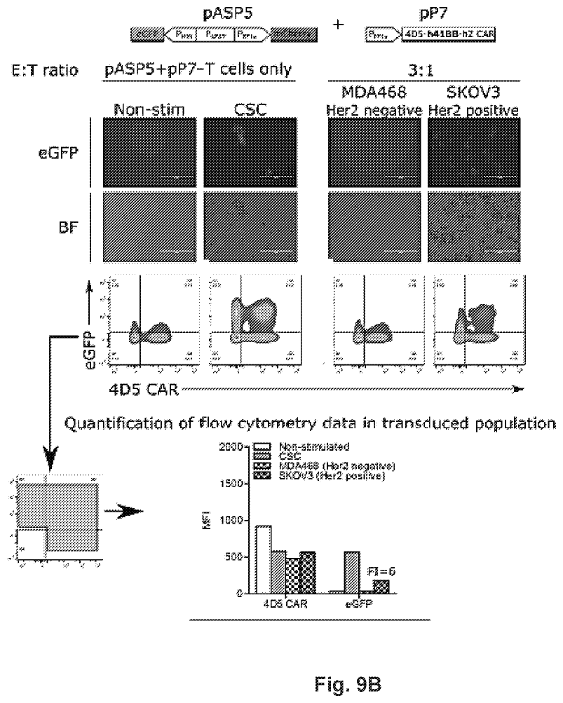

To compare properties and performance of all-in-one with two-component lentiviral systems, the pASP30 was benchmarked, where inducible eGFP and constitutive 4D5 anti-Her2 CAR are encoded in the same lentiviral vector with the system, where the components were broken into two lentiviral vectors, one encoding NFAT-inducible eGFP (pASP5) and other constitutive 4D5 anti-Her2 CAR (pP7). Primary T cells were transcribed with either pASP30 derived virus at MOI3 or a combination of pASP5 and pP7 derived viruses, each added at MOI3. In principle, the number of integrated inducible and constitutive portions should be similar in all-in-one and two-component system; however, the total amount of virus added to the cells was two times higher when two separate viruses were added. In the case of all-in-one lentiviral system, both inducible and constitutive parts were integrated into the same genomic locus at equimolar ratios, since they integrate as a single DNA molecule. To support that, it was demonstrated herein that in the CSC-stimulated primary T cells, the CAR-positive population homogenously shifted up in NFAT-induced eGFP (FIG. 9A). In contrast, it was anticipated that the two-component system would lead to separate integration events of inducible and constitutive parts leading to the non-homogenous distribution, where only a fraction of the cells receives (possibly variable amounts) of both parts. This was demonstrated in FIG. 9B, where three distinct populations (CAR-only, eGFP-only and double positive) were observed after non-specific, CAR-independent stimulation, each at around 20% of the parental population. In contrast, when the cells were activated with target cell lines expressing Her2, one would expect that only CAR-positive cells would express NFAT-inducible eGFP. However, it has already been shown herein that CAR surface expression is downregulated after recognizing cognate ligand, possibly because of internalization, and hence CAR-low and GFP-high populations are observed although they express both the CAR and eGFP (FIGS. 7A-B). Nevertheless, clearly lower expression of eGFP was observed in the CAR-negative portion of the two-component system compared to the CSC regime, which was not the case in all-in-one lentiviral system where every cell that expresses CAR, expresses inducible part as well (FIGS. 9A-B). MFI as a measure of system's capacity showed superior function of all-in-one over the two-component lentiviral system resulting in higher fold induction when stimulated with either non-specific CSC or specific, antigen-driven target cell stimulation (FIGS. 9A-B). Without wishing to be bound by theory, the strong constitutive EF-1alpha promoter may open chromatin and make the NFAT inducible part in the immediate vicinity more accessible for binding of NFAT transcriptional factors and hence may lead to improved performance. Taken together, apart from a simplified modular assembly of lentiviral transfer plasmid and virus production in the all-in-one lentiviral system, where only one virus needs to be produced to achieve constitutive and inducible expression, this system also functionally outperforms two-component system even at lower absolute amount of virus added. Of clinical importance, the all-in-one lentiviral system enables the manufacture of well-defined cell product with homogenous transgene distribution.

FIGS. 14A-14B further illustrate that the single lentiviral system has superior functionalities compared to a two-component lentiviral system. FIGS. 14A-14B show the results of the same experiments as shown in FIGS. 9A-9B, but the data is represented side-by-side, and quantification data is shown in the bar graphs in FIG. 14B.

### Example 8—On Demand Tailored and Reversible Expression of hIL-12 to Increase Efficacy of Engineered Primary T Cells

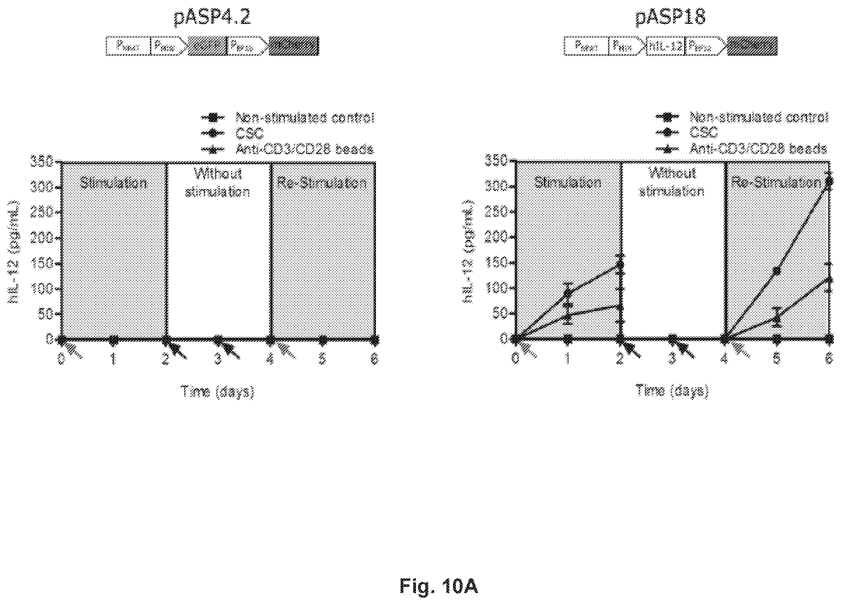

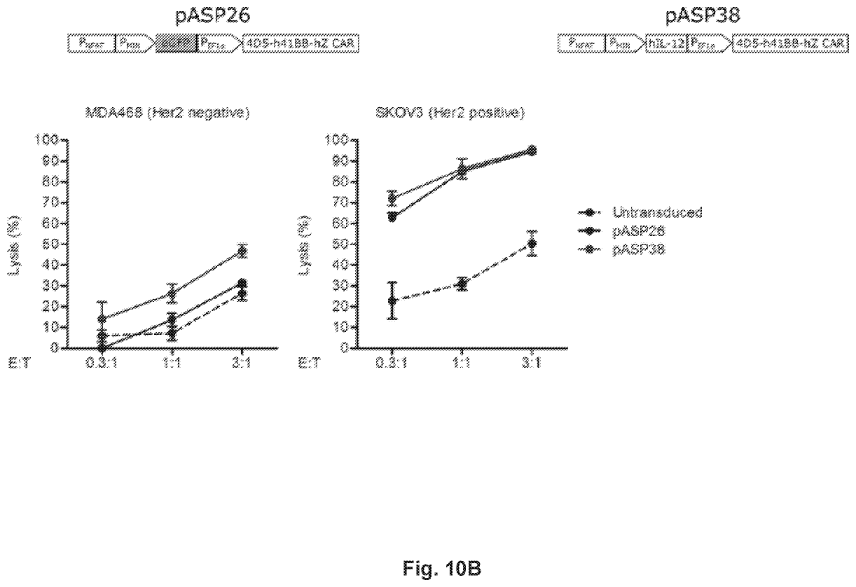

After successful demonstration of constitutive CAR expression that, in a response to a specific antigen, activates the inducible part of the system, the feasibility of expressing an immune modulator to increase efficacy of engineered T cells in an inducible manner was tested. To that end, a pASP18 construct was designed by exchanging eGFP in a pASP4.2 with human IL-12 consisting of p40 and p35 subunits connected with a flexible linker according to the previously published design (Zhang, L. et al. Improving adoptive T cell therapy by targeting and controlling IL-12 expression to the tumor environment. Mol. Ther. 19, 751-9 (2011)). Primary human T cells were activated, transduced with pASP18, expanded and rested according to the standard protocol and then subjected to an activation regime to show expression and reversibility. First, cells were stimulated with CSC or anti-CD3/CD28 beads for 2 days, then washed and left non-stimulated for 2 days and then re-stimulated for another 2 days. Supernatant was collected daily and hIL-12 levels were measured by ELISA. Robust expression was demonstrated when system was stimulated, while there was no detectable IL-12 expression in the absence of stimulation signal, showing reliable performance with a very tight regulation of the effector expression that is needed for safe therapeutic applications. Additionally, the system was fully reversible and as such able to respond to the multiple rounds of antigen encounter (FIG. 10A).

After showing that individual components work as intended, inducible hIL-12 was combined with 4D5 (anti-Her2) scFv CAR with 4-1BB costimulatory domain to obtain construct pASP38. To investigate the effect of inducible hIL-12, a control construct pASP26 was used, where hIL-12 was exchanged by eGFP. Engineered primary T cells were then co-cultured with target cell lines and similar lysis was observed with both constructs after 24 h, demonstrating functionality of engineered cells (FIG. 10B).

When hIL-12 levels were measured in the supernatant of the 24 h co-culture, antigen-dependent and inducible expression of hIL-12 was observed only when co-cultured with Her2 positive SKOV3 cell line but not with Her2 negative MDA468 cell line (FIG. 10C, left). Inducible hIL-12 expression, in turn, increased INF-gamma expression in pASP38-transduced primary T cells compared to pASP26, when co-cultured with target cell lines (FIG. 10C, middle). Importantly, IL-2 remained at similar levels (FIG. 10C, right), demonstrating that inducible hIL-12 specifically shifts engineered cell into the preferable Tₕ1 phenotype, which should in principle increase antitumor efficacy of adoptively transferred CAR/TCR-engineered T cells.

### Example 9—Antigen-Dependent Expression of Engineered Antibody that Blocks IL-6 Signaling to Increase Safety of Engineered Immune Cells

After demonstrating feasibility of increasing efficacy of engineered immune cells, the goal was to advance safety of adoptive cancer immunotherapy. The all-in-one lentiviral system developed in this study enabled the coupling of specific tumor antigen recognition with an inducible expression of an immune modulator to control adverse events associated with immunotherapy such as CRS and neurotoxicity. Currently CRS is most effectively treated nonspecifically with corticosteroids or specifically by administration of Tocilizumab, clinically approved monoclonal antibody that binds interleukin-6 receptor (IL-6R). This blocks interleukin-6 signaling axis, which was shown to be one of the main players in CRS, produced by various immune cells (Neelapu, S. S. et al. Chimeric antigen receptor T-cell therapy—assessment and management of toxicities. Nat. Rev. Clin. Oncol. (2017). doi:10.1038/nrclinonc.2017.148). Physicians apply Tocilizumab only after clinical symptoms appear and often that doe not completely prevent adverse processes. Additionally, therapeutic effect of systemic Tocilizumab administration is limited by blood-brain barrier and would require intra-thecal delivery to control neurological toxicities related to immunotherapy (Johnson, L. A. & June, C. H. Driving gene-engineered T cell immunotherapy of cancer. Cell Res. 27, 38-58 (2016)). Similarly it is expected for other biological drugs that would be used to block various inflammatory mediators in central nervous system.

It was hypothesized that autonomous and early in-situ expression and secretion of interleukin-6 receptor (IL-6R) blocking molecule or other inflammatory blockers (such as neutralizers of tumor-necrosis factor alpha and interleukin beta) by therapeutic immune cells themselves, would prevent CRS-related damage even before it clinically commenced and amplified. Additionally, engineered immune cells that non-intentionally or intentionally (when targeting brain tumors) enter central nervous system would be equipped with build-in features to control accompanying toxicities by local production of inflammatory-blocking molecule, solving the inability of systemically delivered antibodies to cross BBB.

IL-6R blocking antibody was designed by linking Tocilizumab derived heavy and light chains (Tsuchiya, M., Koh, S., Bendig, M. M., Jones, S. T. & Saldanha, J. W. Reshaped human antibody to human interleukin-6 receptor, U.S. Pat. No. 5,817,790) in a scFv format and fused that with human IgG1 Fc to obtain anti-hIL6R scFv-Fc. This designed molecule was cloned as an inducible component combined in reverse back-to-back orientation of the all-in-one lentiviral system with constitutively expressed anti-CD20 CAR to obtain construct pASP52. This construct enabled the targeting of hematological malignancies where CRS and neurological toxicities were demonstrated in clinics.

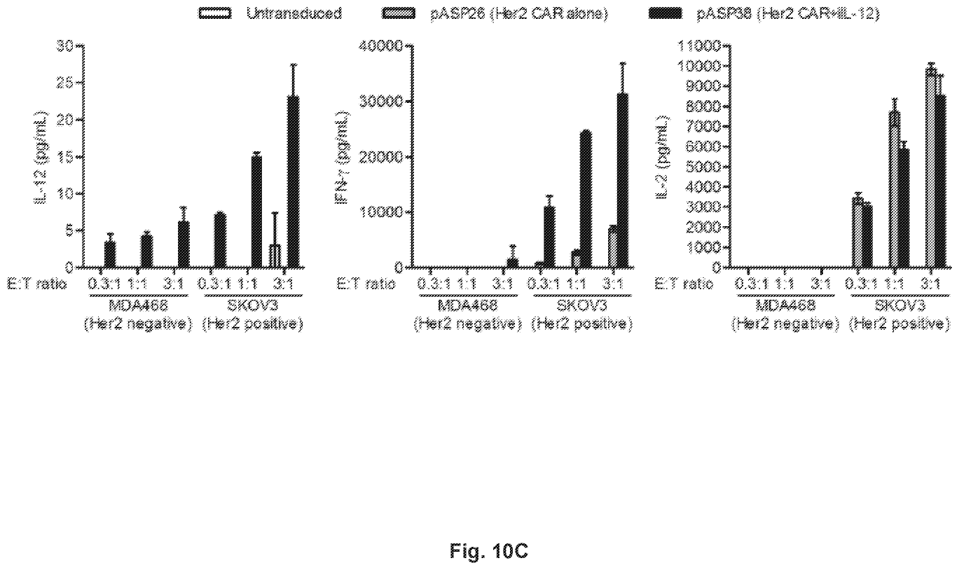

Primary human T cells were activated, transduced with pASP52, expanded and rested according to the standard protocol. Expression of hCD20 CAR was demonstrated and CSC stimulation further increased its level (FIG. 11A), which is in agreement with our previous data showing bi-directional influence in back-to-back orientation that confers increased sensitivity of engineered cells in the direct vicinity of tumor (FIG. 6). Rested effector cells were then co-cultured with cell lines, which endogenously expressed (NALM6) or overexpressed (GL-1 CD20 positive and K562 CD20 positive) CD20 antigen to investigate antigen-dependent response. Supernatants were collected after 72 h and anti-hIL6R scFv-Fc levels were measured by ELISA. Robust expression was demonstrated when system was stimulated with antigen-positive cell lines K562 and NALM6, reaching 60% of maximal activity achieved by CSC as a positive control (FIG. 11B). Activation with CD20 positive GL1 cell line resulted in a lower expression of inducible antibody. Without wishing to be bound by theory, different target cell lines may elicit different responses in engineered effector T cells resulting in different kinetics and intensity of NFAT signaling. For example, GL1 cell line is known to be easy-to-lyse by CAR T cells and hence its lysis might be rapid and underlying NFAT-signaling lost quickly. This is important since it demonstrates that activity of our system occurs only when antigen is present and rapidly decreases making it local and tumor restricted. Importantly, the system's activity remained at the basal levels in the absence of stimulation signal or when co-cultured with parental antigen-negative cell lines, showing reliable antigen-dependent NFAT-inducible signaling (FIG. 11B).

T cells engineered with inducible anti-hIL6R scFv-Fc expression retained anti-CD20 CAR-mediated killing as observed by clearance of CD20 positive, but not negative target cell lines determined by flow cytometry. Untransduced T cells did not possess capacity to kill target cell lines (FIG. 11C). Retaining CAR-mediated killing presents important aspect of our system since it demonstrates that blocking IL-6 signaling axis doesn't prevent cytotoxic activity of engineered cells, while potentially enabling CRS and neurological toxicities management.

An aim of this study was to advance safety and efficacy of adoptive immunotherapy for cancer and to this end engineered immune cells were endowed with a capacity to produce tumor-localized immunomodulatory effector. Specifically, antigen-dependent secretion of engineered anti-hIL6R scFv-Fc was introduced, to address management of CRS and neurological toxicities that accompany successful immunotherapy to address safety aspect. Alternatively, T cells were engineered to express IL-12 to advance efficacy in treating solid tumors. However, since there was no available expression system that could be readily used to engineer T cells with desired features and in clinically relevant and feasible manner, a novel universal all-in-one lentiviral system was developed to meet these tasks.

Provided is an all-in-one lentiviral system for simultaneous constitutive immune receptor and inducible immune modulator expression and its use in a combination with different immune receptors and immune modulators. Two main implementations constitute inducible expression of anti-hIL6R scFv-Fc, a completely novel approach to address safety and IL-12 as an example to address efficacy of adoptive immunotherapy. Supporting data show reliable performance and full functionality according to the design.

Various CARs and TCRs were combined with various immune modulators using the all-in-one lentiviral system provided herein (FIG. 2). To increase efficacy in solid tumors by recruiting endogenous immune responses, increasing persistence, overcoming T cell exhaustion and enabling resistance to checkpoints of implanted T cells, one aspect of the invention combines Her2, mesothelin, folic receptor alpha and EGFRvIII CAR with IL-12, type I interferons, IL-15, IL-18, IL-21, anti-CD25, anti-CD3, anti-CD20, anti-CD40 and anti-PD1 and/or anti-PDL1. Also provided is tumor localized depletion of regulatory T cells using the present system. To increase safety, CD20 and CD19 CARs are combined with anti-inflammatory antibodies that block IL-6, anti-TNFalpha and IL-1 signaling axis to early detect and autonomously suppress cytokine release syndrome and neurological toxicities. To increase penetrance tumor microenvironment restricted expression of tumor stroma degrading enzymes may be tested.

To make a response of the system exclusive to the specific antigen triggered signaling through introduced CAR or TCR, endogenous TCR may be knocked out using a CRISPR/Cas9 system. This may greatly increase safety of engineered T cells over existing NFAT-inducible systems, which will not be able to respond to unpredicted potential antigens through native TCRs.

### Example 10—CRISPR Knock-in

Provided is a vector system and gene construct for dual constitutive and inducible expression in the single (lentiviral) vector. Inducible promoters that function exclusively upon TCR/CAR-signaling are identified. A combination of NFAT responsive element with an optimized minimal promoter P_{MIN} may be used in some embodiments. Various effectors may be used in this configuration, and the system permits knock-in of the effectors under the control of endogenous promoters that are induced in activated immune cells after specific antigen recognition. Also provided is a CRISPR-related system-mediated knock-in of the designed expression system into the TCR or HLA (or other relevant loci) to make introduced receptor-mediated signaling the exclusive activator of the system (and simultaneously prevent TCR-mediated graft-versus-host disease) and to make engineered cells universal, respectively. In some embodiments, the designed expression cassette is encoded in a donor template. In some embodiments, the donor template is encoded in a viral vector. In some embodiments the viral vector is an adeno-associated viral vector. In further embodiments, the adeno-associated viral vector is AAV6. In some embodiments, the CRISPR-related system is a CRISPR/Cas9 system.

### Example 11—Effect on Engineered T Cells of Blocking IL-6 Signaling

Construct pASP52 is compared with the same construct where anti-hIL-6R scFv-Fc is replaced by eGFP to benchmark and analyze the effect of interfering with IL-6 signaling axis on the functionality of engineered T cells in vitro and in vivo.

The biological activity and capacity of a designed effector to block IL-6 mediated signaling is also determined.

### Example 12—Further Constructs

Constructs as described in Example 8 are generated and tested, where IL-12 effector may be replaced with IL-18, type I interferons, and other immunomodulatory molecules which are disclosed herein for use in the system.

Nucleotide sequences-all are listed in 5′-3′
direction
Building elements
1. Synthetic NFAT promoter (6 x consensus NFAT
binding sites)
(SEQ ID NO: 5)
GGAGGAAAAACTGTTTCATACAGAAGGCGTGGAGGAAAAACTGTTTCA
TACAGAAGGCGTGGAGGAAAAACTGTTTCATACAGAAGGCGTGGAGGA
AAAACTGTTTCATACAGAAGGCGTGGAGGAAAAACTGTTTCATACAGA
AGGCGTGGAGGAAAAACTGTTTCATACAGAAGGCGT
2. Minimal promoter
(SEQ ID NO: 6)
TAGAGGGTATATAATGGAAGCTCGACTTCCAG
3. EF1α promoter
(SEQ ID NO: 7)
CGTGAGGCTCCGGTGCCCGTCAGTGGGCAGAGCGCACATCGCCCACAG
TCCCCGAGAAGTTGGGGGGAGGGGTCGGCAATTGAACCGGTGCCTAGA
GAAGGTGGCGCGGGGTAAACTGGGAAAGTGATGTCGTGTACTGGCTCC
GCCTTTTTCCCGAGGGTGGGGGAGAACCGTATATAAGTGCAGTAGTCG
CCGTGAACGTTCTTTTTCGCAACGGGTTTGCCGCCAGAACACAGGTAA
GTGCCGTGTGTGGTTCCCGCGGGCCTGGCCTCTTTACGGGTTATGGCC
CTTGCGTGCCTTGAATTACTTCCACCTGGCTGCAGTACGTGATTCTTG
ATCCCGAGCTTCGGGTTGGAAGTGGGTGGGAGAGTTCGAGGCCTTGCG
CTTAAGGAGCCCCTTCGCCTCGTGCTTGAGTTGAGGCCTGGCCTGGGC
GCTGGGGCCGCCGCGTGCGAATCTGGTGGCACCTTCGCGCCTGTCTCG
CTGCTTTCGATAAGTCTCTAGCCATTTAAAATTTTTGATGACCTGCTG
CGACGCTTTTTTTCTGGCAAGATAGTCTTGTAAATGCGGGCCAAGATC
TGCACACTGGTATTTCGGTTTTTGGGGCCGCGGGCGGCGACGGGGCCC
GTGCGTCCCAGCGCACATGTTCGGCGAGGCGGGGCCTGCGAGCGCGGC
CACCGAGAATCGGACGGGGGTAGTCTCAAGCTGGCCGGCCTGCTCTGG
TGCCTGGCCTCGCGCCGCCGTGTATCGCCCCGCCCTGGGCGGCAAGGC
TGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGC
GGGCGGGTGAGTCACCCACACAAAGGAAAAGGGCCTTTCCGTCCTCAG
CCGTCGCTTCATGTGACTCCACTGAGTACCGGGCGCCGTCCAGGCACC
TCGATTAGTTCTCGTGCTTTTGGAGTACGTCGTCTTTAGGTTGGGGGG
AGGGGTTTTATGCGATGGAGTTTCCCCACACTGAGTGGGTGGAGACTG
AAGTTAGGCCAGCTTGGCACTTGATGTAATTCTCCTTGGAATTTGCCC
TTTTTGAGTTTGGATCTTGGTTCATTCTCAAGCCTCAGACAGTGGTTC
AAAGTTTTTTTCTTCCATTTCAGGTGTCGTGA
4. Spacer + Kozak sequence (this is the sequence
between 3′ end of the minimal promoter
or any inducible promoter and ATG start codon of
constitutively expressed module and
enables functional spacing between transcription
start site and translational start site)
(SEQ ID NO: 8)
GGCATTCCGGTACTGTTGGTAAAGCCACC
5. Example of a functionally connected
regulatory unit for an inducible module followed
by constitutive module in the same direction.
Same configuration is used when inducible and
constitutive modules are placed back to back in
the opposite direction, where inducible
module is placed in reverse and constitutive
module in forward orientation.
(SEQ ID NO: 9)
GGAGGAAAAACTGTTTCATACAGAAGGCGTGGAGGAAAAACTGTTTCA
TACAGAAGGCGTGGAGGAAAAACTGTTTCATACAGAAGGCGTGGAGGA
AAAACTGTTTCATACAGAAGGCGTGGAGGAAAAACTGTTTCATACAGA

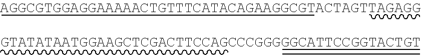

TGGTAAAGCCACC-
followed by immune modulator starting with a
start codon and ending with stop codon-followed
by strong constitutive promoter such as EF1α-
followed by immune receptor such as
CAR or TCR starting with start codon and ending
with stop codon.
Synthetic NFAT promoter: underline
Minimal promoter: wavy underline
Spacer: double underline

### Constructs Used for Experiments:

1. pASP4.2 (orientation of inducible and constitutive modules: same direction)
(SEQ ID NO: 10)
TCGAGTTAATTAAGCTAGCCCGGTACTGTTGGAGGAAAAACTGTTTCATACAGAAGGCGTGGAGGAAAAACTGTTTCATACAGAAGGC
GTGGAGGAAAAACTGTTTCATACAGAAGGCGTGGAGGAAAAACTGTTTCATACAGAAGGCGTGGAGGAAAAACTGTTTCATACAGAAG
GCGTGGAGGAAAAACTGTTTCATACAGAAGGCGTACTAGTTAGAGGGTATATAATGGAAGCTCGACTTCCAGCCCGGGGGCATTCCGG
TACTGTTGGTAAAGCCACCATGGTGAGCAAGGGCGAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGACGACGGCAACTACAAGACCCGCGCC
GAGGTGAAGTTCGAGGGCGACACCCTGGTGAACCGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGACGGCCCCGTGCTGCTGCCCGACAAC
CACTACCTGAGCACCCAGTCCGCCCTGAGCAAAGACCCCAACGAGAAGCGCGATCACATGGTCCTGCTGGAGTTCGTGACCGCCGCCG
GGATCACTCTCGGCATGGACGAGCTGTACAAGTAAATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
GCCACCATGGTGAGCAAGGGCGAGGAGGATAACATGGCCATCATCAAGGAGTTCATGCGCTTCAAGGTGCACATGGAGGGCTCCGTGA
ACGGCCACGAGTTCGAGATCGAGGGCGAGGGCGAGGGCCGCCCCTACGAGGGCACCCAGACCGCCAAGCTGAAGGTGACCAAGGGTGG
CCCCCTGCCCTTCGCCTGGGACATCCTGTCCCCTCAGTTCATGTACGGCTCCAAGGCCTACGTGAAGCACCCCGCCGACATCCCCGAC
TACTTGAAGCTGTCCTTCCCCGAGGGCTTCAAGTGGGAGCGCGTGATGAACTTCGAGGACGGCGGCGTGGTGACCGTGACCCAGGACT
CCTCCCTGCAGGACGGCGAGTTCATCTACAAGGTGAAGCTGCGCGGCACCAACTTCCCCTCCGACGGCCCCGTAATGCAGAAGAAGAC
CATGGGCTGGGAGGCCTCCTCCGAGCGGATGTACCCCGAGGACGGCGCCCTGAAGGGCGAGATCAAGCAGAGGCTGAAGCTGAAGGAC
GGCGGCCACTACGACGCTGAGGTCAAGACCACCTACAAGGCCAAGAAGCCCGTGCAGCTGCCCGGCGCCTACAACGTCAACATCAAGT
TGGACATCACCTCCCACAACGAGGACTACACCATCGTGGAACAGTACGAACGCGCCGAGGGCCGCCACTCCACCGGCGGCATGGACGA
GCTGTACAAGAGGGCCAAGAGGCGTACGTAAG
2. pASP5 (orientation of inducible and constitutive modules: opposite direction)
(SEQ ID NO: 11)
TCGAGTTAATTAAGCTAGCTTACTTGTACAGCTCGTCCATGCCGAGAGTGATCCCGGCGGCGGTCACGAACTCCAGCAGGACCATGTG
ATCGCGCTTCTCGTTGGGGTCTTTGCTCAGGGCGGACTGGGTGCTCAGGTAGTGGTTGTCGGGCAGCAGCACGGGGCCGTCGCCGATG
GGGGTGTTCTGCTGGTAGTGGTCGGCGAGCTGCACGCTGCCGTCCTCGATGTTGTGGCGGATCTTGAAGTTCACCTTGATGCCGTTCT
TCTGCTTGTCGGCCATGATATAGACGTTGTGGCTGTTGTAGTTGTACTCCAGCTTGTGCCCCAGGATGTTGCCGTCCTCCTTGAAGTC
GATGCCCTTCAGCTCGATGCGGTTCACCAGGGTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTGCTGCTTCATGTGGTCGGGGTAGCGGCTGAAGC
ACTGCACGCCGTAGGTCAGGGTGGTCACGAGGGTGGGCCAGGGCACGGGCAGCTTGCCGGTGGTGCAGATGAACTTCAGGGTCAGCTT
GCCGTAGGTGGCATCGCCCTCGCCCTCGCCGGACACGCTGAACTTGTGGCCGTTTACGTCGCCGTCCAGCTCGACCAGGATGGGCACC
ACCCCGGTGAACAGCTCCTCGCCCTTGCTCACCATGGTGGCTTTACCAACAGTACCGGAATGCCCCCGGGCTGGAAGTCGAGCTTCCA
TTATATACCCTCTAACTAGTACGCCTTCTGTATGAAACAGTTTTTCCTCCACGCCTTCTGTATGAAACAGTTTTTCCTCCACGCCTTC
TGTATGAAACAGTTTTTCCTCCACGCCTTCTGTATGAAACAGTTTTTCCTCCACGCCTTCTGTATGAAACAGTTTTTCCTCCACGCCT
TCTGTATGAAACAGTTTTTCCTCCAACAGTACCGGATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
GCCACCATGGTGAGCAAGGGCGAGGAGGATAACATGGCCATCATCAAGGAGTTCATGCGCTTCAAGGTGCACATGGAGGGCTCCGTGA
ACGGCCACGAGTTCGAGATCGAGGGCGAGGGCGAGGGCCGCCCCTACGAGGGCACCCAGACCGCCAAGCTGAAGGTGACCAAGGGTGG
CCCCCTGCCCTTCGCCTGGGACATCCTGTCCCCTCAGTTCATGTACGGCTCCAAGGCCTACGTGAAGCACCCCGCCGACATCCCCGAC
TACTTGAAGCTGTCCTTCCCCGAGGGCTTCAAGTGGGAGCGCGTGATGAACTTCGAGGACGGCGGCGTGGTGACCGTGACCCAGGACT
CCTCCCTGCAGGACGGCGAGTTCATCTACAAGGTGAAGCTGCGCGGCACCAACTTCCCCTCCGACGGCCCCGTAATGCAGAAGAAGAC
CATGGGCTGGGAGGCCTCCTCCGAGCGGATGTACCCCGAGGACGGCGCCCTGAAGGGCGAGATCAAGCAGAGGCTGAAGCTGAAGGAC
GGCGGCCACTACGACGCTGAGGTCAAGACCACCTACAAGGCCAAGAAGCCCGTGCAGCTGCCCGGCGCCTACAACGTCAACATCAAGT
TGGACATCACCTCCCACAACGAGGACTACACCATCGTGGAACAGTACGAACGCGCCGAGGGCCGCCACTCCACCGGCGGCATGGACGA
GCTGTACAAGAGGGCCAAGAGGCGTACGTAAG
3. pASP7 (orientation of inducible and constitutive modules: opposite direction)
(SEQ ID NO: 12)
TCGAGTACCACATTTGTAGAGGTTTTACTTGCTTTAAAAAACCTCCCACACCTCCCCCTGAACCTGAAACATAAAATGAATGCAATTG
TTGTTGTTAACTTGTTTATTGCAGCTTATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTTAATTAAGCTAGCTTACTTGTACAGCTCGTCCATGC
CGAGAGTGATCCCGGCGGCGGTCACGAACTCCAGCAGGACCATGTGATCGCGCTTCTCGTTGGGGTCTTTGCTCAGGGCGGACTGGGT
GCTCAGGTAGTGGTTGTCGGGCAGCAGCACGGGGCCGTCGCCGATGGGGGTGTTCTGCTGGTAGTGGTCGGCGAGCTGCACGCTGCCG
TCCTCGATGTTGTGGCGGATCTTGAAGTTCACCTTGATGCCGTTCTTCTGCTTGTCGGCCATGATATAGACGTTGTGGCTGTTGTAGT
TGTACTCCAGCTTGTGCCCCAGGATGTTGCCGTCCTCCTTGAAGTCGATGCCCTTCAGCTCGATGCGGTTCACCAGGGTGTCGCCCTC
GAACTTCACCTCGGCGCGGGTCTTGTAGTTGCCGTCGTCCTTGAAGAAGATGGTGCGCTCCTGGACGTAGCCTTCGGGCATGGCGGAC
TTGAAGAAGTCGTGCTGCTTCATGTGGTCGGGGTAGCGGCTGAAGCACTGCACGCCGTAGGTCAGGGTGGTCACGAGGGTGGGCCAGG
GCACGGGCAGCTTGCCGGTGGTGCAGATGAACTTCAGGGTCAGCTTGCCGTAGGTGGCATCGCCCTCGCCCTCGCCGGACACGCTGAA
CTTGTGGCCGTTTACGTCGCCGTCCAGCTCGACCAGGATGGGCACCACCCCGGTGAACAGCTCCTCGCCCTTGCTCACCATGGTGGCT
TTACCAACAGTACCGGAATGCCCCCGGGCTGGAAGTCGAGCTTCCATTATATACCCTCTAACTAGTACGCCTTCTGTATGAAACAGTT
TTTCCTCCACGCCTTCTGTATGAAACAGTTTTTCCTCCACGCCTTCTGTATGAAACAGTTTTTCCTCCACGCCTTCTGTATGAAACAG
TTTTTCCTCCACGCCTTCTGTATGAAACAGTTTTTCCTCCACGCCTTCTGTATGAAACAGTTTTTCCTCCAACAGTACCGGATGCATC
GTGAGGCTCCGGTGCCCGTCAGTGGGCAGAGCGCACATCGCCCACAGTCCCCGAGAAGTTGGGGGGAGGGGTCGGCAATTGAACCGGT
GCCTAGAGAAGGTGGCGCGGGGTAAACTGGGAAAGTGATGTCGTGTACTGGCTCCGCCTTTTTCCCGAGGGTGGGGGAGAACCGTATA
TAAGTGCAGTAGTCGCCGTGAACGTTCTTTTTCGCAACGGGTTTGCCGCCAGAACACAGGTAAGTGCCGTGTGTGGTTCCCGCGGGCC
TGGCCTCTTTACGGGTTATGGCCCTTGCGTGCCTTGAATTACTTCCACCTGGCTGCAGTACGTGATTCTTGATCCCGAGCTTCGGGTT
GGAAGTGGGTGGGAGAGTTCGAGGCCTTGCGCTTAAGGAGCCCCTTCGCCTCGTGCTTGAGTTGAGGCCTGGCCTGGGCGCTGGGGCC
GCCGCGTGCGAATCTGGTGGCACCTTCGCGCCTGTCTCGCTGCTTTCGATAAGTCTCTAGCCATTTAAAATTTTTGATGACCTGCTGC
GACGCTTTTTTTCTGGCAAGATAGTCTTGTAAATGCGGGCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGCGCGGCCACCGAGAATCGGACGGGGGTAGTCTCAAGCTG
GCCGGCCTGCTCTGGTGCCTGGCCTCGCGCCGCCGTGTATCGCCCCGCCCTGGGCGGCAAGGCTGGCCCGGTCGGCACCAGTTGCGTG
AGCGGAAAGATGGCCGCTTCCCGGCCCTGCTGCAGGGAGCTCAAAATGGAGGACGCGGCGCTCGGGAGAGCGGGCGGGTGAGTCACCC
ACACAAAGGAAAAGGGCCTTTCCGTCCTCAGCCGTCGCTTCATGTGACTCCACTGAGTACCGGGCGCCGTCCAGGCACCTCGATTAGT
TCTCGTGCTTTTGGAGTACGTCGTCTTTAGGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTGGATCTTGGTTCATTCTCAAGCCTCAGACA
GTGGTTCAAAGTTTTTTTCTTCCATTTCAGGTGTCGTGATTCTAGAGCCACCATGGTGAGCAAGGGCGAGGAGGATAACATGGCCATC
ATCAAGGAGTTCATGCGCTTCAAGGTGCACATGGAGGGCTCCGTGAACGGCCACGAGTTCGAGATCGAGGGCGAGGGCGAGGGCCGCC
CCTACGAGGGCACCCAGACCGCCAAGCTGAAGGTGACCAAGGGTGGCCCCCTGCCCTTCGCCTGGGACATCCTGTCCCCTCAGTTCAT
GTACGGCTCCAAGGCCTACGTGAAGCACCCCGCCGACATCCCCGACTACTTGAAGCTGTCCTTCCCCGAGGGCTTCAAGTGGGAGCGC
GTGATGAACTTCGAGGACGGCGGCGTGGTGACCGTGACCCAGGACTCCTCCCTGCAGGACGGCGAGTTCATCTACAAGGTGAAGCTGC
GCGGCACCAACTTCCCCTCCGACGGCCCCGTAATGCAGAAGAAGACCATGGGCTGGGAGGCCTCCTCCGAGCGGATGTACCCCGAGGA
CGGCGCCCTGAAGGGCGAGATCAAGCAGAGGCTGAAGCTGAAGGACGGCGGCCACTACGACGCTGAGGTCAAGACCACCTACAAGGCC
AAGAAGCCCGTGCAGCTGCCCGGCGCCTACAACGTCAACATCAAGTTGGACATCACCTCCCACAACGAGGACTACACCATCGTGGAAC
AGTACGAACGCGCCGAGGGCCGCCACTCCACCGGCGGCATGGACGAGCTGTACAAGAGGGCCAAGAGGCGTACGTAAG
4. pASP8 (orientation of inducible and constitutive modules: same direction)
(SEQ ID NO: 13)
TCGAGTTAATTAAGCTAGCAAGGCTTTCTGTTTCCTGCATTCTTACTTCTTACTACGTGATACATCTAGTCACCAGGGAAGAAGCGAA
TGACACACTTCCAAAAACCAATTCGTAGCTTTCTAAATAAAACCCTTTCTAGCTGGAGAGAGATCCATGAGCATAGAGATCTTAAAAT
TCATGTTCAGCAATAAATCCTGGGGCCCCAGACAGTGTCAGGTGCATAGGGGGTGTTCAGTAAATATCAGTTAAATGTTGCATAAATC
CGATAAACGGGATTCCTGGAAAATACTACACTCTCCTTCTCCAAATTATCTTCATCTCAAAGACAGGAACCTCTAACTTTTAATTCTT
TACTTAGATTATGCTGTCTCCTAAACTGTTTATGTTTTCTGAAATTTAAGGCAGGATGTCTCAGAGTCTGGGAAAATCCCACTTTCCT
CCTGCTACACCTTACAGTTGTGAGAAAGCACATTTCAGACAACAGGGAAAACCCATACTTCACCACAACAACACACTATACATTGTCT
GGTCCACTGGAGCATAAATTAAAGAGAAACAATGTAGTCAAGCAAGTAGGCGGCAAGAGGAAGGGGGCGGAGACATCATCAGGGAGTA
TAAACTCTGAGATGCCTCAGAGCCTCACAGACTCAACAAGAGCTCCAGCAAAGACTTTCACTGTAGCTTGACTTGACCTGAGATTAAC
TAGGGAATCTTGAGAATAAAGCCCGGGGGCATTCCGGTACTGTTGGTAAAGCCACCATGGTGAGCAAGGGCGAGGAGCTGTTCACCGG
GGTGGTGCCCATCCTGGTCGAGCTGGACGGCGACGTAAACGGCCACAAGTTCAGCGTGTCCGGCGAGGGCGAGGGCGATGCCACCTAC
GGCAAGCTGACCCTGAAGTTCATCTGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTGAGCAAAGACCCCAACGAGAAGCGC
GATCACATGGTCCTGCTGGAGTTCGTGACCGCCGCCGGGATCACTCTCGGCATGGACGAGCTGTACAAGTAAATGCATCGTGAGGCTC
CGGTGCCCGTCAGTGGGCAGAGCGCACATCGCCCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTAAGTGCCGTGTGTGGTTCCCGCGGGCCTGGCCTCTT
TACGGGTTATGGCCCTTGCGTGCCTTGAATTACTTCCACCTGGCTGCAGTACGTGATTCTTGATCCCGAGCTTCGGGTTGGAAGTGGG
TGGGAGAGTTCGAGGCCTTGCGCTTAAGGAGCCCCTTCGCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTAGCCATTTAAAATTTTTGATGACCTGCTGCGACGCTTTT
TTTCTGGCAAGATAGTCTTGTAAATGCGGGCCAAGATCTGCACACTGGTATTTCGGTTTTTGGGGCCGCGGGCGGCGACGGGGCCCGT
GCGTCCCAGCGCACATGTTCGGCGAGGCGGGGCCTGCGAGCGCGGCCACCGAGAATCGGACGGGGGTAGTCTCAAGCTGGCCGGCCTG
CTCTGGTGCCTGGCCTCGCGCCGCCGTGTATCGCCCCGCCCTGGGCGGCAAGGCTGGCCCGGTCGGCACCAGTTGCGTGAGCGGAAAG
ATGGCCGCTTCCCGGCCCTGCTGCAGGGAGCTCAAAATGGAGGACGCGGCGCTCGGGAGAGCGGGCGGGTGAGTCACCCACACAAAGG
AAAAGGGCCTTTCCGTCCTCAGCCGTCGCTTCATGTGACTCCACTGAGTACCGGGCGCCGTCCAGGCACCTCGATTAGTTCTCGTGCT
TTTGGAGTACGTCGTCTTTAGGTTGGGGGGAGGGGTTTTATGCGATGGAGTTTCCCCACACTGAGTGGGTGGAGACTGAAGTTAGGCC
AGCTTGGCACTTGATGTAATTCTCCTTGGAATTTGCCCTTTTTGAGTTTGGATCTTGGTTCATTCTCAAGCCTCAGACAGTGGTTCAA
AGTTTTTTTCTTCCATTTCAGGTGTCGTGATTCTAGAGCCACCATGGTGAGCAAGGGCGAGGAGGATAACATGGCCATCATCAAGGAG
TTCATGCGCTTCAAGGTGCACATGGAGGGCTCCGTGAACGGCCACGAGTTCGAGATCGAGGGCGAGGGCGAGGGCCGCCCCTACGAGG
GCACCCAGACCGCCAAGCTGAAGGTGACCAAGGGTGGCCCCCTGCCCTTCGCCTGGGACATCCTGTCCCCTCAGTTCATGTACGGCTC
CAAGGCCTACGTGAAGCACCCCGCCGACATCCCCGACTACTTGAAGCTGTCCTTCCCCGAGGGCTTCAAGTGGGAGCGCGTGATGAAC
TTCGAGGACGGCGGCGTGGTGACCGTGACCCAGGACTCCTCCCTGCAGGACGGCGAGTTCATCTACAAGGTGAAGCTGCGCGGCACCA
ACTTCCCCTCCGACGGCCCCGTAATGCAGAAGAAGACCATGGGCTGGGAGGCCTCCTCCGAGCGGATGTACCCCGAGGACGGCGCCCT
GAAGGGCGAGATCAAGCAGAGGCTGAAGCTGAAGGACGGCGGCCACTACGACGCTGAGGTCAAGACCACCTACAAGGCCAAGAAGCCC
GTGCAGCTGCCCGGCGCCTACAACGTCAACATCAAGTTGGACATCACCTCCCACAACGAGGACTACACCATCGTGGAACAGTACGAAC
GCGCCGAGGGCCGCCACTCCACCGGCGGCATGGACGAGCTGTACAAGAGGGCCAAGAGGCGTACGTAAG
5. pASP9 (orientation of inducible and constitutive modules: same direction)
(SEQ ID NO: 14)
TCGAGTTAATTAAGCTAGCAATGCTTTGTCCTGGAGAGCTATCTTAAGGGACAAAATCGTTTTCCCAGCGTCATCTGTGACACATCCT
GACAGTAGAGAGCTGCTTCCAAGAAGCAATTTGAAGTGCCATTATCAGGCAGGGACGGGGGCTCTAGGGGATTTCGGGGTCAGCAGAT
ATGAAATGAATGATTTCATAGGGCTGTCACAGAGCTGTGGTGGGAATTTCCCATGAGACCCCGCCCCTGGCTGAGTCACCGCACTCCT
GTGTTTGACCTGAAGTCCTCTCGTGCTGCAGAAGCCTGAAGACCAAGGAGTGGAAAGTTCTCCGGCAGCCCTGAGATCTCCCGGGGGC
ATTCCGGTACTGTTGGTAAAGCCACCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGCAAGCTGACCCTGAAGTTCATCTGCACC
ACCGGCAAGCTGCCCGTGCCCTGGCCCACCCTCGTGACCACCCTGACCTACGGCGTGCAGTGCTTCAGCCGCTACCCCGACCACATGA
AGCAGCACGACTTCTTCAAGTCCGCCATGCCCGAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGGGCATCGACTTCAAGGAGGACGGCAACATCCTG
GGGCACAAGCTGGAGTACAACTACAACAGCCACAACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAGAACACCCCCATCGGCGACGGCCCCGTGCTGCTGCC
CGACAACCACTACCTGAGCACCCAGTCCGCCCTGAGCAAAGACCCCAACGAGAAGCGCGATCACATGGTCCTGCTGGAGTTCGTGACC
GCCGCCGGGATCACTCTCGGCATGGACGAGCTGTACAAGTAAATGCATCGTGAGGCTCCGGTGCCCGTCAGTGGGCAGAGCGCACATC
GCCCACAGTCCCCGAGAAGTTGGGGGGAGGGGTCGGCAATTGAACCGGTGCCTAGAGAAGGTGGCGCGGGGTAAACTGGGAAAGTGAT
GTCGTGTACTGGCTCCGCCTTTTTCCCGAGGGTGGGGGAGAACCGTATATAAGTGCAGTAGTCGCCGTGAACGTTCTTTTTCGCAACG
GGTTTGCCGCCAGAACACAGGTAAGTGCCGTGTGTGGTTCCCGCGGGCCTGGCCTCTTTACGGGTTATGGCCCTTGCGTGCCTTGAAT
TACTTCCACCTGGCTGCAGTACGTGATTCTTGATCCCGAGCTTCGGGTTGGAAGTGGGTGGGAGAGTTCGAGGCCTTGCGCTTAAGGA
GCCCCTTCGCCTCGTGCTTGAGTTGAGGCCTGGCCTGGGCGCTGGGGCCGCCGCGTGCGAATCTGGTGGCACCTTCGCGCCTGTCTCG
CTGCTTTCGATAAGTCTCTAGCCATTTAAAATTTTTGATGACCTGCTGCGACGCTTTTTTTCTGGCAAGATAGTCTTGTAAATGCGGG
CCAAGATCTGCACACTGGTATTTCGGTTTTTGGGGCCGCGGGCGGCGACGGGGCCCGTGCGTCCCAGCGCACATGTTCGGCGAGGCGG
GGCCTGCGAGCGCGGCCACCGAGAATCGGACGGGGGTAGTCTCAAGCTGGCCGGCCTGCTCTGGTGCCTGGCCTCGCGCCGCCGTGTA
TCGCCCCGCCCTGGGCGGCAAGGCTGGCCCGGTCGGCACCAGTTGCGTGAGCGGAAAGATGGCCGCTTCCCGGCCCTGCTGCAGGGAG
CTCAAAATGGAGGACGCGGCGCTCGGGAGAGCGGGCGGGTGAGTCACCCACACAAAGGAAAAGGGCCTTTCCGTCCTCAGCCGTCGCT
TCATGTGACTCCACTGAGTACCGGGCGCCGTCCAGGCACCTCGATTAGTTCTCGTGCTTTTGGAGTACGTCGTCTTTAGGTTGGGGGG
AGGGGTTTTATGCGATGGAGTTTCCCCACACTGAGTGGGTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAGTGGTTCAAAGTTTTTTTCTTCCATTTCAGGTGTCGTGA
TTCTAGAGCCACCATGGTGAGCAAGGGCGAGGAGGATAACATGGCCATCATCAAGGAGTTCATGCGCTTCAAGGTGCACATGGAGGGC
TCCGTGAACGGCCACGAGTTCGAGATCGAGGGCGAGGGCGAGGGCCGCCCCTACGAGGGCACCCAGACCGCCAAGCTGAAGGTGACCA
AGGGTGGCCCCCTGCCCTTCGCCTGGGACATCCTGTCCCCTCAGTTCATGTACGGCTCCAAGGCCTACGTGAAGCACCCCGCCGACAT
CCCCGACTACTTGAAGCTGTCCTTCCCCGAGGGCTTCAAGTGGGAGCGCGTGATGAACTTCGAGGACGGCGGCGTGGTGACCGTGACC
CAGGACTCCTCCCTGCAGGACGGCGAGTTCATCTACAAGGTGAAGCTGCGCGGCACCAACTTCCCCTCCGACGGCCCCGTAATGCAGA
AGAAGACCATGGGCTGGGAGGCCTCCTCCGAGCGGATGTACCCCGAGGACGGCGCCCTGAAGGGCGAGATCAAGCAGAGGCTGAAGCT
GAAGGACGGCGGCCACTACGACGCTGAGGTCAAGACCACCTACAAGGCCAAGAAGCCCGTGCAGCTGCCCGGCGCCTACAACGTCAAC
ATCAAGTTGGACATCACCTCCCACAACGAGGACTACACCATCGTGGAACAGTACGAACGCGCCGAGGGCCGCCACTCCACCGGCGGCA
TGGACGAGCTGTACAAGAGGGCCAAGAGGCGTACGTAAG
6. pASP18 (orientation of inducible and constitutive modules: same direction)
(SEQ ID NO: 15)
TCGAGTTAATTAAGCTAGCCCGGTACTGTTGGAGGAAAAACTGTTTCATACAGAAGGCGTGGAGGAAAAACTGTTTCATACAGAAGGC
GTGGAGGAAAAACTGTTTCATACAGAAGGCGTGGAGGAAAAACTGTTTCATACAGAAGGCGTGGAGGAAAAACTGTTTCATACAGAAG
GCGTGGAGGAAAAACTGTTTCATACAGAAGGCGTACTAGTTAGAGGGTATATAATGGAAGCTCGACTTCCAGCCCGGGGGCATTCCGG
TACTGTTGGTAAAGCCACCATGTGCCACCAGCAGCTGGTCATCAGCTGGTTCAGCCTGGTGTTCCTGGCCAGCCCTCTGGTGGCCATC
TGGGAGCTGAAGAAAGACGTGTACGTGGTGGAACTGGACTGGTATCCCGACGCCCCTGGCGAGATGGTGGTGCTGACCTGCGACACCC
CTGAAGAGGACGGCATCACCTGGACCCTGGACCAGTCTAGCGAGGTGCTGGGCAGCGGCAAGACCCTGACCATCCAGGTCAAAGAGTT
CGGCGACGCCGGCCAGTACACCTGTCACAAGGGCGGAGAGGTGCTGAGCCACAGCCTGCTGCTGCTCCACAAGAAAGAGGATGGCATT
TGGAGCACCGACATCCTGAAGGACCAGAAAGAGCCCAAGAACAAGACCTTCCTGAGATGCGAGGCCAAGAACTACAGCGGCCGGTTCA
CCTGTTGGTGGCTGACCACCATCAGCACCGACCTGACCTTCAGCGTGAAGTCCAGCAGAGGCAGCAGCGACCCTCAGGGCGTGACATG
TGGCGCCGCTACACTGTCTGCCGAGAGAGTGCGGGGCGACAACAAAGAGTACGAGTACAGCGTCGAGTGCCAGGAAGATAGCGCCTGC
CCTGCCGCCGAGGAAAGCCTGCCTATCGAAGTGATGGTGGACGCCGTGCACAAGCTGAAGTACGAGAACTACACCTCCAGCTTTTTCA
TCCGGGACATCATCAAGCCCGACCCTCCCAAGAACCTGCAGCTGAAGCCTCTGAAGAACAGCAGACAGGTGGAAGTGTCCTGGGAGTA
CCCCGACACCTGGTCCACCCCTCACAGCTACTTCAGCCTGACATTCTGTGTGCAAGTCCAGGGCAAGTCCAAGCGCGAGAAAAAGGAC
CGGGTGTTCACCGACAAGACCAGCGCCACCGTGATCTGCCGGAAGAACGCCTCTATCAGCGTGCGGGCCCAGGACCGGTACTACAGCA
GCTCTTGGAGCGAGTGGGCCAGCGTGCCATGTTCTGGTGGCGGAGGCGGCGGAAGCAGAAATCTGCCAGTGGCCACCCCTGACCCCGG
CATGTTTCCTTGTCTGCACCACAGCCAGAACCTGCTGCGGGCCGTGTCCAACATGCTGCAGAAGGCCCGGCAGACCCTGGAATTCTAC
CCCTGCACCAGCGAGGAAATCGACCACGAGGACATCACCAAGGATAAGACCAGCACCGTGGAAGCCTGTCTGCCCCTGGAACTGACCA
AGAACGAGAGCTGCCTGAACAGCCGGGAAACCAGCTTCATCACCAACGGCAGCTGTCTGGCCAGCAGAAAGACCTCCTTCATGATGGC
CCTGTGCCTGAGCAGCATCTACGAGGACCTGAAGATGTACCAGGTCGAGTTCAAGACCATGAACGCCAAGCTGCTGATGGACCCCAAG
CGGCAGATCTTTCTGGACCAGAATATGCTGGCCGTGATCGACGAGCTGATGCAGGCCCTGAACTTCAACAGCGAGACAGTGCCCCAGA
AGTCCTCCCTGGAAGAACCCGACTTCTACAAGACCAAGATCAAGCTGTGCATCCTGCTGCACGCCTTCCGGATCAGAGCCGTGACCAT
CGACAGAGTGATGAGCTACCTGAACGCCTCCTAAATGCATCGTGAGGCTCCGGTGCCCGTCAGTGGGCAGAGCGCACATCGCCCACAG
TCCCCGAGAAGTTGGGGGGAGGGGTCGGCAATTGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGCCGTGAACGTTCTTTTTCGCAACGGGTTTGCC
GCCAGAACACAGGTAAGTGCCGTGTGTGGTTCCCGCGGGCCTGGCCTCTTTACGGGTTATGGCCCTTGCGTGCCTTGAATTACTTCCA
CCTGGCTGCAGTACGTGATTCTTGATCCCGAGCTTCGGGTTGGAAGTGGGTGGGAGAGTTCGAGGCCTTGCGCTTAAGGAGCCCCTTC
GCCTCGTGCTTGAGTTGAGGCCTGGCCTGGGCGCTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTGGCAAGATAGTCTTGTAAATGCGGGCCAAGATC
TGCACACTGGTATTTCGGTTTTTGGGGCCGCGGGCGGCGACGGGGCCCGTGCGTCCCAGCGCACATGTTCGGCGAGGCGGGGCCTGCG
AGCGCGGCCACCGAGAATCGGACGGGGGTAGTCTCAAGCTGGCCGGCCTGCTCTGGTGCCTGGCCTCGCGCCGCCGTGTATCGCCCCG
CCCTGGGCGGCAAGGCTGGCCCGGTCGGCACCAGTTGCGTGAGCGGAAAGATGGCCGCTTCCCGGCCCTGCTGCAGGGAGCTCAAAAT
GGAGGACGCGGCGCTCGGGAGAGCGGGCGGGTGAGTCACCCACACAAAGGAAAAGGGCCTTTCCGTCCTCAGCCGTCGCTTCATGTGA
CTCCACTGAGTACCGGGCGCCGTCCAGGCACCTCGATTAGTTCTCGTGCTTTTGGAGTACGTCGTCTTTAGGTTGGGGGGAGGGGTTT
TATGCGATGGAGTTTCCCCACACTGAGTGGGTGGAGACTGAAGTTAGGCCAGCTTGGCACTTGATGTAATTCTCCTTGGAATTTGCCC
TTTTTGAGTTTGGATCTTGGTTCATTCTCAAGCCTCAGACAGTGGTTCAAAGTTTTTTTCTTCCATTTCAGGTGTCGTGATTCTAGAG
CCACCATGGTGAGCAAGGGCGAGGAGGATAACATGGCCATCATCAAGGAGTTCATGCGCTTCAAGGTGCACATGGAGGGCTCCGTGAA
CGGCCACGAGTTCGAGATCGAGGGCGAGGGCGAGGGCCGCCCCTACGAGGGCACCCAGACCGCCAAGCTGAAGGTGACCAAGGGTGGC
CCCCTGCCCTTCGCCTGGGACATCCTGTCCCCTCAGTTCATGTACGGCTCCAAGGCCTACGTGAAGCACCCCGCCGACATCCCCGACT
ACTTGAAGCTGTCCTTCCCCGAGGGCTTCAAGTGGGAGCGCGTGATGAACTTCGAGGACGGCGGCGTGGTGACCGTGACCCAGGACTC
CTCCCTGCAGGACGGCGAGTTCATCTACAAGGTGAAGCTGCGCGGCACCAACTTCCCCTCCGACGGCCCCGTAATGCAGAAGAAGACC
ATGGGCTGGGAGGCCTCCTCCGAGCGGATGTACCCCGAGGACGGCGCCCTGAAGGGCGAGATCAAGCAGAGGCTGAAGCTGAAGGACG
GCGGCCACTACGACGCTGAGGTCAAGACCACCTACAAGGCCAAGAAGCCCGTGCAGCTGCCCGGCGCCTACAACGTCAACATCAAGTT
GGACATCACCTCCCACAACGAGGACTACACCATCGTGGAACAGTACGAACGCGCCGAGGGCCGCCACTCCACCGGCGGCATGGACGAG
CTGTACAAGAGGGCCAAGAGGCGTACGTAAG
7. pASP26 (orientation of inducible and constitutive modules: same direction)
(SEQ ID NO: 16)
TCGAGTTAATTAAGCTAGCCCGGTACTGTTGGAGGAAAAACTGTTTCATACAGAAGGCGTGGAGGAAAAACTGTTTCATACAGAAGGC
GTGGAGGAAAAACTGTTTCATACAGAAGGCGTGGAGGAAAAACTGTTTCATACAGAAGGCGTGGAGGAAAAACTGTTTCATACAGAAG
GCGTGGAGGAAAAACTGTTTCATACAGAAGGCGTACTAGTTAGAGGGTATATAATGGAAGCTCGACTTCCAGCCCGGGGGCATTCCGG
TACTGTTGGTAAAGCCACCATGGTGAGCAAGGGCGAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGACGACGGCAACTACAAGACCCGCGCC
GAGGTGAAGTTCGAGGGCGACACCCTGGTGAACCGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAAGAACGGCATCAAGGTGAACTTCAAGATCCGCCA
CAACATCGAGGACGGCAGCGTGCAGCTCGCCGACCACTACCAGCAGAACACCCCCATCGGCGACGGCCCCGTGCTGCTGCCCGACAAC
CACTACCTGAGCACCCAGTCCGCCCTGAGCAAAGACCCCAACGAGAAGCGCGATCACATGGTCCTGCTGGAGTTCGTGACCGCCGCCG
GGATCACTCTCGGCATGGACGAGCTGTACAAGTAAATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
GCCACCATGGCCTTACCAGTGACCGCCTTGCTCCTGCCGCTGGCCTTGCTGCTCCACGCCGCCAGGCCGGACATTCAAATGACACAGA
GTCCCTCATCCCTCAGTGCCAGCGTGGGCGATCGGGTGACTATAACCTGCAGAGCTTCTCAGGACGTGAATACCGCTGTGGCGTGGTA
CCAGCAGAAGCCAGGCAAAGCGCCTAAGCTTCTCATTTATAGTGCCAGCTTCCTGTACTCAGGTGTTCCGTCTCGCTTTTCTGGAAGT
AGAAGTGGGACCGATTTCACATTGACGATCAGCAGCTTGCAGCCCGAAGATTTCGCCACCTACTACTGTCAGCAGCACTACACTACCC
CACCGACATTTGGTCAAGGCACAAAAGTAGAGATTAAACGCACTGGTTCCACCAGCGGGAGCGGGAAACCCGGCTCTGGGGAGGGGAG
CGAGGTCCAGCTGGTGGAATCCGGGGGTGGTCTTGTGCAGCCAGGAGGATCCTTGAGGTTGTCCTGCGCCGCAAGCGGCTTTAACATC
AAAGATACATACATCCATTGGGTCCGACAGGCCCCTGGAAAGGGCCTGGAGTGGGTCGCCCGGATCTACCCAACTAACGGGTACACTC
GCTACGCTGATAGCGTCAAGGGTCGGTTTACTATTTCTGCCGACACCTCAAAAAACACAGCCTACCTCCAGATGAACTCTCTCAGAGC
TGAGGATACAGCCGTGTACTATTGCAGCCGGTGGGGAGGCGACGGGTTCTACGCTATGGATGTGTGGGGGCAGGGCACACTGGTCACC
GTGAGCTCATCCGGAACCACGACGCCAGCGCCGCGACCACCAACACCGGCGCCCACCATCGCGTCGCAGCCCCTGTCCCTGCGCCCAG
AGGCGTGCCGGCCAGCGGCGGGGGGCGCAGTGCACACGAGGGGGCTGGACTTCGCCTGTGATATCTACATCTGGGCGCCCTTGGCCGG
GACTTGTGGGGTCCTTCTCCTGTCACTGGTTATCACCCTTTACTGCAAACGGGGCAGAAAGAAACTCCTGTATATATTCAAACAACCA
TTTATGAGACCAGTACAAACTACTCAAGAGGAAGATGGCTGTAGCTGCCGATTTCCAGAAGAAGAAGAAGGAGGATGTGAACTGAGAG
TGAAGTTCAGCAGGAGCGCAGACGCCCCCGCGTACAAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCCCCTCGCTAAG
8. pASP28 (orientation of inducible and constitutive modules: opposite direction)
(SEQ ID NO: 17)
TCGAGTTAATTAAGCTAGCTTACTTGTACAGCTCGTCCATGCCGAGAGTGATCCCGGCGGCGGTCACGAACTCCAGCAGGACCATGTG
ATCGCGCTTCTCGTTGGGGTCTTTGCTCAGGGCGGACTGGGTGCTCAGGTAGTGGTTGTCGGGCAGCAGCACGGGGCCGTCGCCGATG
GGGGTGTTCTGCTGGTAGTGGTCGGCGAGCTGCACGCTGCCGTCCTCGATGTTGTGGCGGATCTTGAAGTTCACCTTGATGCCGTTCT
TCTGCTTGTCGGCCATGATATAGACGTTGTGGCTGTTGTAGTTGTACTCCAGCTTGTGCCCCAGGATGTTGCCGTCCTCCTTGAAGTC
GATGCCCTTCAGCTCGATGCGGTTCACCAGGGTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTGCTGCTTCATGTGGTCGGGGTAGCGGCTGAAGC
ACTGCACGCCGTAGGTCAGGGTGGTCACGAGGGTGGGCCAGGGCACGGGCAGCTTGCCGGTGGTGCAGATGAACTTCAGGGTCAGCTT
GCCGTAGGTGGCATCGCCCTCGCCCTCGCCGGACACGCTGAACTTGTGGCCGTTTACGTCGCCGTCCAGCTCGACCAGGATGGGCACC
ACCCCGGTGAACAGCTCCTCGCCCTTGCTCACCATGGTGGCTTTACCAACAGTACCGGAATGCCCCCGGGCTGGAAGTCGAGCTTCCA
TTATATACCCTCTAACTAGTACGCCTTCTGTATGAAACAGTTTTTCCTCCACGCCTTCTGTATGAAACAGTTTTTCCTCCACGCCTTC
TGTATGAAACAGTTTTTCCTCCACGCCTTCTGTATGAAACAGTTTTTCCTCCACGCCTTCTGTATGAAACAGTTTTTCCTCCACGCCT
TCTGTATGAAACAGTTTTTCCTCCAACAGTACCGGATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
ATGGCCTTACCAGTGACCGCCTTGCTCCTGCCGCTGGCCTTGCTGCTCCACGCCGCCAGGCCGGGATCCGACATCGTTCTGAGTCAGA
GCCCCGCCATACTGAGCGCCTCCCCCGGGGAGAAGGTGACCATGACCTGTAGAGCCAGCTCCAGCCTTTCATTTATGCACTGGTACCA
GCAGAAACCCGGTAGTAGTCCAAAGCCGTGGATTTACGCTACATCCAACCTGGCAAGCGGAGTACCAGCCCGCTTCTCCGGCAGCGGA
TCAGGAACCTCCTACTCCCTGACAATCAGCCGCGTCGAAGCCGAGGATGCAGCCACATACTTTTGCCACCAGTGGTCCTCTAATCCCC
TGACATTTGGCGCAGGCACCAAGCTCGAACTGAAGCGGGGATCTACCAGCGGCAGTGGAAAACCTGGTAGCGGAGAGGGGTCTACCAA
GGGGCAAGTGCAACTCAGACAGCCTGGCGCAGAACTCGTGAAGCCCGGCGCATCCGTCAAGATGTCTTGTAAAGCCAGTGGCTACACC
TTCACTAGCTATAATATGCACTGGGTTAAACAGACTCCCGGTCAGGGCCTCGAGTGGATTGGCGCAATCTACCCGGGTAATGGTGATA
CTTCTTACAATCAGAAGTTTAAGGGGAAAGCTACCCTGACCGCCGACAAGTCTTCATCAACCGCCTACATGCAGCTGAGCTCTCTGAC
GTCTGAGGATTCCGCCGTGTATTACTGCGCCCGGAGCCACTACGGGAGTAATTATGTCGATTATTTTGACTACTGGGGTCAGGGAACA
ACCCTGACTGTCTCCAGCTCCGGAACCACGACGCCAGCGCCGCGACCACCAACACCGGCGCCCACCATCGCGTCGCAGCCCCTGTCCC
TGCGCCCAGAGGCGTGCCGGCCAGCGGCGGGGGGCGCAGTGCACACGAGGGGGCTGGACTTCGCCTGTGATATCTACATCTGGGCGCC
CTTGGCCGGGACTTGTGGGGTCCTTCTCCTGTCACTGGTTATCACCCTTTACTGCAAACGGGGCAGAAAGAAACTCCTGTATATATTC
AAACAACCATTTATGAGACCAGTACAAACTACTCAAGAGGAAGATGGCTGTAGCTGCCGATTTCCAGAAGAAGAAGAAGGAGGATGTG
AACTGAGAGTGAAGTTCAGCAGGAGCGCAGACGCCCCCGCGTACAAGCAGGGCCAGAACCAGCTCTATAACGAGCTCAATCTAGGACG
AAGAGAGGAGTACGATGTTTTGGACAAGAGACGTGGCCGGGACCCTGAGATGGGGGGAAAGCCGAGAAGGAAGAACCCTCAGGAAGGC
CTGTACAATGAACTGCAGAAAGATAAGATGGCGGAGGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCACATGCAGGCCCTGCCCCCTCGCTAAG
9. pASP30 (orientation of inducible and constitutive modules: opposite direction)
(SEQ ID NO: 18)
TCGAGTTAATTAAGCTAGCTTACTTGTACAGCTCGTCCATGCCGAGAGTGATCCCGGCGGCGGTCACGAACTCCAGCAGGACCATGTG
ATCGCGCTTCTCGTTGGGGTCTTTGCTCAGGGCGGACTGGGTGCTCAGGTAGTGGTTGTCGGGCAGCAGCACGGGGCCGTCGCCGATG
GGGGTGTTCTGCTGGTAGTGGTCGGCGAGCTGCACGCTGCCGTCCTCGATGTTGTGGCGGATCTTGAAGTTCACCTTGATGCCGTTCT
TCTGCTTGTCGGCCATGATATAGACGTTGTGGCTGTTGTAGTTGTACTCCAGCTTGTGCCCCAGGATGTTGCCGTCCTCCTTGAAGTC
GATGCCCTTCAGCTCGATGCGGTTCACCAGGGTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTGCTGCTTCATGTGGTCGGGGTAGCGGCTGAAGC
ACTGCACGCCGTAGGTCAGGGTGGTCACGAGGGTGGGCCAGGGCACGGGCAGCTTGCCGGTGGTGCAGATGAACTTCAGGGTCAGCTT
GCCGTAGGTGGCATCGCCCTCGCCCTCGCCGGACACGCTGAACTTGTGGCCGTTTACGTCGCCGTCCAGCTCGACCAGGATGGGCACC
ACCCCGGTGAACAGCTCCTCGCCCTTGCTCACCATGGTGGCTTTACCAACAGTACCGGAATGCCCCCGGGCTGGAAGTCGAGCTTCCA
TTATATACCCTCTAACTAGTACGCCTTCTGTATGAAACAGTTTTTCCTCCACGCCTTCTGTATGAAACAGTTTTTCCTCCACGCCTTC
TGTATGAAACAGTTTTTCCTCCACGCCTTCTGTATGAAACAGTTTTTCCTCCACGCCTTCTGTATGAAACAGTTTTTCCTCCACGCCT
TCTGTATGAAACAGTTTTTCCTCCAACAGTACCGGATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
GCCACCATGGCCTTACCAGTGACCGCCTTGCTCCTGCCGCTGGCCTTGCTGCTCCACGCCGCCAGGCCGGACATTCAAATGACACAGA
GTCCCTCATCCCTCAGTGCCAGCGTGGGCGATCGGGTGACTATAACCTGCAGAGCTTCTCAGGACGTGAATACCGCTGTGGCGTGGTA
CCAGCAGAAGCCAGGCAAAGCGCCTAAGCTTCTCATTTATAGTGCCAGCTTCCTGTACTCAGGTGTTCCGTCTCGCTTTTCTGGAAGT
AGAAGTGGGACCGATTTCACATTGACGATCAGCAGCTTGCAGCCCGAAGATTTCGCCACCTACTACTGTCAGCAGCACTACACTACCC
CACCGACATTTGGTCAAGGCACAAAAGTAGAGATTAAACGCACTGGTTCCACCAGCGGGAGCGGGAAACCCGGCTCTGGGGAGGGGAG
CGAGGTCCAGCTGGTGGAATCCGGGGGTGGTCTTGTGCAGCCAGGAGGATCCTTGAGGTTGTCCTGCGCCGCAAGCGGCTTTAACATC
AAAGATACATACATCCATTGGGTCCGACAGGCCCCTGGAAAGGGCCTGGAGTGGGTCGCCCGGATCTACCCAACTAACGGGTACACTC
GCTACGCTGATAGCGTCAAGGGTCGGTTTACTATTTCTGCCGACACCTCAAAAAACACAGCCTACCTCCAGATGAACTCTCTCAGAGC
TGAGGATACAGCCGTGTACTATTGCAGCCGGTGGGGAGGCGACGGGTTCTACGCTATGGATGTGTGGGGGCAGGGCACACTGGTCACC
GTGAGCTCATCCGGAACCACGACGCCAGCGCCGCGACCACCAACACCGGCGCCCACCATCGCGTCGCAGCCCCTGTCCCTGCGCCCAG
AGGCGTGCCGGCCAGCGGCGGGGGGCGCAGTGCACACGAGGGGGCTGGACTTCGCCTGTGATATCTACATCTGGGCGCCCTTGGCCGG
GACTTGTGGGGTCCTTCTCCTGTCACTGGTTATCACCCTTTACTGCAAACGGGGCAGAAAGAAACTCCTGTATATATTCAAACAACCA
TTTATGAGACCAGTACAAACTACTCAAGAGGAAGATGGCTGTAGCTGCCGATTTCCAGAAGAAGAAGAAGGAGGATGTGAACTGAGAG
TGAAGTTCAGCAGGAGCGCAGACGCCCCCGCGTACAAGCAGGGCCAGAACCAGCTCTATAACGAGCTCAATCTAGGACGAAGAGAGGA
GTACGATGTTTTGGACAAGAGACGTGGCCGGGACCCTGAGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCCCCTCGCTAAG
10. pASP31 (orientation of inducible and constitutive modules: opposite direction)
(SEQ ID NO: 19)
TCGAGTTAATTAAGCTAGCTTACTTGTACAGCTCGTCCATGCCGAGAGTGATCCCGGCGGCGGTCACGAACTCCAGCAGGACCATGTG
ATCGCGCTTCTCGTTGGGGTCTTTGCTCAGGGCGGACTGGGTGCTCAGGTAGTGGTTGTCGGGCAGCAGCACGGGGCCGTCGCCGATG
GGGGTGTTCTGCTGGTAGTGGTCGGCGAGCTGCACGCTGCCGTCCTCGATGTTGTGGCGGATCTTGAAGTTCACCTTGATGCCGTTCT
TCTGCTTGTCGGCCATGATATAGACGTTGTGGCTGTTGTAGTTGTACTCCAGCTTGTGCCCCAGGATGTTGCCGTCCTCCTTGAAGTC
GATGCCCTTCAGCTCGATGCGGTTCACCAGGGTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTGCTGCTTCATGTGGTCGGGGTAGCGGCTGAAGC
ACTGCACGCCGTAGGTCAGGGTGGTCACGAGGGTGGGCCAGGGCACGGGCAGCTTGCCGGTGGTGCAGATGAACTTCAGGGTCAGCTT
GCCGTAGGTGGCATCGCCCTCGCCCTCGCCGGACACGCTGAACTTGTGGCCGTTTACGTCGCCGTCCAGCTCGACCAGGATGGGCACC
ACCCCGGTGAACAGCTCCTCGCCCTTGCTCACCATGGTGGCTTTACCAACAGTACCGGAATGCCCCCGGGCTGGAAGTCGAGCTTCCA
TTATATACCCTCTAACTAGTACGCCTTCTGTATGAAACAGTTTTTCCTCCACGCCTTCTGTATGAAACAGTTTTTCCTCCACGCCTTC
TGTATGAAACAGTTTTTCCTCCACGCCTTCTGTATGAAACAGTTTTTCCTCCACGCCTTCTGTATGAAACAGTTTTTCCTCCACGCCT
TCTGTATGAAACAGTTTTTCCTCCAACAGTACCGGATGCATCGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGTAAACTGGGAAAGTGATGTCGTGT
ACTGGCTCCGCCTTTTTCCCGAGGGTGGGGGAGAACCGTATATAAGTGCAGTAGTCGCCGTGAACGTTCTTTTTCGCAACGGGTTTGC
CGCCAGAACACAGGTAAGTGCCGTGTGTGGTTCCCGCGGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAATCTGGTGGCACCTTCGCGCCTGTCTCGCTGCTTT
CGATAAGTCTCTAGCCATTTAAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTGGTGCCTGGCCTCGCGCCGCCGTGTATCGCCCC
GCCCTGGGCGGCAAGGCTGGCCCGGTCGGCACCAGTTGCGTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAAGGAAAAGGGCCTTTCCGTCCTCAGCCGTCGCTTCATGTG
ACTCCACTGAGTACCGGGCGCCGTCCAGGCACCTCGATTAGTTCTCGTGCTTTTGGAGTACGTCGTCTTTAGGTTGGGGGGAGGGGTT
TTATGCGATGGAGTTTCCCCACACTGAGTGGGTGGAGACTGAAGTTAGGCCAGCTTGGCACTTGATGTAATTCTCCTTGGAATTTGCC
CTTTTTGAGTTTGGATCTTGGTTCATTCTCAAGCCTCAGACAGTGGTTCAAAGTTTTTTTCTTCCATTTCAGGTGTCGTGATTCTAGA
GCCACCATGGCCTTACCAGTGACCGCCTTGCTCCTGCCGCTGGCCTTGCTGCTCCACGCCGCCAGGCCGGACATTCAAATGACACAGA
GTCCCTCATCCCTCAGTGCCAGCGTGGGCGATCGGGTGACTATAACCTGCAGAGCTTCTCAGGACGTGAATACCGCTGTGGCGTGGTA
CCAGCAGAAGCCAGGCAAAGCGCCTAAGCTTCTCATTTATAGTGCCAGCTTCCTGTACTCAGGTGTTCCGTCTCGCTTTTCTGGAAGT
AGAAGTGGGACCGATTTCACATTGACGATCAGCAGCTTGCAGCCCGAAGATTTCGCCACCTACTACTGTCAGCAGCACTACACTACCC
CACCGACATTTGGTCAAGGCACAAAAGTAGAGATTAAACGCACTGGTTCCACCAGCGGGAGCGGGAAACCCGGCTCTGGGGAGGGGAG
CGAGGTCCAGCTGGTGGAATCCGGGGGTGGTCTTGTGCAGCCAGGAGGATCCTTGAGGTTGTCCTGCGCCGCAAGCGGCTTTAACATC
AAAGATACATACATCCATTGGGTCCGACAGGCCCCTGGAAAGGGCCTGGAGTGGGTCGCCCGGATCTACCCAACTAACGGGTACACTC
GCTACGCTGATAGCGTCAAGGGTCGGTTTACTATTTCTGCCGACACCTCAAAAAACACAGCCTACCTCCAGATGAACTCTCTCAGAGC
TGAGGATACAGCCGTGTACTATTGCAGCCGGTGGGGAGGCGACGGGTTCTACGCTATGGATGTGTGGGGGCAGGGCACACTGGTCACC
GTGAGCTCATCCGGAACCACGACGCCAGCGCCGCGACCACCAACACCGGCGCCCACCATCGCGTCGCAGCCCCTGTCCCTGCGCCCAG
AGGCGTGCCGGCCAGCGGCGGGGGGCGCAGTGCACACGAGGGGGCTGGACTTCGCCTGTGATTTTTGGGTGCTGGTGGTGGTTGGTGG
AGTCCTGGCTTGCTATAGCTTGCTAGTAACAGTGGCCTTTATTATTTTCTGGGTGAGGAGTAAGAGGAGCAGGCTCCTGCACAGTGAC
TACATGAACATGACTCCCCGCCGCCCCGGGCCCACCCGCAAGCATTACCAGCCCTATGCCCCACCACGCGACTTCGCAGCCTATCGCT
CCATCGATAGAGTGAAGTTCAGCAGGAGCGCAGACGCCCCCGCGTACCAGCAGGGCCAGAACCAGCTCTATAACGAGCTCAATCTAGG
ACGAAGAGAGGAGTACGATGTTTTGGACAAGAGACGTGGCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCAGGCCCTGCCCCCTCGCTAAG
11. pASP38 (orientation of inducible and constitutive modules: same direction)
(SEQ ID NO: 20)
TCGAGTTAATTAAGCTAGCCCGGTACTGTTGGAGGAAAAACTGTTTCATACAGAAGGCGTGGAGGAAAAACTGTTTCATACAGAAGGC
GTGGAGGAAAAACTGTTTCATACAGAAGGCGTGGAGGAAAAACTGTTTCATACAGAAGGCGTGGAGGAAAAACTGTTTCATACAGAAG
GCGTGGAGGAAAAACTGTTTCATACAGAAGGCGTACTAGTTAGAGGGTATATAATGGAAGCTCGACTTCCAGCCCGGGGGCATTCCGG
TACTGTTGGTAAAGCCACCATGTGCCACCAGCAGCTGGTCATCAGCTGGTTCAGCCTGGTGTTCCTGGCCAGCCCTCTGGTGGCCATC
TGGGAGCTGAAGAAAGACGTGTACGTGGTGGAACTGGACTGGTATCCCGACGCCCCTGGCGAGATGGTGGTGCTGACCTGCGACACCC
CTGAAGAGGACGGCATCACCTGGACCCTGGACCAGTCTAGCGAGGTGCTGGGCAGCGGCAAGACCCTGACCATCCAGGTCAAAGAGTT
CGGCGACGCCGGCCAGTACACCTGTCACAAGGGCGGAGAGGTGCTGAGCCACAGCCTGCTGCTGCTCCACAAGAAAGAGGATGGCATT
TGGAGCACCGACATCCTGAAGGACCAGAAAGAGCCCAAGAACAAGACCTTCCTGAGATGCGAGGCCAAGAACTACAGCGGCCGGTTCA
CCTGTTGGTGGCTGACCACCATCAGCACCGACCTGACCTTCAGCGTGAAGTCCAGCAGAGGCAGCAGCGACCCTCAGGGCGTGACATG
TGGCGCCGCTACACTGTCTGCCGAGAGAGTGCGGGGCGACAACAAAGAGTACGAGTACAGCGTCGAGTGCCAGGAAGATAGCGCCTGC
CCTGCCGCCGAGGAAAGCCTGCCTATCGAAGTGATGGTGGACGCCGTGCACAAGCTGAAGTACGAGAACTACACCTCCAGCTTTTTCA
TCCGGGACATCATCAAGCCCGACCCTCCCAAGAACCTGCAGCTGAAGCCTCTGAAGAACAGCAGACAGGTGGAAGTGTCCTGGGAGTA
CCCCGACACCTGGTCCACCCCTCACAGCTACTTCAGCCTGACATTCTGTGTGCAAGTCCAGGGCAAGTCCAAGCGCGAGAAAAAGGAC
CGGGTGTTCACCGACAAGACCAGCGCCACCGTGATCTGCCGGAAGAACGCCTCTATCAGCGTGCGGGCCCAGGACCGGTACTACAGCA
GCTCTTGGAGCGAGTGGGCCAGCGTGCCATGTTCTGGTGGCGGAGGCGGCGGAAGCAGAAATCTGCCAGTGGCCACCCCTGACCCCGG
CATGTTTCCTTGTCTGCACCACAGCCAGAACCTGCTGCGGGCCGTGTCCAACATGCTGCAGAAGGCCCGGCAGACCCTGGAATTCTAC
CCCTGCACCAGCGAGGAAATCGACCACGAGGACATCACCAAGGATAAGACCAGCACCGTGGAAGCCTGTCTGCCCCTGGAACTGACCA
AGAACGAGAGCTGCCTGAACAGCCGGGAAACCAGCTTCATCACCAACGGCAGCTGTCTGGCCAGCAGAAAGACCTCCTTCATGATGGC
CCTGTGCCTGAGCAGCATCTACGAGGACCTGAAGATGTACCAGGTCGAGTTCAAGACCATGAACGCCAAGCTGCTGATGGACCCCAAG
CGGCAGATCTTTCTGGACCAGAATATGCTGGCCGTGATCGACGAGCTGATGCAGGCCCTGAACTTCAACAGCGAGACAGTGCCCCAGA
AGTCCTCCCTGGAAGAACCCGACTTCTACAAGACCAAGATCAAGCTGTGCATCCTGCTGCACGCCTTCCGGATCAGAGCCGTGACCAT
CGACAGAGTGATGAGCTACCTGAACGCCTCCTAAATGCATCGTGAGGCTCCGGTGCCCGTCAGTGGGCAGAGCGCACATCGCCCACAG
TCCCCGAGAAGTTGGGGGGAGGGGTCGGCAATTGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGCCGTGAACGTTCTTTTTCGCAACGGGTTTGCC
GCCAGAACACAGGTAAGTGCCGTGTGTGGTTCCCGCGGGCCTGGCCTCTTTACGGGTTATGGCCCTTGCGTGCCTTGAATTACTTCCA
CCTGGCTGCAGTACGTGATTCTTGATCCCGAGCTTCGGGTTGGAAGTGGGTGGGAGAGTTCGAGGCCTTGCGCTTAAGGAGCCCCTTC
GCCTCGTGCTTGAGTTGAGGCCTGGCCTGGGCGCTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTGGCAAGATAGTCTTGTAAATGCGGGCCAAGATC
TGCACACTGGTATTTCGGTTTTTGGGGCCGCGGGCGGCGACGGGGCCCGTGCGTCCCAGCGCACATGTTCGGCGAGGCGGGGCCTGCG
AGCGCGGCCACCGAGAATCGGACGGGGGTAGTCTCAAGCTGGCCGGCCTGCTCTGGTGCCTGGCCTCGCGCCGCCGTGTATCGCCCCG
CCCTGGGCGGCAAGGCTGGCCCGGTCGGCACCAGTTGCGTGAGCGGAAAGATGGCCGCTTCCCGGCCCTGCTGCAGGGAGCTCAAAAT
GGAGGACGCGGCGCTCGGGAGAGCGGGCGGGTGAGTCACCCACACAAAGGAAAAGGGCCTTTCCGTCCTCAGCCGTCGCTTCATGTGA
CTCCACTGAGTACCGGGCGCCGTCCAGGCACCTCGATTAGTTCTCGTGCTTTTGGAGTACGTCGTCTTTAGGTTGGGGGGAGGGGTTT
TATGCGATGGAGTTTCCCCACACTGAGTGGGTGGAGACTGAAGTTAGGCCAGCTTGGCACTTGATGTAATTCTCCTTGGAATTTGCCC
TTTTTGAGTTTGGATCTTGGTTCATTCTCAAGCCTCAGACAGTGGTTCAAAGTTTTTTTCTTCCATTTCAGGTGTCGTGATTCTAGAG
CCACCATGGCCTTACCAGTGACCGCCTTGCTCCTGCCGCTGGCCTTGCTGCTCCACGCCGCCAGGCCGGACATTCAAATGACACAGAG
TCCCTCATCCCTCAGTGCCAGCGTGGGCGATCGGGTGACTATAACCTGCAGAGCTTCTCAGGACGTGAATACCGCTGTGGCGTGGTAC
CAGCAGAAGCCAGGCAAAGCGCCTAAGCTTCTCATTTATAGTGCCAGCTTCCTGTACTCAGGTGTTCCGTCTCGCTTTTCTGGAAGTA
GAAGTGGGACCGATTTCACATTGACGATCAGCAGCTTGCAGCCCGAAGATTTCGCCACCTACTACTGTCAGCAGCACTACACTACCCC
ACCGACATTTGGTCAAGGCACAAAAGTAGAGATTAAACGCACTGGTTCCACCAGCGGGAGCGGGAAACCCGGCTCTGGGGAGGGGAGC
GAGGTCCAGCTGGTGGAATCCGGGGGTGGTCTTGTGCAGCCAGGAGGATCCTTGAGGTTGTCCTGCGCCGCAAGCGGCTTTAACATCA
AAGATACATACATCCATTGGGTCCGACAGGCCCCTGGAAAGGGCCTGGAGTGGGTCGCCCGGATCTACCCAACTAACGGGTACACTCG
CTACGCTGATAGCGTCAAGGGTCGGTTTACTATTTCTGCCGACACCTCAAAAAACACAGCCTACCTCCAGATGAACTCTCTCAGAGCT
GAGGATACAGCCGTGTACTATTGCAGCCGGTGGGGAGGCGACGGGTTCTACGCTATGGATGTGTGGGGGCAGGGCACACTGGTCACCG
TGAGCTCATCCGGAACCACGACGCCAGCGCCGCGACCACCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACTTCGCCTGTGATATCTACATCTGGGCGCCCTTGGCCGGG
ACTTGTGGGGTCCTTCTCCTGTCACTGGTTATCACCCTTTACTGCAAACGGGGCAGAAAGAAACTCCTGTATATATTCAAACAACCAT
TTATGAGACCAGTACAAACTACTCAAGAGGAAGATGGCTGTAGCTGCCGATTTCCAGAAGAAGAAGAAGGAGGATGTGAACTGAGAGT
GAAGTTCAGCAGGAGCGCAGACGCCCCCGCGTACAAGCAGGGCCAGAACCAGCTCTATAACGAGCTCAATCTAGGACGAAGAGAGGAG
TACGATGTTTTGGACAAGAGACGTGGCCGGGACCCTGAGATGGGGGGAAAGCCGAGAAGGAAGAACCCTCAGGAAGGCCTGTACAATG
AACTGCAGAAAGATAAGATGGCGGAGGCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCCCTCGCTAAG
12. pASP52 (orientation of inducible and constitutive modules: opposite direction)
(SEQ ID NO: 21)
TCGAGTTAATTAAGCTAGCTTACATATGTCTCTTGGCCCGCAGGTCCTCTTCGGAGATCAGCTTCTGCTCGCTTCCCTTGCCAGGAGA
CAGGCTCAGGGACTTCTGGGTGTAGTGGTTGTGCAGGGCCTCGTGCATCACGCTGCAGGAGAACACGTTTCCCTGCTGCCATCTGCTC
TTGTCCACTGTCAGCTTGCTGTACAGGAAGAATGAGCCGTCGCTGTCCAGCACAGGAGGGGTTGTCTTGTAGTTGTTCTCAGGCTGGC
CATTGCTCTCCCATTCCACGGCAATGTCGCTGGGGTAAAAGCCCTTGACCAGGCAGGTCAGGGACACCTGATTCTTGGTCAGCTCGTC
CCTGCTTGGAGGCAGTGTGTAAACCTGGGGTTCCCTAGGCTGGCCCTTGGCCTTGCTGATGGTTTTCTCGATAGGAGCAGGCAGGGCC
TTGTTGGACACCTTGCACTTGTACTCTTTGCCGTTCAGCCAATCCTGGTGCAGCACGGTCAGCACGGACACCACTCTGTAGGTGCTGT
TGTACTGTTCCTCTCTAGGCTTGGTCTTGGCGTTGTGCACTTCCACGCCGTCCACGTACCAATTGAACTTCACTTCGGGGTCCTCGTG
AGACACATCCACCACCACGCAGGTCACTTCAGGGGTTCTGCTGATCATCAGGGTGTCCTTAGGCTTTGGAGGAAACAGGAACACGGAA
GGTCCGCCGAGCAGTTCTGGAGCAGGACATGGAGGACAGGTGTGGGTCTTGTCGCTGCTCTTGGGTTCCACTTTGATTTCCACCTTGG
TGCCCTGGCCAAATGTGTAAGGCAGGGTGTTGCCCTGCTGGCAGTAGTAGGTGGCAATGTCCTCAGGCTGCAGGCTAGATATGGTGAA
GGTGAAGTCGGTGCCGCTGCCAGAGCCAGAAAATCTGCTGGGCACGCCGCTGTGCAGTCTGCTGGTGTAGTAGATCAGCAGTTTAGGG
GCCTTGCCGGGCTTTTGCTGATACCAATTCAGGTAGCTGCTGATGTCCTGGCTGGCCCGACAGGTGATTGTCACTCTGTCTCCCACAG
AGGCAGACAGGCTGCTGGGGCTCTGTGTCATCTGAATATCGCTACCGCCTCCGCCACTTCCTCCGCCACCAGATCCTCCGCCTCCGCT
AGAAACGGTGACCAGAGAGCCCTGGCCCCAATAATCCATGGCGGTGGTTCTGGCCAGGCTTCTGGCGCAGTAGTACACGGCTGTATCA
GCAGCGGTCACGCTAGACAGCCGCAGGCTGAACTGGTTCTTGCTGGTGTCTCTCAGCATGGTCACTCTGGACTTCAGGCTGGGATTGT
AGGTGGTGATGCCGCTGTAGCTGATGTAGCCGATCCATTCCAGGCCTCTTCCAGGAGGTTGTCGGACCCAGGACCAGGCATGATCGCT
GGTGATGCTGTAGCCGGACACGGTACAGGTCAGGCTCAGTGTTTGGCTAGGCCGCACGAGTCCAGGTCCAGACTCTTGCAGCTGAACC
TCGCACTGCACGCCTCTAAGCAGAGCCACCAGGAACACCCAGCTCAGGCCGAATTCCATGGTGGCTTTACCAACAGTACCGGAATGCC
CCCGGGCTGGAAGTCGAGCTTCCATTATATACCCTCTAACTAGTACGCCTTCTGTATGAAACAGTTTTTCCTCCACGCCTTCTGTATG
AAACAGTTTTTCCTCCACGCCTTCTGTATGAAACAGTTTTTCCTCCACGCCTTCTGTATGAAACAGTTTTTCCTCCACGCCTTCTGTA
TGAAACAGTTTTTCCTCCACGCCTTCTGTATGAAACAGTTTTTCCTCCAACAGTACCGGATGCATCGTGAGGCTCCGGTGCCCGTCAG
TGGGCAGAGCGCACATCGCCCACAGTCCCCGAGAAGTTGGGGGGAGGGGTCGGCAATTGAACCGGTGCCTAGAGAAGGTGGCGCGGGG
TAAACTGGGAAAGTGATGTCGTGTACTGGCTCCGCCTTTTTCCCGAGGGTGGGGGAGAACCGTATATAAGTGCAGTAGTCGCCGTGAA
CGTTCTTTTTCGCAACGGGTTTGCCGCCAGAACACAGGTAAGTGCCGTGTGTGGTTCCCGCGGGCCTGGCCTCTTTACGGGTTATGGC
CCTTGCGTGCCTTGAATTACTTCCACCTGGCTGCAGTACGTGATTCTTGATCCCGAGCTTCGGGTTGGAAGTGGGTGGGAGAGTTCGA
GGCCTTGCGCTTAAGGAGCCCCTTCGCCTCGTGCTTGAGTTGAGGCCTGGCCTGGGCGCTGGGGCCGCCGCGTGCGAATCTGGTGGCA
CCTTCGCGCCTGTCTCGCTGCTTTCGATAAGTCTCTAGCCATTTAAAATTTTTGATGACCTGCTGCGACGCTTTTTTTCTGGCAAGAT
AGTCTTGTAAATGCGGGCCAAGATCTGCACACTGGTATTTCGGTTTTTGGGGCCGCGGGCGGCGACGGGGCCCGTGCGTCCCAGCGCA
CATGTTCGGCGAGGCGGGGCCTGCGAGCGCGGCCACCGAGAATCGGACGGGGGTAGTCTCAAGCTGGCCGGCCTGCTCTGGTGCCTGG
CCTCGCGCCGCCGTGTATCGCCCCGCCCTGGGCGGCAAGGCTGGCCCGGTCGGCACCAGTTGCGTGAGCGGAAAGATGGCCGCTTCCC
GGCCCTGCTGCAGGGAGCTCAAAATGGAGGACGCGGCGCTCGGGAGAGCGGGCGGGTGAGTCACCCACACAAAGGAAAAGGGCCTTTC
CGTCCTCAGCCGTCGCTTCATGTGACTCCACTGAGTACCGGGCGCCGTCCAGGCACCTCGATTAGTTCTCGTGCTTTTGGAGTACGTC
GTCTTTAGGTTGGGGGGAGGGGTTTTATGCGATGGAGTTTCCCCACACTGAGTGGGTGGAGACTGAAGTTAGGCCAGCTTGGCACTTG
ATGTAATTCTCCTTGGAATTTGCCCTTTTTGAGTTTGGATCTTGGTTCATTCTCAAGCCTCAGACAGTGGTTCAAAGTTTTTTTCTTC
CATTTCAGGTGTCGTGATTCTAGAATGGCCTTACCAGTGACCGCCTTGCTCCTGCCGCTGGCCTTGCTGCTCCACGCCGCCAGGCCGG
GATCCGACATCGTTCTGAGTCAGAGCCCCGCCATACTGAGCGCCTCCCCCGGGGAGAAGGTGACCATGACCTGTAGAGCCAGCTCCAG
CCTTTCATTTATGCACTGGTACCAGCAGAAACCCGGTAGTAGTCCAAAGCCGTGGATTTACGCTACATCCAACCTGGCAAGCGGAGTA
CCAGCCCGCTTCTCCGGCAGCGGATCAGGAACCTCCTACTCCCTGACAATCAGCCGCGTCGAAGCCGAGGATGCAGCCACATACTTTT
GCCACCAGTGGTCCTCTAATCCCCTGACATTTGGCGCAGGCACCAAGCTCGAACTGAAGCGGGGATCTACCAGCGGCAGTGGAAAACC
TGGTAGCGGAGAGGGGTCTACCAAGGGGCAAGTGCAACTCAGACAGCCTGGCGCAGAACTCGTGAAGCCCGGCGCATCCGTCAAGATG
TCTTGTAAAGCCAGTGGCTACACCTTCACTAGCTATAATATGCACTGGGTTAAACAGACTCCCGGTCAGGGCCTCGAGTGGATTGGCG
CAATCTACCCGGGTAATGGTGATACTTCTTACAATCAGAAGTTTAAGGGGAAAGCTACCCTGACCGCCGACAAGTCTTCATCAACCGC
CTACATGCAGCTGAGCTCTCTGACGTCTGAGGATTCCGCCGTGTATTACTGCGCCCGGAGCCACTACGGGAGTAATTATGTCGATTAT
TTTGACTACTGGGGTCAGGGAACAACCCTGACTGTCTCCAGCTCCGGAACCACGACGCCAGCGCCGCGACCACCAACACCGGCGCCCA
CCATCGCGTCGCAGCCCCTGTCCCTGCGCCCAGAGGCGTGCCGGCCAGCGGCGGGGGGCGCAGTGCACACGAGGGGGCTGGACTTCGC
CTGTGATATCTACATCTGGGCGCCCTTGGCCGGGACTTGTGGGGTCCTTCTCCTGTCACTGGTTATCACCCTTTACTGCAAACGGGGC
AGAAAGAAACTCCTGTATATATTCAAACAACCATTTATGAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGCGCAGACGCCCCCGCGTACAAGCAGGGCCAGAACCAGCT
CTATAACGAGCTCAATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAGGACACCTACGACGCCCTTCACATGCAGGC
CCTGCCCCCTCGCTAAG

Amino acid sequences
1. Anti-human IL-6 receptor single-chain
variable fragment crystallizable region (anti-
hIL-6R scFv-Fc)
(SEQ ID NO: 22)

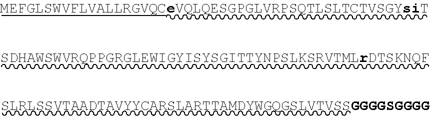

SGGGGSDIQMTQSPSSLSASVGDRVTITCRASQDISSYLNWYQQQKPGK
APKLLIYYTSRLHSGVPSRFSGSGSGTDFTFTISSLQPEDIATYYCQQG
NTLPYTFGQGTKVEIKVEPKSSDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPGKGSEQKLISEEDLrakr
Signal peptide derived from IgG variable heavy
chain: underline
Variable heavy chain: wavy underline
Linker: bold
Variable light chain: double underline
IgG1 Fc with C103S mutation: bold and underline
Myc tag: bold italics
Furin cleavage site to enable linking multiple
effectors via 2A peptide: italics
*What is not in capital but bold is different
from the patent that we are referring to in the
text (4 amino acid difference). This sequence was
derived from combining sequence of
tocilizumab from this patent and this article:
Physicochemical and Biological
Characterization of the Proposed Biosimilar
Tocilizumab. Shiwei Miao,1 Li Fan,1 Liang
Zhao,1 Ding Ding,2 Xiaohui Liu,2 Haibin Wang,2
and Wen-Song Tan1 1State Key
Laboratory of Bioreactor Engineering, East China
University of Science and Technology,
Shanghai 200237, China 2Hisun Pharmaceutical
(Hangzhou) Co., Ltd, Fuyang,
Hangzhou, Zhejiang 311404, China
2. Anti-CD20 CAR human (hCD8 leader-CD20 scFv-
hCD8 hinge-hCD8 transmembrane-
h4-1BB-hCD3 zeta); h refers to human sequence
(SEQ ID NO: 23)
MALPVTALLLPLALLLHAARPGSDIVLSQSPAILSASPGEKVTMTCRAS
SSLSFMHWYQQKPGSSPKPWIYATSNLASGVPARFSGSGSGTSYSLTIS
RVEAEDAATYFCHQWSSNPLTFGAGTKLELKRGSTSGSGKPGSGEGSTK
GQVQLRQPGAELVKPGASVKMSCKASGYTFTSYNMEIWVKQTPGQGLEW
IGAIYPGNGDTSYNQKFKGKATLTADKSSSTAYMQLSSLTSEDSAVYYC
ARSHYGSNYVDYFDYWGQGTTLTVSSSGTTTPAPRPPTPAPTIASQPLS
LRPEACRPAAGGAVHTRGLDFACDIYIWAPLAGTCGVLLLSLVITLYCK
RGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR
3. Anti-CD20 CAR canine (cCD8 leader-CD20 scFv-
cCD8 hinge-cCD8 transmembrane-c4-
1BB-cCD3 zeta); c refers to canine sequence
(SEQ ID NO: 24)
MASRVTALLLPLALLLRAAAASDIVLSQSPAILSASPGEKVTMTCRASS
SLSFMHWYQQKPGSSPKPWIYATSNLASGVPARFSGSGSGTSYSLTISR
VEAEDAATYFCHQWSSNPLTFGAGTKLELKRGSTSGSGKPGSGEGSTKG
QVQLRQPGAELVKPGASVKMSCKASGYTFTSYNMEIWVKQTPGQGLEWI
GAIYPGNGDTSYNQKFKGKATLTADKSSSTAYMQLSSLTSEDSAVYYCA
RSHYGSNYVDYFDYWGQGTTLTVSSASPTTPAPRPPTRAPTNASKPVSP
RGETCRPAAGSAVKTSGLDFACEIYIWAPLAGTCAVLLLSLVITIICHG
RKKLLYLFKQPFMRPVQTAQEEDACSCRFPEEEEGECDLRAKFGRSAAA
PEHQQGPNQLYNELNLRGREEYEVLDKRRGLDPEMGGKQRKRNPQEVVY
NALQKDKMAEAYSEIGIKSENQRRRGKGHDGLYQGLSTATKDTYDALHM
QALPPR
4. Anti-Her2 human CAR with 4-1BB costimulatory
domain (hCD8 leader-4D5 scFv-hCD8
hinge-hCD8 transmembrane-h4-1BB-hCD3 zeta);
h refers to human sequence
(SEQ ID NO: 25)
MALPVTALLLPLALLLHAARPDIQMTQSPSSLSASVGDRVTITCRASQD
VNTAVAWYQQKPGKAPKLLIYSASFLYSGVPSRFSGSRSGTDFTLTISS
LQPEDFATYYCQQHYTTPPTFGQGTKVEIKRTGSTSGSGKPGSGEGSEV
QLVESGGGLVQPGGSLRLSCAASGFNIKDTYIEWVRQAPGKGLEWVART
YPTNGYTRYADSVKGRFTISADTSKNTAYLQMNSLRAEDTAVYYCSRWG
GDGFYAMDVWGQGTLVTVSSSGTTTPAPRPPTPAPTIASQPLSLRPEAC
RPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
5. Anti-Her2 human CAR with CD28 costimulatory
domain (hCD8 leader-4D5 scFv-hCD8
hinge-hCD8 transmembrane-hCD28-hCD3 zeta);
h refers to human sequence
(SEQ ID NO: 26)
MALPVTALLLPLALLLHAARPDIQMTQSPSSLSASVGDRVTITCRASQD
VNTAVAWYQQKPGKAPKLLIYSASFLYSGVPSRFSGSRSGTDFTLTISS
LQPEDFATYYCQQHYTTPPTFGQGTKVEIKRTGSTSGSGKPGSGEGSEV
QLVESGGGLVQPGGSLRLSCAASGFNIKDTYIHWVRQAPGKGLEWVART
YPTNGYTRYADSVKGRFTISADTSKNTAYLQMNSLRAEDTAVYYCSRWG
GDGFYAMDVWGQGTLVTVSSSGTTTPAPRPPTPAPTIASQPLSLRPEAC
RPAAGGAVHTRGLDFACDFWVLVVVGGVLACYSLLVTVAFIIFWVRSKR
SRLLHSDYMNMTPRRPGPTRKHYQPYAPPRDFAAYRSIDRVKFSRSADA
PAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQA
LPPR
6. hIL-12 (p40-linker-p35); h refers to human
(SEQ ID NO: 27)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSGGGGGGSRNLPVATP
DPGMFPCLHEISQNLLRAVSNMLQKARQTLEFYPCTSEEIDHEDITKDK
TSTVEACLPLELTKNESCLNSRETSFITNGSCLASRKTSFMMALCLSSI
YEDLKMYQVEFKTMNAKLLMDPKRQIFLDQNMLAVIDELMQALNFNSET
VPQKSSLEEPDFYKTKIKLCILLHAFRIRAVTIDRVMSYLNAS
7. cIL-12 (p40-1inker-p35); c refers to canine
(SEQ ID NO: 28)
MHPQQLVISWFSLVLLASSLMTIWELEKDVYVVELDWHPDAPGEMVVLT
CHTPEEDDITWTSAQSSEVLGSGKTLTIQVKEFGDAGQYTCHKGGKVLS
RSLLLIHKKEDGIWSTDILKEQKESKNKIFLKCEAKNYSGRFTCWWLTA
ISTDLKFSVKSSRGFSDPQGVTCGAVTLSAERVRVDNRDYKKYTVECQE
GSACPSAEESLPIEVVVDAIHKLKYENYTSSFFIRDIIKPDPPTNLQLK
PLKNSRHVEVSWEYPDTWSTPHSYFSLTFCVQAQGKNNREKKDRLCVDK
TSAKVVCHKDAKIRVQARDRYYSSSWSDWASVSCSGGGGGGSRSLPTAS
PSPGIFQCLNHSQNLLRAVSNTLQKARQTLELYSCTSEEIDHEDITKDK
TSTVEACLPLELTMNESCLASREISLITNGSCLASGKASFMTVLCLSSI
YEDLKMYQMEFKAMNAKLLMDPKRQIFLDQNMLTAIDELLQALNFNSVT
VPQKSSLEEPDFYKTKIKLCILLHAFRIRAVTIDRMMSYLNSS

### Example 13—Effect of ATG Sequence Between the Minimal Promoter and the Spacer

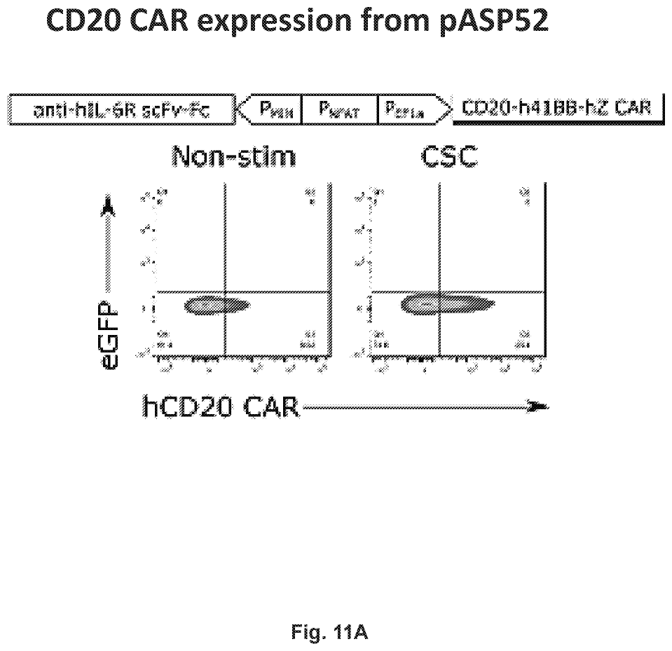

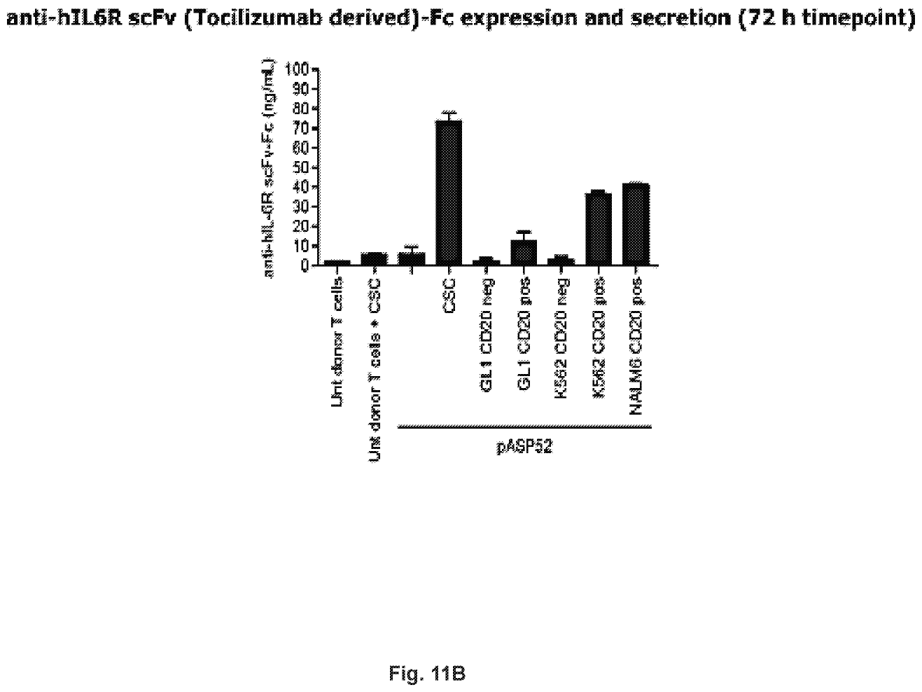

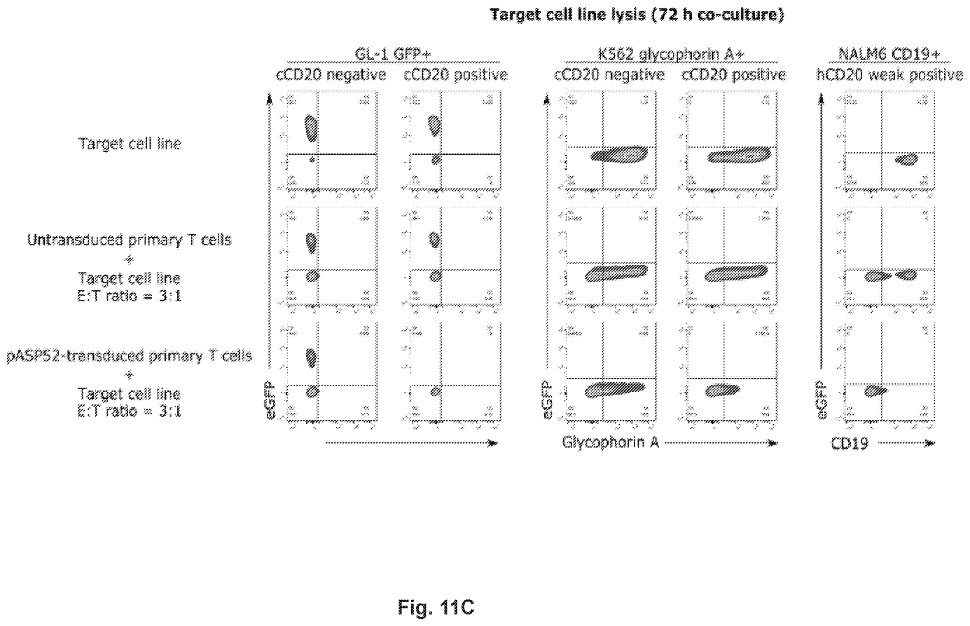

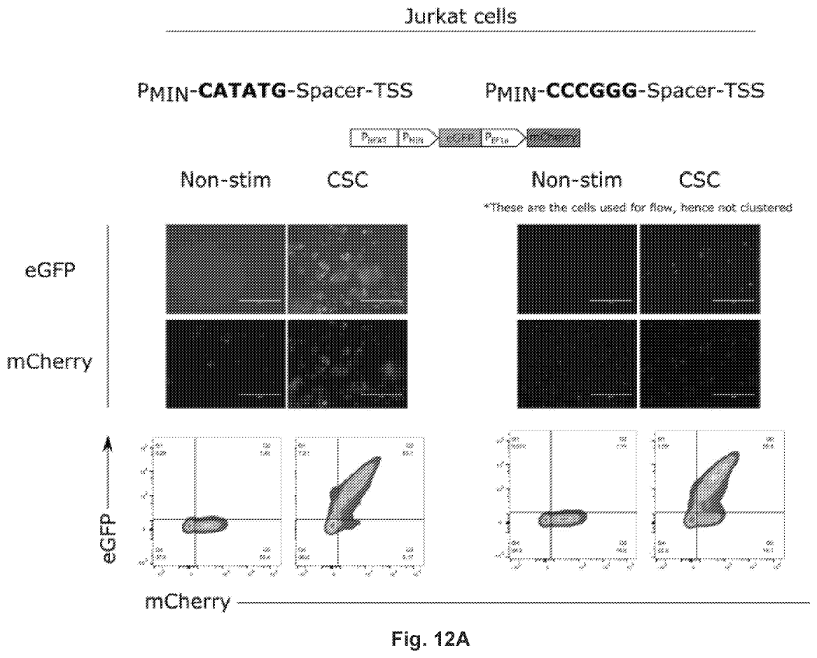

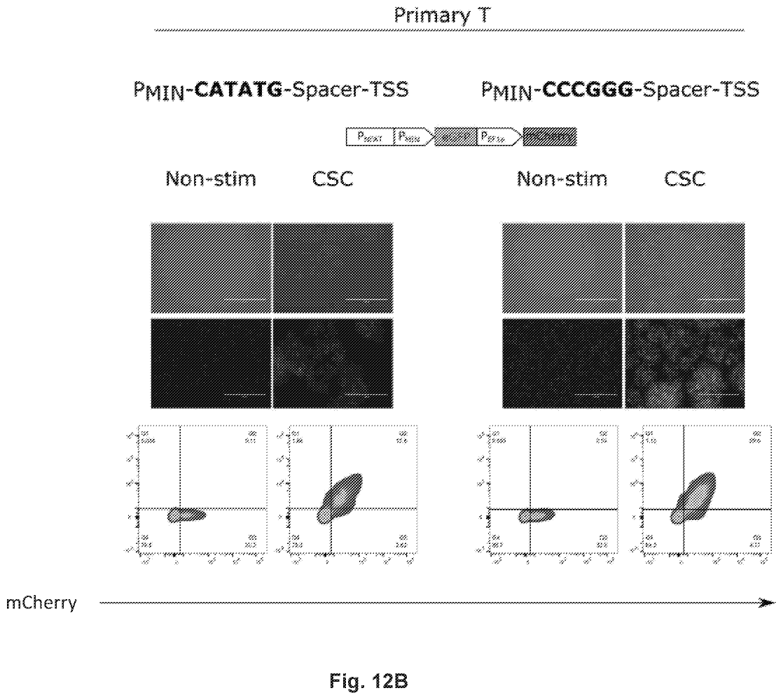

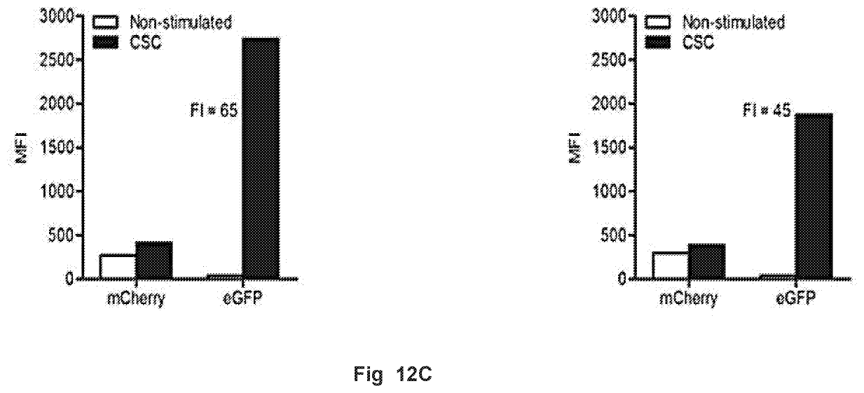

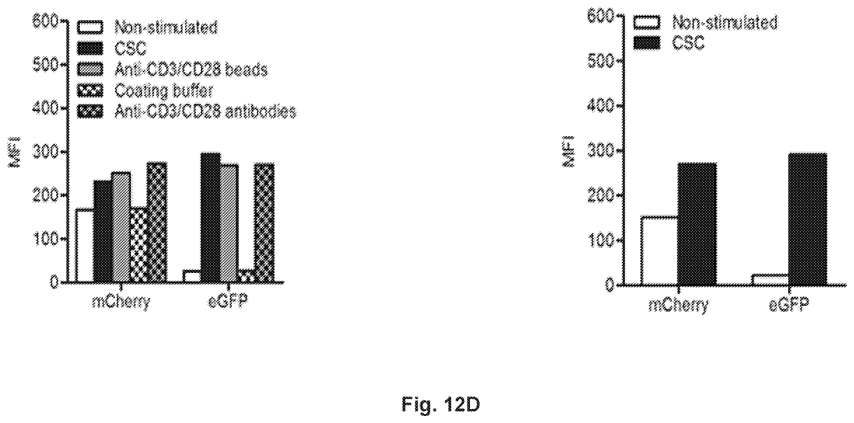

The effect of 6-nucleotide sequences placed between the minimal promoter and the spacer in the inducible module was investigated. How the presence of ATG as a potential upstream start codon influences the performance of the system was studied. It was demonstrated herein that a CATATG 6-nucleotide sequence (SEQ ID NO: 1) and a CCCGGG 6-nucleotide sequence (SEQ ID NO: 2) perform similarly in terms of transduction efficacy, leakage and maximal activity of the system (FIGS. 12A-12D). Thus, a potential ATG start codon was found not to interfere with translation from the intended and optimized start codon with an optimal Kozak sequence. Without wishing to be bound by theory, the position of this ATG is likely too close downstream to the TATA box (21 nt) in the minimal promoter and hence was not transcribed and present in the mRNA.

The sequence CCCGGG (SEQ ID NO: 2), without an upstream ATG sequence between the minimal promoter and the spacer, may be found in contructs pASP52 (anti-hIL-6R scFv-Fc reverse+CD20-targeting CAR forward), pASP72 (TCF7 reverse+Her2-targeting CAR forward), pASP73 (FOXO1-3A reverse+Her2-targeting CAR forward), pASP79 (c225-OKT3 BiTE reverse+IL-13Ra2-targeting CAR forward), pASP83 (806-OKT3 BiTE reverse+IL-13Ra2-targeting CAR forward), lentiviral vectors from 12A and 12B (eGFP forward-mCherry forward; I didn't name them, but we can maybe say pASP4.2.1, since it is variation of pASP4.2) where we investigate functionality without this non-intentional upstream ATG.

Any DNA sequence that modulates distance between inducible and constitutive part when in the back-to-back orientation may be integrated. In some embodiments, DNA sequences without known function and without unfavorable secondary structures are used. In some embodiments, a spacer used for cloning purposes may be present.

The following sequence, or any amount of repetitions thereof, may be used as a spacer:

(SEQ ID NO: 29)
5′-GGCATTCCGGTACTGTTGGTAAA- 3′

### Example 14—CAR Downregulation and Re-Expression on Cell Surface

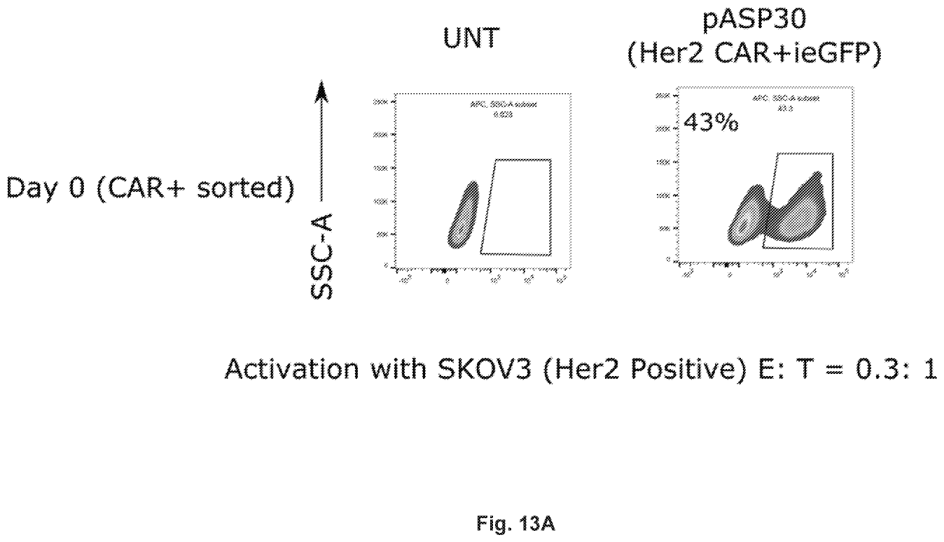

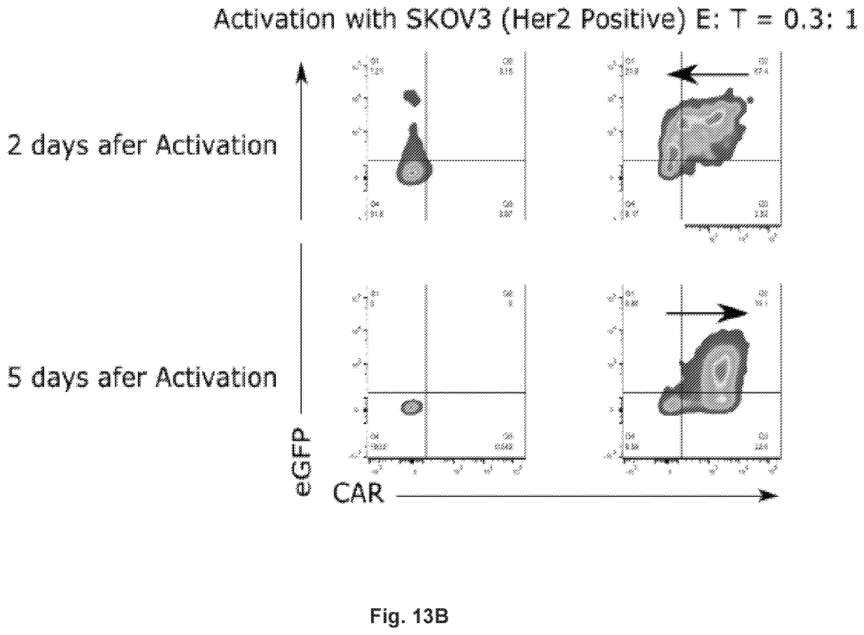

The surface downregulation of CAR after ligating cognate antigen on target cells, which was observed in FIGS. 7A-7B, was further characterized. Primary T cells were activated, transduced with a single-vector system pASP30 (inducible eGFP+constitutive 4D5 anti-Her2 scFv 4-1BB+CD3-zeta CAR) at MOI5, expanded and rested according to the standard protocol. At the end of the expansion they were sorted for CAR+ cell population (Day 0). Cells were then co-cultured with SKOV3 cancer cell line that expresses high levels of Her2 at 0.3:1 effector to target ratio. Two days later cell were stained for CAR expression and profound downregulation of CAR was observed in cells that recognized target antigen as evidenced by upregulated NFAT inducible eGFP module in this population. Five days after the co-culture was set up, CAR T cells lysed a large majority of SKOV3 target cells which led to the re-expression of CAR. This demonstrated target mediated downregulation and re-expression of CAR on the cell surface (FIGS. 13A-13B).

Thus, by starting with a sorted CAR positive population, it was demonstrated that the CAR is downregulated on the cell surface after binding antigen-positive cells, but because of its constitutive expression, it is re-expressed after the target cells are lysed. (FIGS. 13A-13B). Additionally, in this way, CAR T cells that normally downregulate surface CAR expression after activation may now be detected via induced intracellular GFP expression.

### Example 15—In Vivo Validation of the System

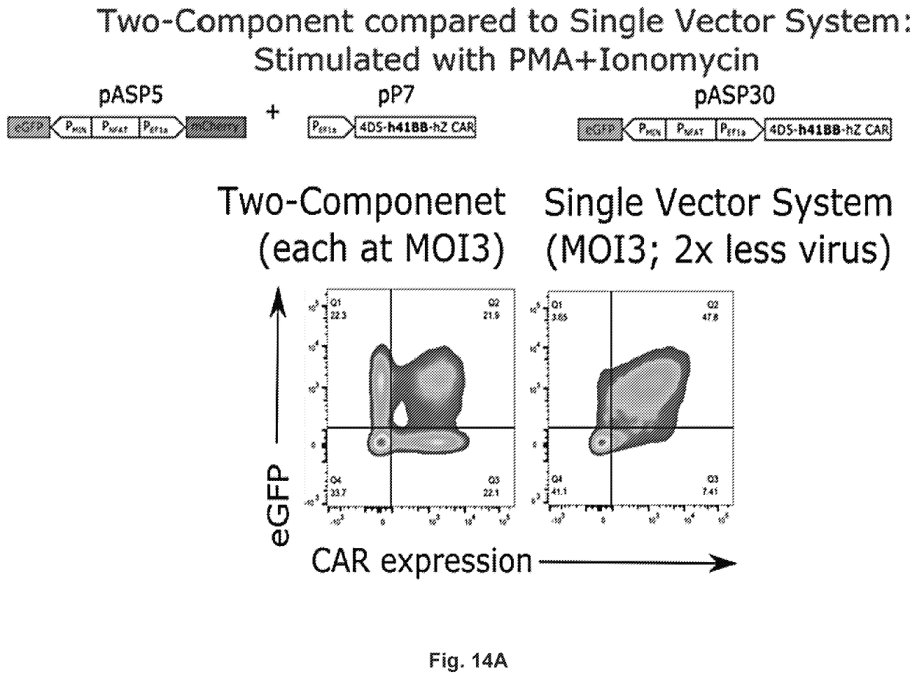

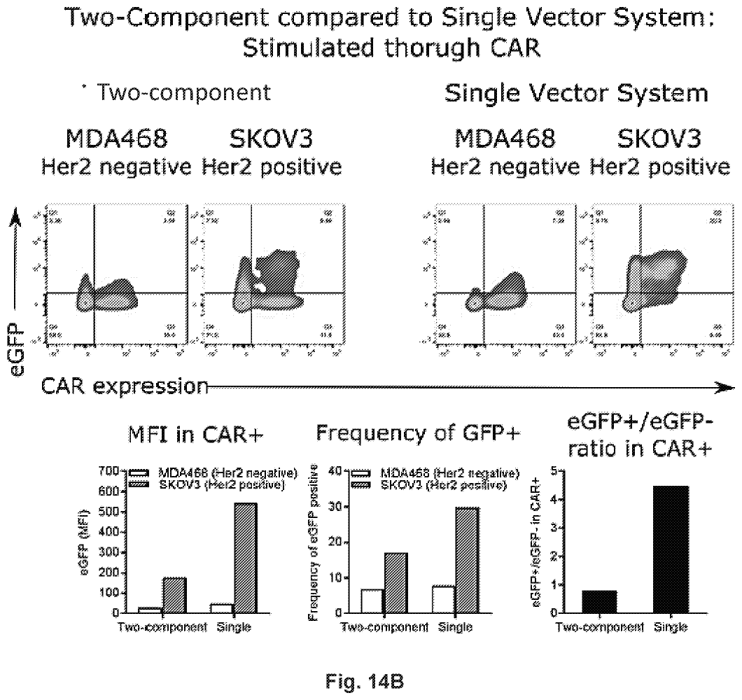

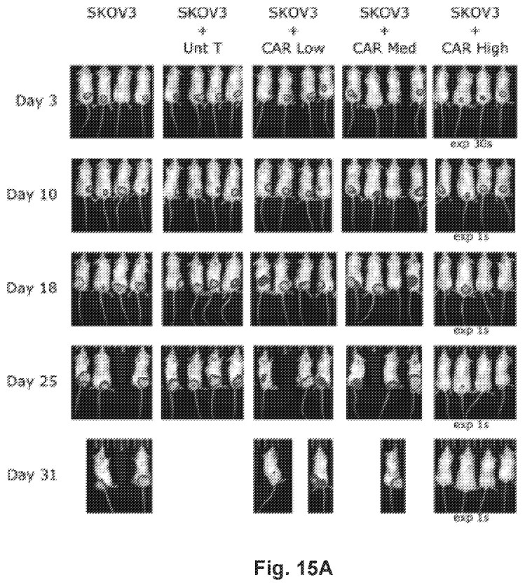

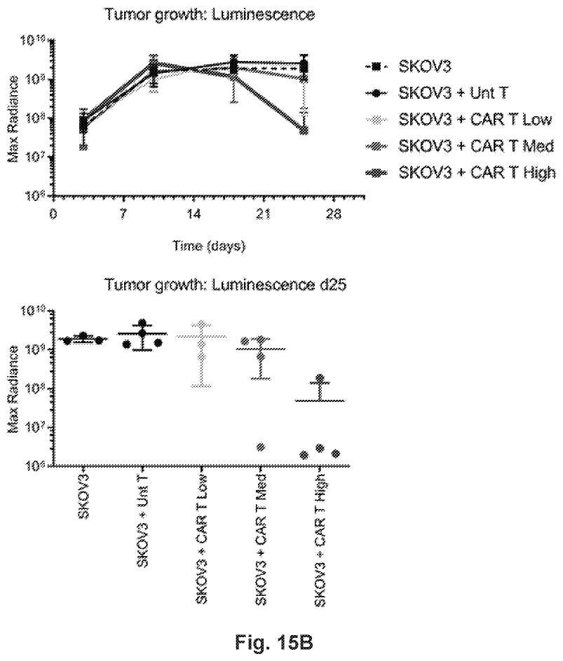

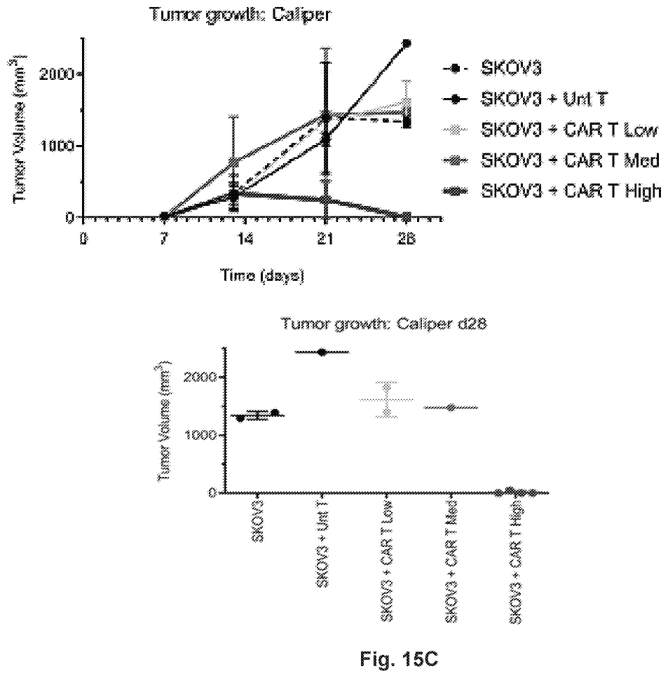

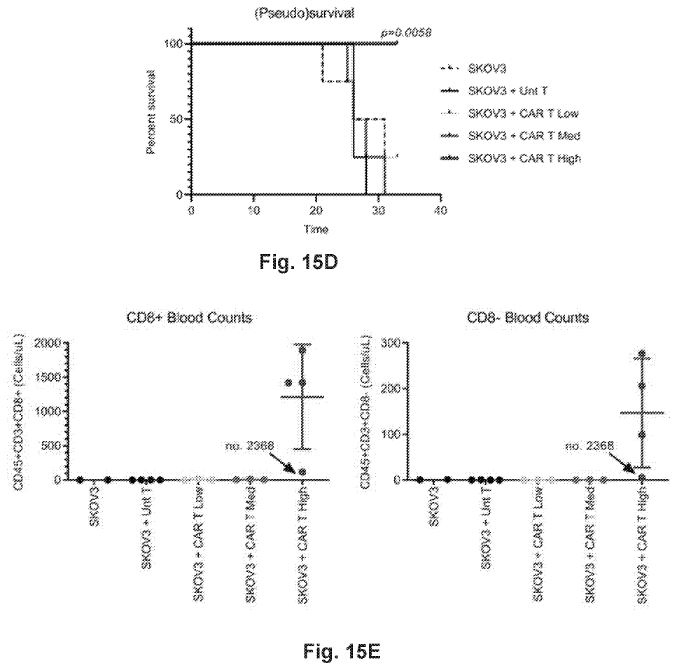

The in vivo functionality of the system was shown by testing an example of the single-vector system (pASP26; inducible eGFP+constitutive 4D5 anti-Her2 scFv 4-1BB+CD3-zeta CAR; forward orientation) in a tumor xenograft model. One million CAR+ primary T cells controlled established SKOV3 tumor growth as evidenced by no detectible tumor measured by imaging (FIG. 15A), quantification of luciferase activity (FIG. 15B), caliper measurement of tumor growth (FIG. 15C) and survival (FIG. 15D). Blood counts of human primary T cells in mice correlated with tumor control where persistence was only observed in the highest dose of CAR+ T cells (FIG. 15E). Although a dose of 1 million CAR+ primary T cells per mice is depicted here as the group “High”, this is actually low dose of CAR T cells compared to other studies. This data shows that CAR T cells with activation induced transgene (GFP) expression have the capacity to mediate tumor control in vivo.

### Example 16—CAR T Cells Designed to Secrete an Activation Inducible Anti-hIL-6R scFv-Fc Secrete Biologically Active scFv-Fc after Antigen-Specific CAR T Cell Stimulation as a Method to Reduce Cytokine Release Syndrome

To demonstrate functionality of autonomously secreted engineered human IL-6Ra-blocking antibody from primary T cells, its binding capacity to human IL-6Ra expressed on the cell surface was investigated. Primary T cells were activated, transduced with pASP52 (inducible anti-hIL-6R-Myc tag+constitutive anti-CD20 scFv 4-1BB+CD3-zeta CAR; back-to-back orientation) or control pP2 construct (constitutive anti-CD20 scFv 4-1BB+CD3-zeta CAR) at MOI5, expanded and rested according to the standard protocol. Engineered T cells were co-cultured with CD20 positive cell line NALM6 at 3:1 ratio for 72 h to induce antigen-dependent expression and secretion of hIL-6R blocking antibody. Supernatant from these co-cultures was added to the HEK 293T cells engineered to express hIL-6Ra or control parental cells (with no detectable IL-6Ra expression) and incubated for 1 h. Binding anti-hIL-6R scFv-Fc-Myc tag was detected by secondary stain against Myc tag. Only supernatant from anti-hIL-6R scFv-Fc producing, but not control engineered T cells stained target IL-6Ra expressing cells in a dose dependent manner. The same supernatant did not stain parental HEK 293 T cells demonstrating specific binding. Commercial recombinant anti-hIL-6R antibody was used as a positive control. SN means supernatant.

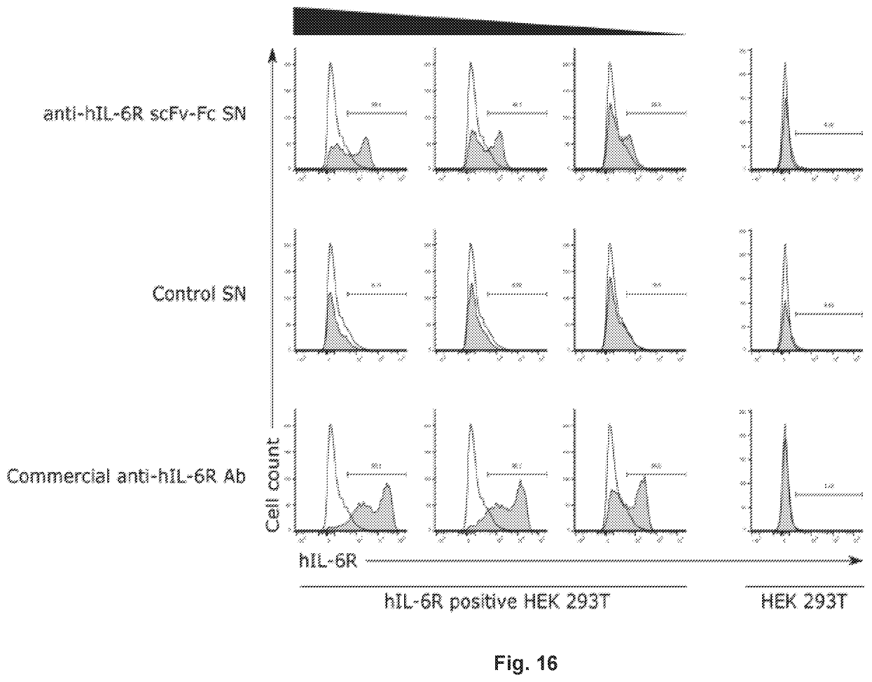

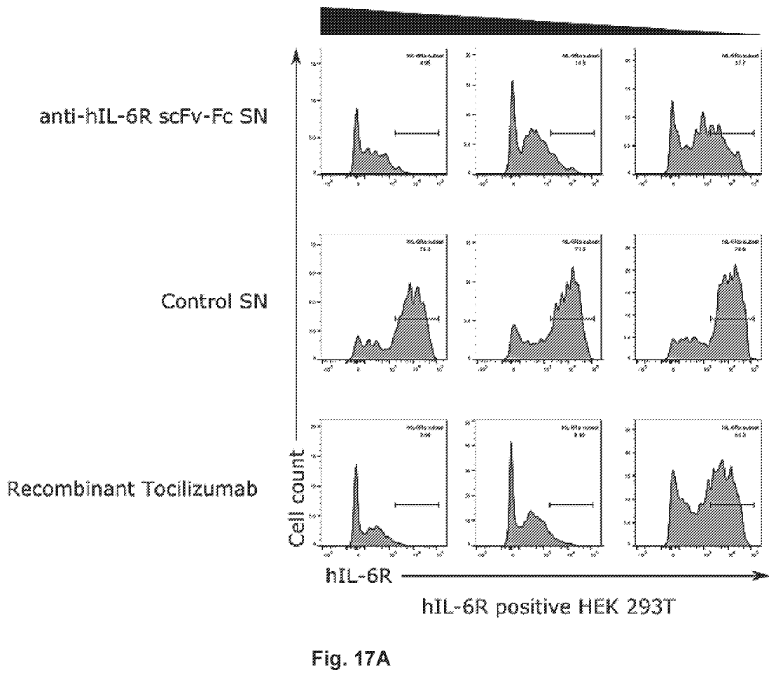

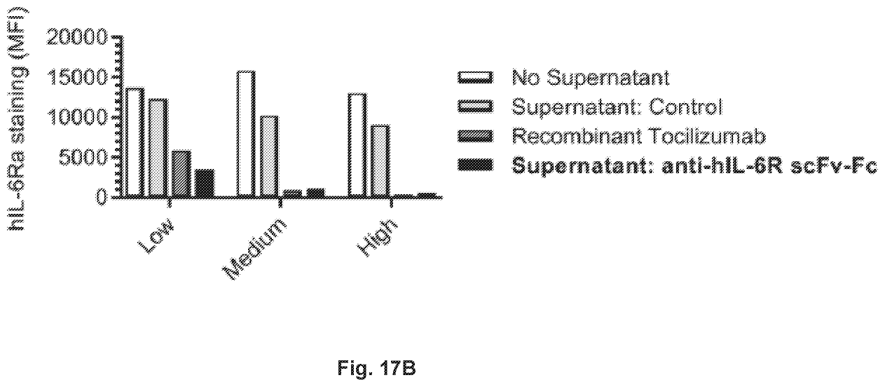

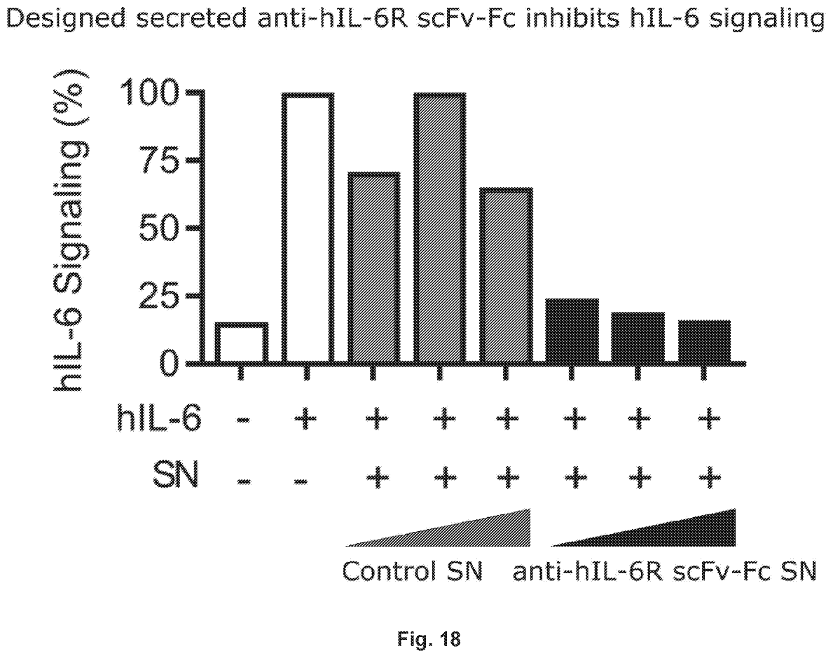

Only supernatant from anti-hIL-6R scFv-Fc producing, but not control engineered T cells (both activated specifically though CAR), stained IL-6Ra-expressing cells in a dose dependent manner. The same supernatant did not stain parental HEK 293 T cells that do not express human IL-6Ra, demonstrating specificity of the binding (FIG. 16).

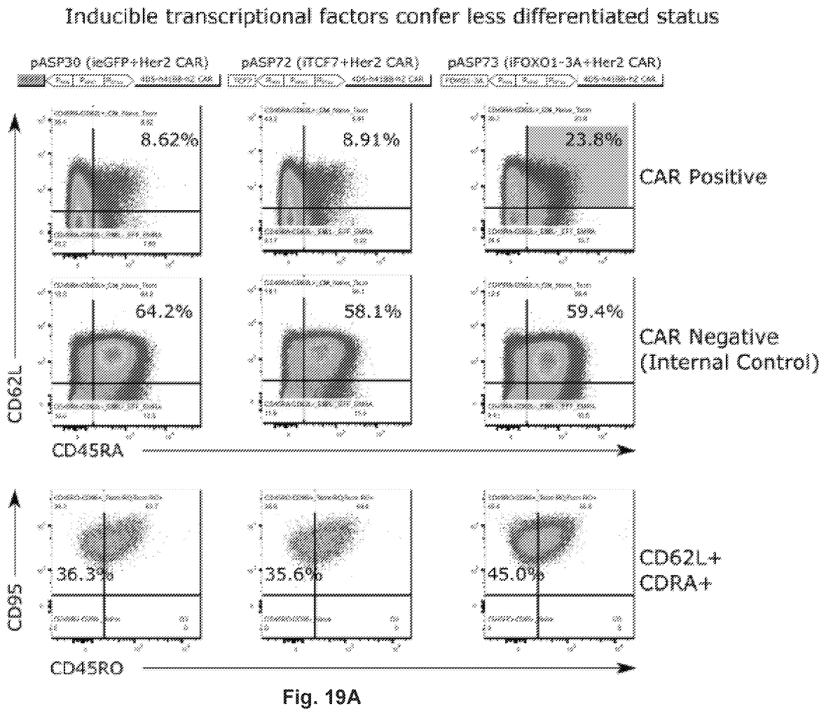

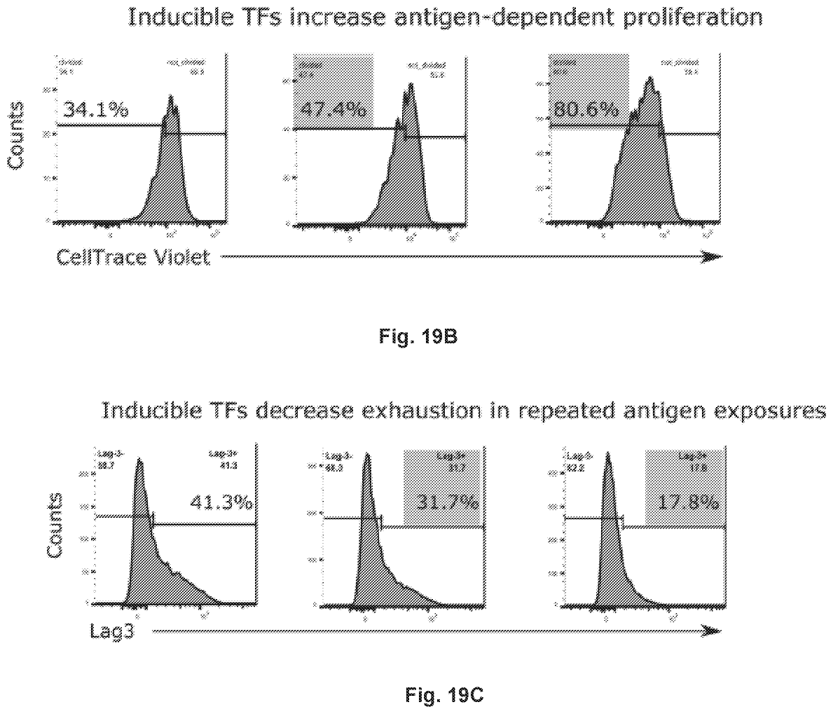

To further demonstrate biological activity of secreted engineered human IL-6Ra-blocking antibody, whether its binding to human IL-6Ra expressed on the cell surface would block subsequent binding of commercial anti-hIL-6Ra antibody was investigated. Only supernatant from anti-hIL-6R scFv-Fc producing HEK 293T cells, but not control cells secreting irrelevant antibody, blocked staining of HEK 293T cells engineered to express hIL-6Ra with recombinant anti-IL-6Ra antibody. The blocking effect was found to be dose-dependent. This demonstrated that as designed, the engineered anti-hIL-6R scFv-Fc specifically interacts with hIL-6Ra and has capacity to block binding of other molecules to human IL-6Ra. See FIGS. 19A-19B.

### Example 17—Antigen-Inducible Transcriptional Factors TCF-7 and FOXO1-3A Mediate Less Differentiated Status and Improved Antigen-Dependent Proliferation in CAR T Cells

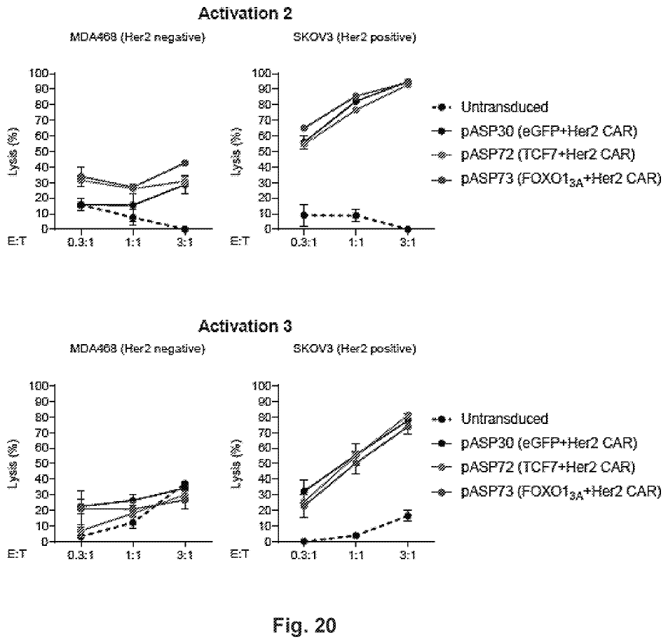

Emerging concepts show that particular phenotypes of engineered immune cells, such as memory component and less differentiated status, are beneficial for therapeutic effect in cell based immunotherapies. By introducing transcriptional factors that are known to regulate memory phenotype and stemness as an activation inducible module in the single-vector system, CAR T cell products with a less differentiated phenotype and improved function were developed. As an example, NFAT-inducible TCF-7 and FOXO1 transcriptional factors were implemented in a combination with a Her2-targeting CAR. Since T cell stimulation activates signaling pathways that phosphorylate human wild type FOXO1 which leads to its nuclear export and inactivation of transcriptional activity, key residues were mutated (T24-A24, 5256-A256 and S319-A319) according to the published work of Kodama et al. Mol Cell Biol. 2004 September; 24(18):7931-40, and according to the human FOXO1 sequence in UniProt (www.uniprot.org/uniprot/Q12778). With this intervention, a FOXO1-3A that is resistant to AKT mediated phosphorylation and is hence insensitive to nuclear export was generated. In CAR T cells exposed to repeated stimulations with target cancer cells, FOXO1-3A was shown to preserve a less differentiated status as determined by increased frequency of CD62L positive, Naïve (CD62L⁺, CD45RA⁺, CD95⁻) and stem memory T cell T_{SCM}(CD62L⁺, CD45RA⁺, CD95⁺) populations compared to control cells that express inducible eGFP (FIG. 19A). Inducible expression of both TCF-7 and FOXO1-3A increased antigen-dependent proliferation of CAR T cells upon repeated stimulations, demonstrating improved functionalities instilled by the single-vector system (FIG. 19B). Antigen-independent proliferation was not observed. Finally, decreased expression of exhaustion marker Lag3 was observed in TCF-7 and FOXO1-3A engineered CAR T cells compared to eGFP control (FIG. 19C). At the same time, incorporation of the inducible TCF-7 and FOXO1-3A did not impair lysis capacity of the engineered Her2-targeting CAR T cells even after several rounds of antigen exposure (FIG. 20).

Together, this data suggests that antigen-dependent expression of transcriptional factors that modify epigenetic profile and phenotype of CAR T cells can improve CAR T cell functions when integrated in the single-vector system. Importantly, the single-vector system ensures that the genetic information for a CAR and transcriptional factor is delivered together, which enables coordinated expression of transcriptional factor induced by CAR signaling in the same T cell. This is in contrast with two-component vector systems where only 30% of the transduced population integrates both constitutive and inducible modules, as demonstrated in FIGS. 14A-14B.

Amino Acid Sequence of TCF-7 (SEQ ID NO: 30):
MPQLDSGGGGAGGGDDLGAPDELLAFQDEGEEQDDKSRDSAAGPERDLAE
LKSSLVNESEGAAGGAGIPGVPGAGAGARGEAEALGREHAAQRLFPDKLP
EPLEDGLKAPECTSGMYKETVYSAFNLLMHYPPPSGAGQHPQPQPPLHKA
NQPPHGVPQLSLYEHFNSPHPTPAPADISQKQVHRPLQTPDLSGFYSLTS
GSMGQLPHTVSWFTHPSLMLGSGVPGHPAAIPHPAIVPPSGKQELQPFDR
NLKTQAESKAEKEAKKPTIKKPLNAFMLYMKEMRAKVIAECTLKESAAIN
QILGRRWHALSREEQAKYYELARKERQLHMQLYPGWSARDNYGKKKRRSR
EKHQESTTETNWPRELKDGNGQESLSMSSSSSPA
Amino Acid Sequence of FOXO1-3A (SEQ ID NO: 31):
MAEAPQVVEIDPDFEPLPRPRSCAWPLPRPEFSQSNSATSSPAPSGSAAA
NPDAAAGLPSASAAAVSADFMSNLSLLEESEDFPQAPGSVAAAVAAAAAA
AATGGLCGDFQGPEAGCLHPAPPQPPPPGPLSQHPPVPPAAAGPLAGQPR
KSSSSRRNAWGNLSYADLITKAIESSAEKRLTLSQIYEWMVKSVPYFKDK
GDSNSSAGWKNSIRHNLSLHSKFIRVQNEGTGKSSWWMLNPEGGKSGKSP
RRRAAAMDNNSKFAKSRSRAAKKKASLQSGQEGAGDSPGSQFSKWPASPG
SHSNDDFDNWSTFRPRTSANASTISGRLSPIIVITEQDDLGEGDVHSMVY
PPSAAKMASTLPSLSEISNPENMENLLDNLNLLSSPTSLTVSTQSSPGTM
MQQTPCYSFAPPNTSLNSPSPNYQKYTYGQSSMSPLPQMPIQTLQDNKSS
YGGMSQYNCAPGLLKELLTSDSPPHNDIMTPVDPGVAQPNSRVLGQNVMM
GPNSVMSTYGSQASHNKMMNPSSHTHPGHAQQTSAVNGRPLPHTVSTMPH
TSGMNRLTQVKTPVQVPLPHPMQMSALGGYSSVSSCNGYGRMGLLHQEKL
PSDLDGMFIERLDCDMESIIRNDLMDGDTLDFNFDNVLPNQSFPHSVKTT
THSWVSG

### Example 18—Adaptation of the Single-Vector System to Sense and Respond to IL-6

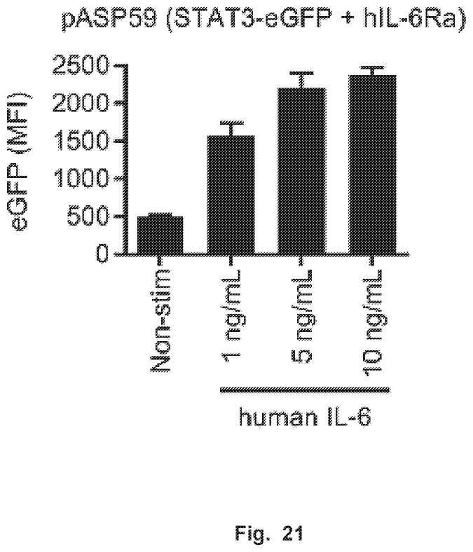

The single-vector system is modularly designed which makes it amenable for customized design in regards to selection of the immune receptor (here we show use of Her2, CD20 and IL-13Ra2 targeting CARs) and inducible effectors (here we show fluorescent proteins, IL-12, IL-6Ra blocking antibody, TCF-7 and FOXO1-3A transcriptional factors and bispecific T cell engagers). Additionally, by exchanging the DNA response element (promoter region), this system can be engineered to sense customized inputs. As an example, NFAT-sensing promoter was exchanged with the STAT3-sensing promoter and the CAR was exchanged with IL-6Ra in the single-vector system to generate pASP59 (STAT3-eGFP+hIL-6Ra). HEK 293 T cells were transduced with pASP59 and stimulated with increasing amounts of human IL-6. Specific and dose dependent response was observed, demonstrating modular design of the single-vector system where elements (receptors and promoters) can be easily exchanged to detect customized signals and respond by a relevant therapeutic output (FIG. 21). IL-6 sense-response system is useful to generate sensor cell lines to monitor IL-6 signaling pathways, for example detecting IL-6 in the samples or to monitor inhibition of IL-6 by IL-6 blocking antibodies. Therapeutically, sensing IL-6 and responding by producing IL-6 blocking antibody by T cells or other engineered cells in the context of cytokine release syndrome is an alternative to the NFAT-responding system.

Sequence of STAT3 responsive element
(SEQ ID NO: 32):
CCGGTACTGTTAGCTTCATTTCCCGTAAATCGTCGAAGCTTCATTTCCC
GTAAATCGTCGAAGCTTCATTTCCCGTAAATCGTCGAAGCTTCATTTCC
CGTAAATCGTCGA
Sequence of hIL-6Ra (SEQ ID NO: 33):
ATGCTGGCCGTGGGATGTGCTTTGCTTGCTGCTCTGTTGGCTGCTCCTG
GTGCTGCACTGGCCCCTAGAAGATGTCCTGCTCAAGAGGTGGCAAGAGG
CGTGCTGACATCTCTGCCTGGCGATAGCGTGACCCTGACATGTCCTGGC
GTGGAACCCGAGGATAATGCCACCGTGCATTGGGTGCTGAGAAAGCCTG
CCGCCGGATCTCATCCTAGCAGATGGGCTGGCATGGGCCGTAGACTGCT
GCTGAGATCTGTCCAGCTGCACGACAGCGGCAACTACAGCTGTTACAGA
GCCGGCAGACCTGCCGGAACAGTGCATCTGCTGGTGGATGTGCCTCCTG
AGGAACCCCAGCTGAGCTGCTTCAGAAAGTCCCCTCTGAGCAACGTCGT
GTGCGAGTGGGGCCCTAGAAGCACACCTAGCCTGACAACAAAGGCCGTG
CTGCTCGTGCGGAAGTTCCAGAATTCTCCCGCCGAGGACTTCCAAGAGC
CTTGCCAGTACAGCCAAGAGAGCCAGAAGTTCAGCTGTCAGCTGGCTGT
GCCTGAGGGCGACAGCAGCTTCTACATCGTGTCTATGTGCGTGGCCAGC
AGCGTGGGCAGCAAGTTTAGCAAGACCCAGACCTTCCAAGGCTGCGGCA
TCCTGCAACCTGATCCTCCAGCCAACATCACCGTGACCGCCGTGGCCAG
AAATCCCAGATGGCTGTCTGTGACCTGGCAGGACCCTCACAGCTGGAAC
TCCAGCTTTTACCGGCTGAGATTCGAGCTGCGGTACAGGGCCGAGAGAA
GCAAGACCTTCACCACCTGGATGGTCAAGGACCTGCAGCATCACTGCGT
GATCCACGATGCTTGGAGCGGCCTGAGACATGTGGTGCAGCTGAGAGCC
CAAGAGGAATTCGGCCAAGGCGAGTGGAGTGAATGGTCCCCTGAAGCCA
TGGGCACCCCTTGGACAGAGAGCAGATCTCCACCAGCCGAGAACGAGGT
GTCCACACCTATGCAGGCCCTGACCACCAACAAGGACGACGACAACATC
CTGTTCCGGGACAGCGCCAATGCCACAAGTCTGCCTGTGCAGGATAGCT
CCTCTGTGCCCCTGCCTACCTTTCTGGTTGCTGGCGGATCTCTGGCCTT
TGGCACCCTGCTGTGTATCGCCATCGTGCTGCGGTTCAAGAAAACCTGG
AAGCTGCGGGCCCTGAAAGAGGGCAAGACCTCTATGCACCCTCCTTACA
GCCTGGGCCAGCTGGTGCCTGAAAGACCTAGACCTACACCTGTGCTGGT
GCCCCTGATCAGCCCTCCTGTGTCTCCATCTAGCCTGGGCAGCGACAAT
ACCAGCAGCCACAACAGACCCGACGCTCGGGACCCTAGATCTCCCTACG
ACATCAGCAATACCGACTACTTCTTCCCACGC

### Example 19—Use of the Single-Vector System to Screen for Antigen-Specific Immune Cells

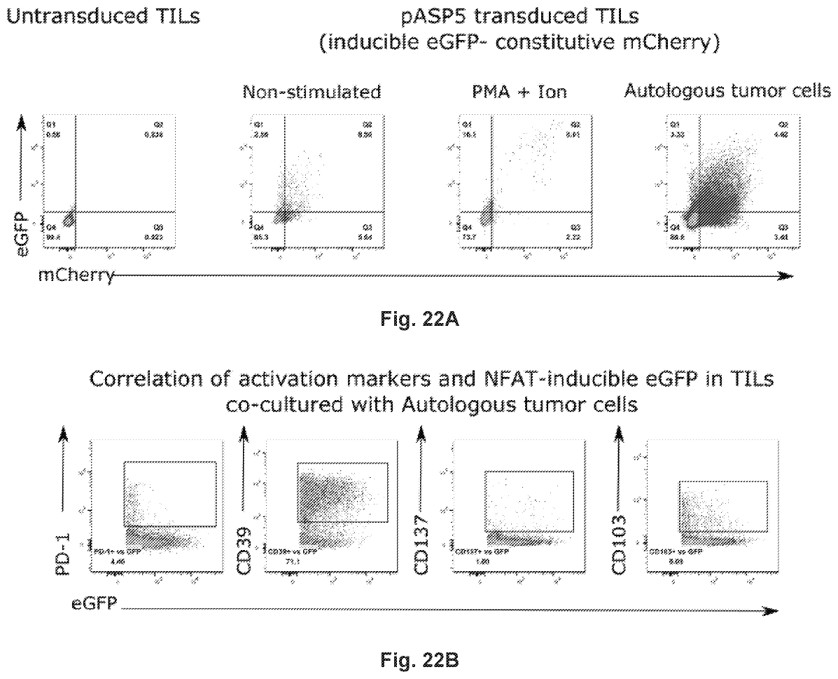

The single-vector platform can be used as a high-throughput and quantitative screening approach for identification of antigen specific immune cells, such as CAR T cells, TCR modified cells or endogenous TILs. Here successful transduction of patient Tumor-Infiltrating Lymphocytes (TILs) with pASP5 (inducible eGFP+constitutive mCherry) was demonstrated. The inducible module was upregulated in chemically stimulated TILs demonstrating a proof-of-principle to monitor NFAT-mediated signaling in patient derived TILs. When TILs were co-cultured with matching autologous tumor sample, inducible eGFP signature was increased (FIG. 22A). Correlation between standard activation markers used in determining antigen-specific immune cells and NFAT-inducible eGFP was also investigated. Correlation was observed with CD39, CD137 and CD103 but not PD-1 (FIG. 22B).

### Example 20—IL13Ra2-Targeting CAR-Inducible Secretion of Functional Bispecific T Cell Engager pASP79 (BiTE)

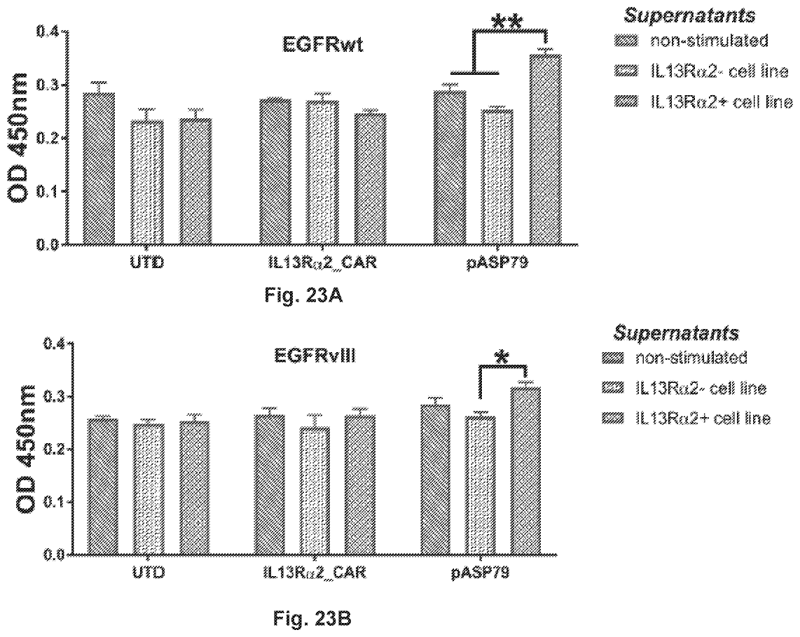

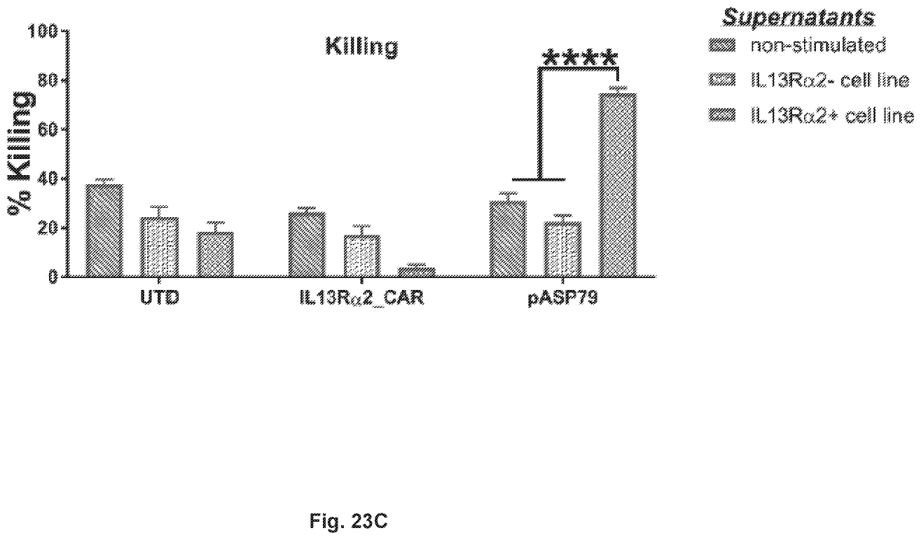

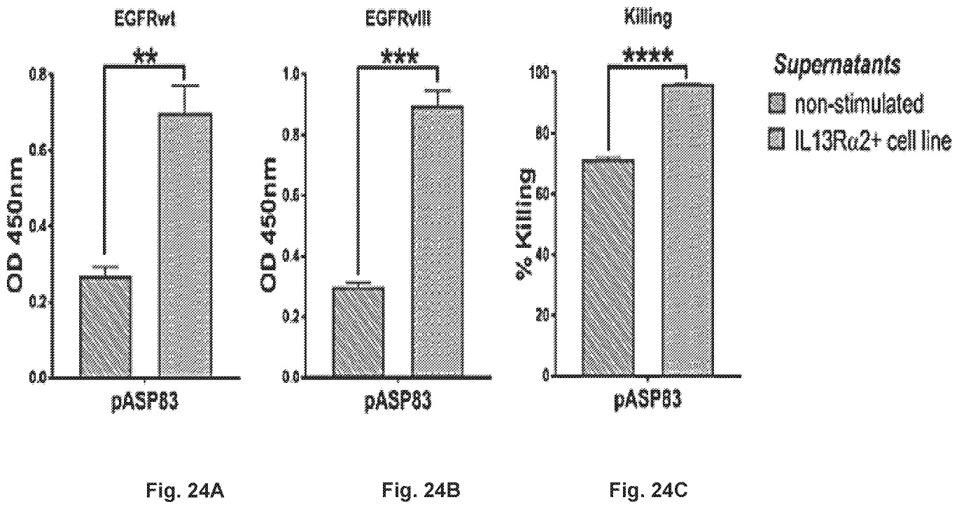

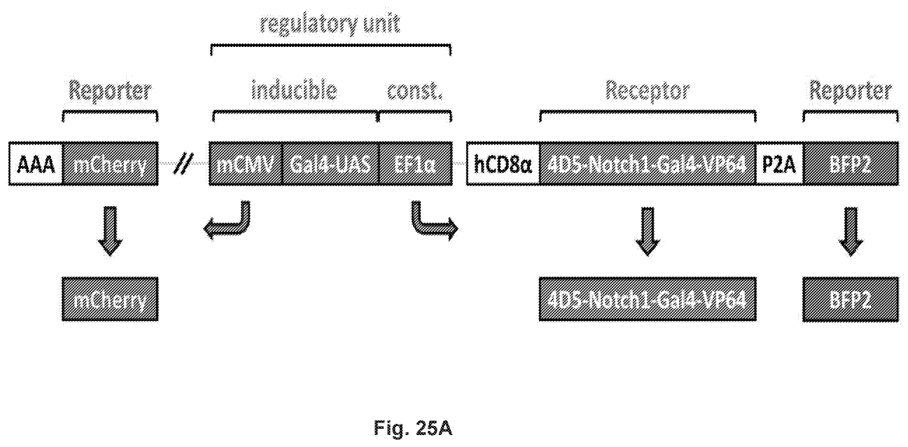

To demonstrate implementation of the single-vector system using bispecific T cell engagers (BiTEs), we tested constructs combining constitutive expression of IL13Rα2-targeting CAR with 2 different inducible BiTEs (pASP79 and pASP83; both BiTEs can target both EGFR wild type (EGFRwt) and EGFR variant III (EGFRvIII) proteins). When engineered cells were co-cultured with an IL13Rα2 expressing cell line, inducible expression of soluble BiTEs was observed (FIG. 23A, FIG. 23B and FIG. 24A-24B). Functionality of the CAR T cell-secreted BiTEs was demonstrated by specific lysis of EGFRwt and EGFRvIII expressing target cells when supernatants from antigen activated CAR T cells were added to co-culture of un-transduced primary T cells and target cells (FIG. 23C and FIG. 24C). Thus, pASP79 and pASP83 CAR T cells secrete biologically active EGFR-specific BiTEs in response to antigen activation, and the secreted BiTEs can redirect the activity of un-transduced T cells against EGFR-expressing cancer cells.

### Example 21—Antigen-Inducible Transcriptional Factors TCF-7 and FOXO1-3A Mediate Less Differentiated Status and Improved Antigen-Dependent Proliferation in CAR T Cells

CAR T cells are prepared as follows: Primary T cells are activated, transduced with the control construct pASP30 (inducible eGFP+constitutive 4D5 anti-Her2 scFv 4-1BB+CD3-zeta CAR; back-to-back orientation), and constructs pASP72 (inducible hTCF-7+constitutive 4D5 anti-Her2 scFv 4-1BB+CD3-zeta CAR; back-to-back orientation) and pASP73 (inducible human FOXO1-3A+constitutive 4D5 anti-Her2 scFv 4-1BB+CD3-zeta CAR; back-to-back orientation) at MOI5, expanded and rested according to the standard protocol. Engineered primary T cells will be injected intravenously (i.v.) at 3 different doses (0.15/0.3/1 million of CAR+ cells per mouse) into NSG mice bearing subcutaneous (s.c.) xenograft SKOV3 tumors. 1 million SKOV3 cells (engineered to express luciferase) will be injected s.c. 4 days prior to the administration of CART cells to establish tumors.

Tumor growth is monitored by imaging, quantification of luciferase activity, caliper measurements and survival. Blood counts of human primary T cells in mice will be monitored. Using multicolor flow cytometry, phenotype of engineered cells is investigated.

In Her2-targeting CAR T cells engineered with inducible transcriptional factors, improved tumor control and less differentiated and less exhausted phenotype of CAR T cells is expected to be observed. In the FOXO1-3A example, increased memory component is also expected to be observed. Improved persistence of CAR T cells is expected. Tumor re-challenge model is performed, where better response when transcriptional factor will be engineered into primary T cells along with a CAR, using single vector system is observed.

Along with inducible transcriptional factors, constitutive expression of the same genes as a control is observed. More profound effects on the phenotype are expected to be observed in this case but also increased risk of malignant transformations of engineered cells with certain transcriptional factors.

### Example 22—Use of the Single-Vector System to Screen for Immunomodulatory Molecules

The single-vector system is utilized to combine solid tumor-targeting CARs with inducible expression of a candidate immunomodulatory molecule. Individual constructs are prepared comprising a CAR, for example a Her2-specific CAR, in combination with a candidate immunomodulatory molecule, and primary T cells are transduced. The functionalities of CAR T cells so generated is determined ex vivo in normal and immunosuppressive conditions. Inducible, antigen-driven expression of integrated molecules is validated on the transcriptional (RT-qPCR) and protein level (ELISA and Flow Cytometry). Their biological activity is further validated. For example, the capacity to lyse Her2 positive target cell line SKOV3 is measured for Her2-specific CAR T cells. Cytokine profiles released by activated CAR T cells are examined using for example the Luminex system. Her2 negative cell line MDA468 may serve as a negative control. The phenotype of CAR T cells is measured by multi-color flow cytometry.

### Example 23—synNotch Single-Vector System

The synNotch receptor is comprised of a portion of Notch wherein both the extracellular and intracellular domains have been completely replaced, leaving only the small central regulatory region (Morsut et al., Cell 164, 780-791, Feb. 11, 2016). The extracellular and intracellular domains from Notch can be swapped with diverse domains. On the extracellular side, different antigen binding domains can be linked to Notch (e.g. scFvs). On the intracellular side, different effectors can be used (e.g. transcriptional activators or transcriptional repressors). Examples of transcriptional activators and repressors that can be used with the synNotch system include but are not limited to Gal4-VP64, tetR-VP64 (tTA), ZFHD1-VP64, and Gal4-KRAB. Upon binding of the extracellular antigen binding domain to its target molecule, Notch is cleaved which releases the intracellular domain containing the transcriptional activator/repressor which leads to the activation/repression of downstream genes.

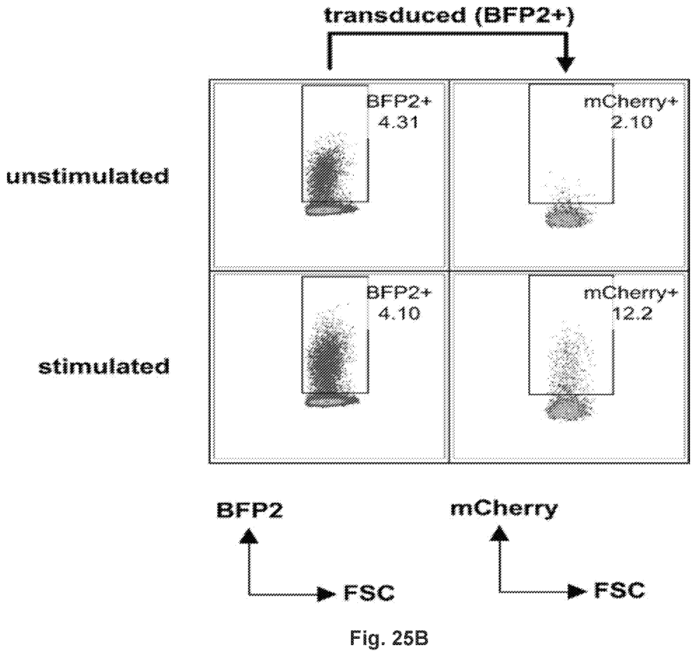

Primary human T cells were transduced with the synNotch single-vector construct shown in FIG. 25A. The constitutive EF1alpha promoter leads to the continuous expression of a HER2-specific synNotch receptor (4D5-Notch1-Gal4-VP64) and a blue fluorescent BFP2 reporter protein. Binding of the synNotch receptor to its target antigen HER2 induces Notch1 cleavage which releases the transcription factor Gal4-VP64 from the receptor. Gal4-VP64 binds to the Gal4 enhancer sequence (Gal4-UAS) thereby activating the minimal CMV promoter (mCMV) and leading to the production of a red fluorescent mCherry reporter protein.

Transduced T cells were cultured in the absence or in the presence of HER2-expressing SKOV3 tumor cells for 24 hours and analyzed with a LSRFortessa flow-cytometer (BD Biosciences). FIG. 25B shows experimental validation of the synNotch single-vector system. Representative flow-cytometry plots were generated showing expression of BFP2 and mCherry in primary human T cells transduced with the synNotch single-vector construct.

Sequences
Regulatory unit (DNA sequence)
(SEQ ID NO: 34)
AGGCTGGATCGGTCCCGGTGTCTTCTATGGAGGTCAAAACAGCGTGGA
TGGCGTCTCCAGGCGATCTGACGGTTCACTAAACGAGCTCTGCTTATA
TAGGCCTCCCACCGTACACGCCTACGGAGGACAGTACTCCGTGCGGAG
GACAGTACTCCGTCCGGAGGACAGTACTCCGATCGGAGGACAGTACTC

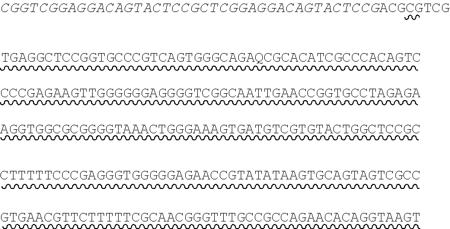

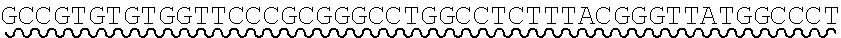

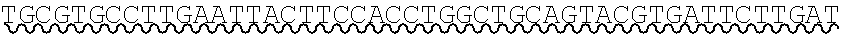

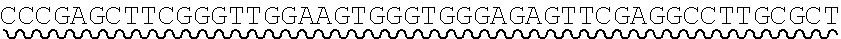

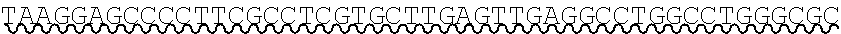

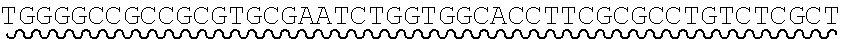

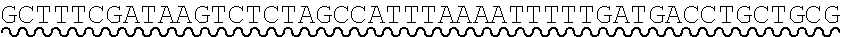

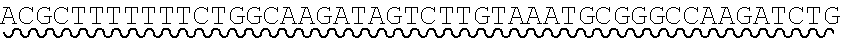

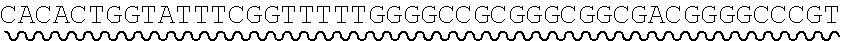

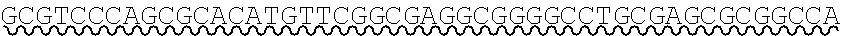

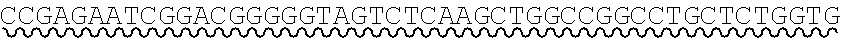

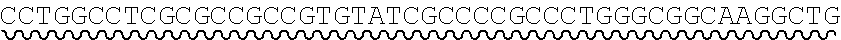

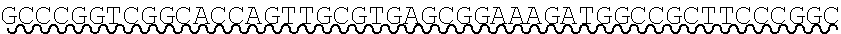

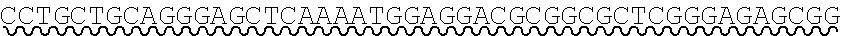

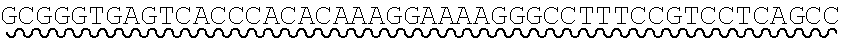

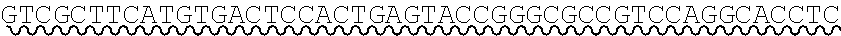

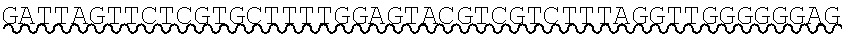

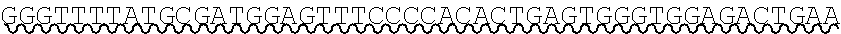

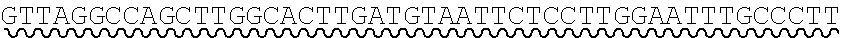

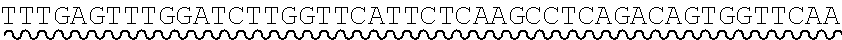

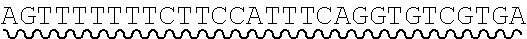

mCMV (rev): bold
Ga14-UAS: italics
EFlalpha (fwd): wave underline
mCherry reporter (protein sequence)
(SEQ ID NO: 35)
MVSKGEEDNMAIIKEFMRFKVHMEGSVNGHEFEIEGEGEGRPYEGTQT
AKLKVTKGGPLPFAWDILSPQFMYGSKAYVKHPADIPDYLKLSFPEGF
KWERVMNFEDGGVVTVTQDSSLQDGEFIYKVKLRGTNFPSDGPVMQKK
TMGWEASSERMYPEDGALKGEIKQRLKLKDGGHYDAEVKTTYKAKKPV
QLPGAYNVNIKLDITSHNEDYTIVEQYERAEGRHSTGGMDELYK
4D5-Notch1-Ga14-VP64 receptor (protein sequence)
(SEQ ID NO: 36)
DIQMTQSPSSLSASVGDRVTITCRASQDVNTAVAWYQQKPGKAPKLLI
YSASFLYSGVPSRFSGSRSGTDFTLTISSLQPEDFATYYCQQHYTTPP
TFGQGTKVEIKRTGSTSGSGKPGSGEGSEVQLVESGGGLVQPGGSLRL
SCAASGFNIKDTYIHWVRQAPGKGLEWVARTYPTNGYTRYADSVKGRF
TISADTSKNTAYLQMNSLRAEDTAVYYCSRWGGDGFYAMDVWGQGTLV
TVSSILDYSFTGGAGRDIPPPQIEEACELPECQVDAGNKVCNLQCNNH
ACGWDGGDCSLNFNDPWKNCTQSLQCWKYFSDGHCDSQCNSAGCLFDG
FDCQLTEGQCNPLYDQYCKDHFSDGHCDQGCNSAECEWDGLDCAEHVP
ERLAAGTLVLVVLLPPDQLRNNSFHFLRELSHVLHTNVVFKRDAQGQQ
MIFPYYGHEEELRKHPIKRSTVGWATSSLLPGTSGGRQRRELDPMDIR
GSIVYLEIDNRQCVQSSSQCFQSATDVAAFLGALASLGSLNIPYKIEA
VKSEPVEPPLPSQLHLMYVAAAAFVLLFFVGCGVLLSRKRRRMKLLSS
IEQACDICRLKKLKCSKEKPKCAKCLKNNWECRYSPKTKRSPLTRAHL
TEVESRLERLEQLFLLIFPREDLDMILKMDSLQDIKALLTGLFVQDNV
NKDAVTDRLASVETDMPLTLRQHRISATSSSEESSNKGQRQLTVSAAA
GGSGGSGGSDALDDFDLDMLGSDALDDFDLDMLGSDALDDFDLDMLGS
DALDDFDLDMLGS
BFP2 reporter (protein sequence)
(SEQ ID NO: 37)
MSELIKENMHMKLYMEGTVDNHHFKCTSEGEGKPYEGTQTMRIKVVEG
GPLPFAFDILATSFLYGSKTFINHTQGIPDFFKQSFPEGFTWERVTTY
EDGGVLTATQDTSLQDGCLIYNVKIRGVNFTSNGPVMQKKTLGWEAFT
ETLYPADGGLEGRNDMALKLVGGSHLIANAKTTYRSKKPAKNLKMPGV
YYVDYRLERIKEANNETYVEQUEVAVARYCDLPSKLGHKLN

### Example 24

Constructs are made that express a CAR and a transcription factor under control of a single constitutive promoter. Some constructs include a 2A peptide spacer between the CAR and the transcription factor. Immune cells, for example T cells, are transduced with the constructs.

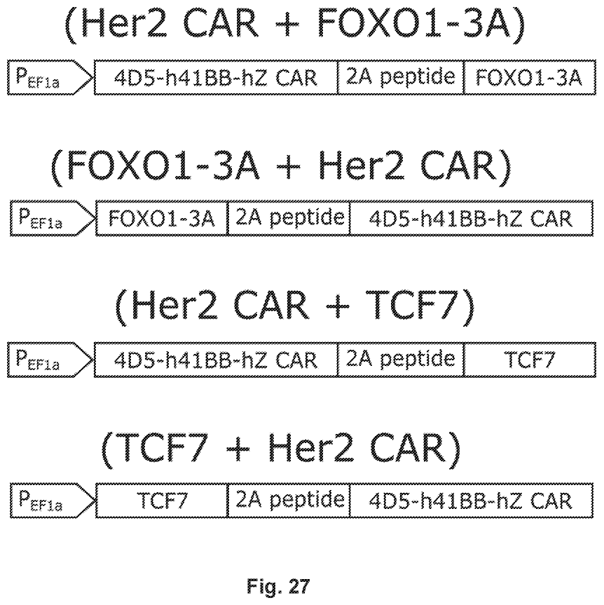

FIG. 27 illustrates constructs comprising a CAR and a transcription factor under control of a single constitutive promoter with a 2A peptide spacer between the CAR and the transcription factor.

Constructs are made that express a CAR under control of a first constitutive promoter and a transcription factor under control of a second constitutive promoter. Immune cells, for example T cells, are transduced with the constructs.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A single viral vector comprising:
a first polynucleotide comprising a constitutive promoter operably linked to a nucleic acid encoding at least one transgene, wherein one of the at least one transgenes encodes a receptor or receptor subunit, a receptor fusion protein or a fluorescent marker; and
a second polynucleotide comprising an inducible promoter operably linked to a nucleic acid encoding an effector.

2. The viral vector of claim 1,
(i) wherein the transgene encodes a receptor fusion protein comprising an antigen-specific synNotch receptor and a reporter;
(ii) wherein the transgene encodes a receptor or receptor subunit that is a TCR or TCR subunit, wherein the TCR comprises an extracellular antigen binding domain capable of specifically binding a cognate antigen and an intracellular domain, wherein the intracellular domain drives expression of the effector when the antigen-binding domain of the TCR binds its cognate antigen by activating the inducible promoter operably linked to the effector;
(iii) wherein the transgene encodes a receptor or receptor subunit that is receptor or receptor subunit is a cytokine receptor or cytokine receptor subunit, wherein the cytokine receptor subunit is IL-6Ra;
(iv) wherein the transgene encodes a receptor fusion protein that is a chimeric antigen receptor (CAR) comprising an extracellular antigen-binding domain capable of specifically binding a cognate antigen and an intracellular signaling domain, wherein the inducible promoter is capable of driving expression of the effector when the antigen binding domain binds its target, wherein the intracellular signaling domain drives expression of the effector when the antigen-binding domain of the CAR binds its cognate antigen by activating the inducible promoter operably linked to the effector, wherein the inducible promoter is capable of driving expression of the effector when the receptor binds its ligand;
(v) wherein the transgene encodes a receptor fusion protein that is a chimeric antigen receptor (CAR) comprising an extracellular antigen-binding domain capable of specifically binding a cognate antigen and an intracellular signaling domain; or
(vi) wherein the transgene encodes a fluorescent marker, wherein the fluorescent marker is mCherry.

3-15. (canceled)

16. The viral vector of claim 1,
(i) wherein the first polynucleotide, the second polynucleotide or both the first and the second polynucleotides comprise an insulator nucleic acid sequence or a linker nucleic acid sequence;
(ii) wherein the constitutive promoter comprises a promoter selected from the group consisting of an EF-1alpha promoter, a PGK-1 promoter, an UBC promoter, a CMV promoter, a CAGG promoter and an SV40 promoter;
(iii) wherein the inducible promoter comprises a promoter selected from the group consisting of an NFAT promoter, a STAT3-sensing promoter, a CD69 promoter, a CD137 promoter or a hypoxia responsive element promoter and a minimal promoter operably linked to an inducible enhancer;
(iv) wherein the inducible promoter is linked to a minimal promoter (P_{MIN});
(v) wherein the effector is a cytokine, an interferon, a chemokine, an antibody or antibody fragment, a bispecific antibody, a BiTE, a checkpoint inhibitor antagonist, an enzyme, a regulatory element, dCas9, a transcription factor or a DNA binding domain of a transcription factor, wherein the receptor fusion protein comprises a transcription factor and a transcription activator, wherein the transcription activator is VP64; or
(vi) wherein the effector is a fusion protein comprising at least two components, wherein each component is selected from the group consisting of a cytokine, an interferon, a chemokine, an antibody or antibody fragment, a bispecific antibody, a BiTE, a checkpoint inhibitor antagonist, an enzyme, a regulatory element, a transcription factor or a DNA binding domain of a transcription factor, wherein the at least two components are linked through a 2A peptide.

17-23. (canceled)

24. The viral vector of claim 16,
(i) wherein the cytokine is IL-2, IL-12, IL-15, IL-18 or IL-21;
(ii) wherein the interferon is interferon alpha or interferon beta;
(iii) wherein the antibody or antibody fragment is anti-IL-6, anti-IL-6R, anti-IL-6Ra, anti-TNFalpha, anti-IL-1, anti-PD1, anti-CD40, anti-CD25, anti-CD3, anti-CD20, anti-IL-8, anti-MCP1, anti-MIP-1, anti-TGFβ, anti-CD47, anti-CSF1R, anti-CD28, anti-TIGIT, anti-VEGFR or anti-FAP;
(iv) wherein the antibody or antibody fragment is agonistic;
(v) wherein the checkpoint inhibitor antagonist is anti-PD-L1, anti-PD-1, anti-CTLA4, anti-LAG3, anti-TIM3, anti-2B4 or anti-CD160 or anti-CD5;
(vi) wherein the bispecific antibody comprises an antigen binding domain of at least one of anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-CD40 antagonistic antibody, anti-IL-8, anti-MCP1, anti-MIP-1, anti-TGFβ, anti-CD47, anti-CSF1R, anti-CD28, anti-TIGIT, anti-VEGFR, anti-FAP, anti-PD-L1, anti-PD-1, anti-CTLA4, anti-LAG3, anti-TIM3, anti-2B4, anti-CD160 or anti-CD5, or combinations thereof;
(vii) wherein the enzyme is heparinase, collagenase or a metalloproteinase;
(viii) wherein the chemokine is CCL5 (RANTES), XCL-1, XCL-2, CCR-7, CCL-19 or CCL-21; or
(ix) wherein the regulatory element, transcription factor or portion thereof is T-bet, TCF7, EOMES, a Runx family member, BLIMP1, Bc12, Bc16, FoxP3, FoxO1, FoxO1-3A, or a portion thereof, wherein the Runx family member is Runx1 or Runx3.

25-33. (canceled)

34. The viral vector of claim 1,
(i) wherein the viral vector is a retroviral vector, an adenoviral vector or an adeno-associated viral vector;
(ii) wherein the viral vector is a lentiviral vector;
(iii) wherein the inducible promoter and the constitutive promoter drive transcription in the same direction, or in different directions; or
(iv) wherein the CAR comprises an antigen binding domain, a transmembrane domain, and an intracellular signaling domain, wherein the intracellular signaling domain comprises one or more costimulatory signaling domains and an immunoreceptor tyrosine activation motif-containing signaling domain, wherein the one or more costimulatory signaling domains comprise the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and wherein the immunoreceptor tyrosine activation motif-containing signaling domain comprises a CD3 zeta signaling domain.

35-40. (canceled)

41. An engineered cell comprising the viral vector of claim 1.

42-44. (canceled)

45. A method for generating an engineered cell, comprising introducing the viral vector of claim 1 into a cell.

46-50. (canceled)

51. A method for treating a patient having a disease, disorder or condition associated with expression of an antigen, the method comprising administering to the patient an effective amount of a composition comprising the engineered cell of claim 41.

52-55. (canceled)

56. An engineered immune cell or engineered immune cell precursor cell, comprising:
an insertion in a gene locus,
wherein the insertion comprises a first polynucleotide comprising a constitutive promoter operably linked to a nucleic acid encoding at least one transgene, wherein one of the at least one transgenes encodes a receptor or receptor subunit, a receptor fusion protein or a fluorescent marker; and
a second polynucleotide comprising an inducible promoter operably linked to a nucleic acid encoding an effector.

57. The engineered cell of claim 56,
(i) wherein the transgene encodes a receptor fusion protein comprising an antigen-specific synNotch receptor fusion protein and a reporter;
(ii) wherein the transgene encodes a receptor or receptor subunit that is a TCR or TCR subunit, wherein the TCR comprises an extracellular antigen binding domain capable of specifically binding a cognate antigen and an intracellular domain, wherein the intracellular domain drives expression of the effector when the antigen-binding domain of the TCR binds its cognate antigen by activating the inducible promoter operably linked to the effector;
(iii) wherein the transgene encodes a receptor or receptor subunit that is a cytokine receptor or cytokine receptor subunit, wherein the cytokine receptor subunit is IL-6R;
(iv) wherein the transgene encodes a receptor fusion protein that is a CAR comprising an extracellular antigen-binding domain capable of specifically binding a cognate antigen and an intracellular signaling domain, wherein the inducible promoter is capable of driving expression of the effector when the antigen binding domain binds its target, wherein the intracellular signaling domain drives expression of the effector when the antigen-binding domain of the CAR binds its cognate antigen by activating the inducible promoter operably linked to the effector, wherein the inducible promoter is capable of driving expression of the effector when the receptor binds its ligand;
(v) wherein the transgene encodes a receptor fusion protein that is a CAR comprising an extracellular antigen-binding domain capable of specifically binding a cognate antigen and an intracellular signaling domain; or
(vi) wherein the transgene encodes a fluorescent marker, wherein the fluorescent marker is mCherry.

58-70. (canceled)

71. The engineered cell of claim 56,
(i) wherein the first polynucleotide, the second polynucleotide or both the first and the second polynucleotides comprise an insulator nucleic acid sequence or a linker nucleic acid sequence;
(ii) wherein the insertion is encoded in a donor template, wherein the donor template is encoded in a viral vector;
(iii) wherein the gene locus encodes a TCR subunit, an HLA or an immune checkpoint molecule, wherein the immune checkpoint molecule is PD-1, TIM3, LAG3, CTLA4, 2B4, CD160 or CD5;
(iv) wherein the insertion in a gene locus is mediated by a CRISPR-related system, wherein the CRISPR-related system is CRISPR/Cas9;
(v) wherein the constitutive promoter comprises a promoter selected from the group consisting of an EF-1alpha promoter, a PGK-1 promoter, an UBC promoter, a CMV promoter, a CAGG promoter or an SV40 promoter;
(vi) wherein the inducible promoter comprises a promoter selected from the group consisting of an NFAT promoter, a STAT3-sensing promoter, a CD69 promoter, a CD137 promoter, a hypoxia-responsive element promoter or a minimal promoter linked to an inducible enhancer;
(vii) wherein the inducible promoter is linked to a minimal promoter (P_{MIN});
(viii) wherein the effector is a cytokine, an interferon, a chemokine, an antibody or antibody fragment, a bispecific antibody, a BiTE, a checkpoint inhibitor antagonist, an enzyme, a regulatory element, dCas9, a transcription factor or a DNA binding domain of a transcription factor; or
(ix) wherein the effector is a fusion protein comprising at least two components, wherein each component is selected from the group consisting of a cytokine, an interleukin, an interferon, a chemokine, an antibody or antibody fragment, a bispecific antibody, a checkpoint inhibitor antagonist, an enzyme, a regulatory element, a transcription factor, a DNA binding domain of a transcription factor, wherein the at least two components are linked through a 2A peptide.

72-81. (canceled)

82. The engineered cell of claim 71,
(i) wherein the fusion protein comprises a transcription factor and a transcription activator, wherein the transcription activator is VP64;
(ii) wherein the cytokine is IL-2, IL-12, IL-15, IL-18 or IL-21;
(iii) wherein the interferon is interferon alpha or interferon beta;
(iv) wherein the antibody or antibody fragment is anti-IL-6, anti-IL-6R, anti-IL-6Ra, anti-TNFalpha, anti-IL-1, anti-PD1, anti-CD40, anti-CD25, anti-CD3, anti-CD20, anti-IL-8, anti-MCP1, anti-MIP-1, anti-TGFβ, anti-CD47, anti-CSF1R, anti-CD28, anti-TIGIT, anti-VEGFR or anti-FAP;
(v) wherein the antibody or antibody fragment is agonistic;
(vi) wherein the checkpoint inhibitor antagonist is anti-PD-L1, anti-PD-1, anti-CTLA4, anti-LAG3, anti-TIM3, anti-2B4 or anti-CD160 or anti-CD5;
(vii) wherein the bispecific antibody comprises an antigen binding domain of at least one of anti-IL-6, anti-IL-6R, anti-IL-6Ra, anti-TNFalpha, anti-IL-1, anti-PD1, anti-CD25, anti-CD3, anti-CD20, anti-CD40 agonistic antibody, anti-CD40 antagonistic antibody, anti-IL-8, anti-MCP1, anti-MIP-1, anti-TGFβ, anti-CD47, anti-CSF1R, anti-CD28, anti-TIGIT, anti-VEGFR, anti-FAP, anti-PD-L1, anti-PD-1, anti-CTLA4, anti-LAG3, anti-TIM3, anti-2B4, anti-CD160 or anti-CD5, or combinations thereof;
(viii) wherein the enzyme is heparinase, collagenase or a metalloproteinase;
(ix) wherein the chemokine is CCL5 (RANTES), XCL-1, XCL-2, CCR-7, CCL-19 or CCL-21; or
(x) wherein the regulatory element, transcription factor or portion thereof is T-bet, TCF7, EOMES, a Runx family member, BLIMP1, Bcl2, Bcl6, FoxP3, FoxO1 or FoxO1-3A, or a portion thereof, wherein the Runx family member is Runx1 or Runx3.

83-94. (canceled)

95. The engineered cell of claim 56,
(i) wherein the viral vector is a retroviral vector, an adenoviral vector, an adeno-associated viral vector;
(ii) wherein the viral vector is a lentiviral vector;
(iii) wherein the inducible promoter and the constitutive promoter drive transcription in the same direction, or in different directions; or
(iv) wherein the CAR comprises an antigen binding domain, a transmembrane domain, and an intracellular signaling domain, wherein the intracellular signaling domain comprises one or more costimulatory signaling domains and an immunoreceptor tyrosine activation motif-containing signaling domain, wherein the one or more costimulatory signaling domains comprises the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, wherein the immunoreceptor tyrosine activation motif-containing signaling domain comprises a CD3 zeta signaling domain.

96-101. (canceled)

102. The engineered cell of claim 56, wherein the engineered cell is an NK cell, a B cell, a dendritic cell or a macrophage, or a precursor of an NK cell, a precursor of a B cell, a precursor of a dendritic cell or a precursor of a macrophage.

103. A method for treating a patient having a disease, disorder or condition associated with expression of an antigen, the method comprising administering to the patient an effective amount of a composition comprising the engineered cell of claim 56.

104. (canceled)

105. A viral vector comprising:
a first polynucleotide comprising a first constitutive promoter and a nucleic acid encoding at least one transgene, wherein the first constitutive promoter is operably linked to the nucleic acid encoding the at least one transgene, wherein one of the at least one transgenes is a receptor, a receptor fusion protein or a fluorescent marker; and
a second polynucleotide comprising a second constitutive promoter and a nucleic acid encoding an effector, wherein the second constitutive promoter is operably linked to the nucleic acid encoding the effector.

106. The viral vector of claim 105,
(i) wherein the transgene encodes a receptor fusion protein;
(ii) wherein the transgene encodes a receptor or receptor subunit that is a TCR or TCR subunit, wherein the receptor is a TCR, wherein the TCR comprises an extracellular antigen binding domain capable of specifically binding a cognate antigen and an intracellular domain;
(iii) wherein the transgene encodes a receptor or receptor subunit that is a cytokine receptor or a cytokine receptor subunit, wherein the cytokine receptor subunit is IL-6Ra;
(iv) wherein the transgene encodes a receptor fusion protein that is a CAR comprising an extracellular antigen-binding domain capable of specifically binding a cognate antigen and an intracellular signaling domain; or
(v) wherein the transgene encodes a fluorescent marker, wherein the fluorescent marker is mCherry.

107-114. (canceled)

115. The viral vector of claim 105,
(i) wherein the first polynucleotide, the second polynucleotide or both the first and the second polynucleotides comprise an insulator sequence or a linker sequence;
(ii) wherein the first and/or second constitutive promoter is an EF-1alpha promoter, a PGK-1 promoter, an UBC promoter, a CMV promoter, a CAGG promoter or an SV40 promoter; or
(iii) wherein the effector is a cytokine, an interferon, a chemokine, an antibody or antibody fragment, a bispecific antibody, a checkpoint inhibitor antagonist, an enzyme, a regulatory element, dCas9, a transcription factor or a DNA binding domain of a transcription factor.

116-117. (canceled)

118. An engineered immune cell comprising the vector of claim 105.

119. An engineered immune cell or engineered immune cell precursor cell, comprising:
an insertion in a gene locus,
wherein the insertion comprises a first polynucleotide comprising a first constitutive promoter and a nucleic acid encoding at least one transgene, wherein the first constitutive promoter is operably linked to the nucleic acid encoding the at least one transgene,
wherein one of the at least one transgenes encodes a receptor, a receptor fusion protein or a fluorescent marker; and
a second polynucleotide comprising a second constitutive promoter and a nucleic acid encoding an effector, wherein the second constitutive promoter is operably linked to the nucleic acid encoding the effector.

120. The engineered cell of claim 119, wherein the transgene encodes a receptor fusion protein.

121. The engineered cell of claim 119, wherein the transgene encodes a receptor or receptor subunit.

122. The engineered cell of claim 119, wherein the effector is a cytokine, an interferon, a chemokine, an antibody or antibody fragment, a bispecific antibody, a checkpoint inhibitor antagonist, an enzyme, a regulatory element, dCas9, a transcription factor or a DNA binding domain of a transcription factor.

123. A viral vector comprising:
a polynucleotide comprising a constitutive promoter, a nucleic acid encoding a first transgene, and a nucleic acid encoding a second transgene, wherein the constitutive promoter is operably linked to the nucleic acid encoding the first transgene and the second transgene;
wherein the first transgene is a receptor or receptor subunit, a receptor fusion protein or a fluorescent marker; and
wherein the second transgene is an effector.

124. The viral vector of claim 123, wherein the receptor fusion protein is a CAR.

125. The viral vector of claim 123, wherein the effector is a transcription factor.

126. An engineered immune cell or engineered immune cell precursor cell, comprising:
an insertion in a gene locus,
wherein the insertion comprises a polynucleotide comprising a constitutive promoter, a nucleic acid encoding a first transgene, and a nucleic acid encoding a second transgene, wherein the constitutive promoter is operably linked to the nucleic acid encoding the first transgene and the second transgene;
wherein the first transgene is a receptor or receptor subunit, a receptor fusion protein or a fluorescent marker; and
wherein the second transgene is an effector.

127. The engineered cell of claim 126, wherein the receptor fusion protein is a CAR.

128. The engineered cell of claim 126, wherein the effector is a transcription factor.


SEQUENCE LISTING (37 sequences)

SEQ ID NO: 1 - DNA, 6 bp, Artificial Sequence
  Synthetic Construct
catatg

SEQ ID NO: 2 - DNA, 6 bp, Artificial Sequence
  Synthetic Construct
cccggg

SEQ ID NO: 3 - DNA, 264 bp, Artificial Sequence
  Synthetic Construct
ggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccgg

SEQ ID NO: 4 - DNA, 4908 bp, Artificial Sequence
  pASP73 (iFOX01-3A + Her2 CAR)
ttagccggaaacccagctgtgtgtggtggttttcacgctgtgagggaagctctggttgggcagcacgttatcgaagttgaagtccagggtatcgccgtccatcaggtcgttgcggatgatggattccatgtcgcagtccagtctctcgatgaacatgccgtccagatcagagggcagcttctcttggtgcagcaggcccattctgccgtagccattacagctggacacgctggagtagccgccgagggcagacatctgcataggatgaggcagtgggacctgcacaggggttttcacttgggtcagccggttcatgccgctggtatgaggcatggtggacacggtgtgaggcagaggtctgccattaacggcgcttgtctgctgggcatgtccagggtgtgtgtggctgctggggttcatcatcttgttgtggctggcctgagagccgtatgtgctcatcacgctattggggcccatcatcacgttctggcccagcactctactattaggctgggccacgccagggtccacaggtgtcatgatgtcgttgtgaggagggctgtcagaggtcagcagctctttcagcagtccaggggcacaattgtactggctcatgccgccgtaggaggacttgttgtcctgcagggtctgaatgggcatctgtggcagagggctcatgctgctctggccgtaggtgtacttctggtagttaggagatgggctgttcaggcttgtgttaggaggggcgaagctgtagcaaggggtctgctgcatcatggtgccggggctgctttgtgtagacacggtcaggctggtagggctgctcagcagattcaggttgtccagcaggttttccatgttctcggggttgctgatctcgctcaggcttggcagtgtgctggccattttggcggcgcttggaggatacaccatgctgtgcacgtcgccttctccgagatcatcctgctcggtcatgatggggctcagtctgccagagatggtgctggcattggcgctggttctgggtctgaaggtggaccagttgtcgaagtcgtcgttgctgtgagagccaggagaggcgggccacttagaaaactggctgccaggagaatcgccagcgccttcttgtccagactgcagagaggccttcttcttggcggctctgcttctgctcttggcgaacttggagttgttgtccatggcagcggcccttctacgaggagacttgccgctcttgccgccttcgggattcagcatccaccaagaggacttgccggtgccttcattctgcacccggatgaacttgctgtgcagggacagattgtgccggatgctgttcttccagccggcggagctattgctgtcgcccttgtccttgaagtagggcacgctcttgaccatccactcgtagatctggctcagggtcagtctcttctcggcgctgctctcaatggccttggtgatcaggtcggcgtagctcagattgccccaggcgtttcttctgctggagctgctctttctaggctgtccagcaagtggtccagcggcagctggaggaacaggaggatgctgagacagaggtccaggtggaggtggttgaggtggagcagggtgcagacatccggcctcaggtccctgaaaatcgccgcaaagtccacctgtagcagcagctgcggctgcggcggcaacagctgcagccacagatccaggagcctgagggaagtcctcgctctcttccagcaggctcaggttgctcatgaagtcggcggacactgcagcagcagaggcagaaggcagtccagcagcagcatcaggattagcggcggcagatccgctaggagcagggctagatgtggcgctgttgctctggctgaactcaggtctaggcagaggccaagcacaagatctaggccgaggcaggggctcgaaatcagggtcgatttccaccacctggggagcctcggccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 5 - DNA, 180 bp, Artificial Sequence
  Synthetic NFAT promoter
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgt

SEQ ID NO: 6 - DNA, 32 bp, Artificial Sequence
  Minimal promoter
tagagggtatataatggaagctcgacttccag

SEQ ID NO: 7 - DNA, 1184 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 29 bp, Artificial Sequence
  Spacer + Kozak sequence
ggcattccggtactgttggtaaagccacc

SEQ ID NO: 9 - DNA, 253 bp, Artificial Sequence
  Synthetic Construct
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttagagggtatataatggaagctcgacttccagcccgggggcattccggtactgttggtaaagccacc

SEQ ID NO: 10 - DNA, 2936 bp, Artificial Sequence
  pASP4.2
tcgagttaattaagctagcccggtactgttggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttagagggtatataatggaagctcgacttccagcccgggggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 11 - DNA, 2936 bp, Artificial Sequence
  pASP5
tcgagttaattaagctagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 12 - DNA, 3158 bp, Artificial Sequence
  pASP7
tcgagtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaattaagctagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 13 - DNA, 3413 bp, Artificial Sequence
  pASP8
tcgagttaattaagctagcaaggctttctgtttcctgcattcttacttcttactacgtgatacatctagtcaccagggaagaagcgaatgacacacttccaaaaaccaattcgtagctttctaaataaaaccctttctagctggagagagatccatgagcatagagatcttaaaattcatgttcagcaataaatcctggggccccagacagtgtcaggtgcatagggggtgttcagtaaatatcagttaaatgttgcataaatccgataaacgggattcctggaaaatactacactctccttctccaaattatcttcatctcaaagacaggaacctctaacttttaattctttacttagattatgctgtctcctaaactgtttatgttttctgaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcacagactcaacaagagctccagcaaagactttcactgtagcttgacttgacctgagattaactagggaatcttgagaataaagcccgggggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 14 - DNA, 3031 bp, Artificial Sequence
  pASP9
tcgagttaattaagctagcaatgctttgtcctggagagctatcttaagggacaaaatcgttttcccagcgtcatctgtgacacatcctgacagtagagagctgcttccaagaagcaatttgaagtgccattatcaggcagggacgggggctctaggggatttcggggtcagcagatatgaaatgaatgatttcatagggctgtcacagagctgtggtgggaatttcccatgagaccccgcccctggctgagtcaccgcactcctgtgtttgacctgaagtcctctcgtgctgcagaagcctgaagaccaaggagtggaaagttctccggcagccctgagatctcccgggggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 15 - DNA, 3815 bp, Artificial Sequence
  pASP18
tcgagttaattaagctagcccggtactgttggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttagagggtatataatggaagctcgacttccagcccgggggcattccggtactgttggtaaagccaccatgtgccaccagcagctggtcatcagctggttcagcctggtgttcctggccagccctctggtggccatctgggagctgaagaaagacgtgtacgtggtggaactggactggtatcccgacgcccctggcgagatggtggtgctgacctgcgacacccctgaagaggacggcatcacctggaccctggaccagtctagcgaggtgctgggcagcggcaagaccctgaccatccaggtcaaagagttcggcgacgccggccagtacacctgtcacaagggcggagaggtgctgagccacagcctgctgctgctccacaagaaagaggatggcatttggagcaccgacatcctgaaggaccagaaagagcccaagaacaagaccttcctgagatgcgaggccaagaactacagcggccggttcacctgttggtggctgaccaccatcagcaccgacctgaccttcagcgtgaagtccagcagaggcagcagcgaccctcagggcgtgacatgtggcgccgctacactgtctgccgagagagtgcggggcgacaacaaagagtacgagtacagcgtcgagtgccaggaagatagcgcctgccctgccgccgaggaaagcctgcctatcgaagtgatggtggacgccgtgcacaagctgaagtacgagaactacacctccagctttttcatccgggacatcatcaagcccgaccctcccaagaacctgcagctgaagcctctgaagaacagcagacaggtggaagtgtcctgggagtaccccgacacctggtccacccctcacagctacttcagcctgacattctgtgtgcaagtccagggcaagtccaagcgcgagaaaaaggaccgggtgttcaccgacaagaccagcgccaccgtgatctgccggaagaacgcctctatcagcgtgcgggcccaggaccggtactacagcagctcttggagcgagtgggccagcgtgccatgttctggtggcggaggcggcggaagcagaaatctgccagtggccacccctgaccccggcatgtttccttgtctgcaccacagccagaacctgctgcgggccgtgtccaacatgctgcagaaggcccggcagaccctggaattctacccctgcaccagcgaggaaatcgaccacgaggacatcaccaaggataagaccagcaccgtggaagcctgtctgcccctggaactgaccaagaacgagagctgcctgaacagccgggaaaccagcttcatcaccaacggcagctgtctggccagcagaaagacctccttcatgatggccctgtgcctgagcagcatctacgaggacctgaagatgtaccaggtcgagttcaagaccatgaacgccaagctgctgatggaccccaagcggcagatctttctggaccagaatatgctggccgtgatcgacgagctgatgcaggccctgaacttcaacagcgagacagtgccccagaagtcctccctggaagaacccgacttctacaagaccaagatcaagctgtgcatcctgctgcacgccttccggatcagagccgtgaccatcgacagagtgatgagctacctgaacgcctcctaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagggccaagaggcgtacgtaag

SEQ ID NO: 16 - DNA, 3680 bp, Artificial Sequence
  pASP26
tcgagttaattaagctagcccggtactgttggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttagagggtatataatggaagctcgacttccagcccgggggcattccggtactgttggtaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 17 - DNA, 3689 bp, Artificial Sequence
  pASP28
tcgagttaattaagctagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagaatggccttaccagtgaccgccttgctcctgccgctggccttgctgctccacgccgccaggccgggatccgacatcgttctgagtcagagccccgccatactgagcgcctcccccggggagaaggtgaccatgacctgtagagccagctccagcctttcatttatgcactggtaccagcagaaacccggtagtagtccaaagccgtggatttacgctacatccaacctggcaagcggagtaccagcccgcttctccggcagcggatcaggaacctcctactccctgacaatcagccgcgtcgaagccgaggatgcagccacatacttttgccaccagtggtcctctaatcccctgacatttggcgcaggcaccaagctcgaactgaagcggggatctaccagcggcagtggaaaacctggtagcggagaggggtctaccaaggggcaagtgcaactcagacagcctggcgcagaactcgtgaagcccggcgcatccgtcaagatgtcttgtaaagccagtggctacaccttcactagctataatatgcactgggttaaacagactcccggtcagggcctcgagtggattggcgcaatctacccgggtaatggtgatacttcttacaatcagaagtttaaggggaaagctaccctgaccgccgacaagtcttcatcaaccgcctacatgcagctgagctctctgacgtctgaggattccgccgtgtattactgcgcccggagccactacgggagtaattatgtcgattattttgactactggggtcagggaacaaccctgactgtctccagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 18 - DNA, 3680 bp, Artificial Sequence
  pASP30
tcgagttaattaagctagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 19 - DNA, 3692 bp, Artificial Sequence
  pASP31
tcgagttaattaagctagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 20 - DNA, 4559 bp, Artificial Sequence
  pASP38
tcgagttaattaagctagcccggtactgttggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttagagggtatataatggaagctcgacttccagcccgggggcattccggtactgttggtaaagccaccatgtgccaccagcagctggtcatcagctggttcagcctggtgttcctggccagccctctggtggccatctgggagctgaagaaagacgtgtacgtggtggaactggactggtatcccgacgcccctggcgagatggtggtgctgacctgcgacacccctgaagaggacggcatcacctggaccctggaccagtctagcgaggtgctgggcagcggcaagaccctgaccatccaggtcaaagagttcggcgacgccggccagtacacctgtcacaagggcggagaggtgctgagccacagcctgctgctgctccacaagaaagaggatggcatttggagcaccgacatcctgaaggaccagaaagagcccaagaacaagaccttcctgagatgcgaggccaagaactacagcggccggttcacctgttggtggctgaccaccatcagcaccgacctgaccttcagcgtgaagtccagcagaggcagcagcgaccctcagggcgtgacatgtggcgccgctacactgtctgccgagagagtgcggggcgacaacaaagagtacgagtacagcgtcgagtgccaggaagatagcgcctgccctgccgccgaggaaagcctgcctatcgaagtgatggtggacgccgtgcacaagctgaagtacgagaactacacctccagctttttcatccgggacatcatcaagcccgaccctcccaagaacctgcagctgaagcctctgaagaacagcagacaggtggaagtgtcctgggagtaccccgacacctggtccacccctcacagctacttcagcctgacattctgtgtgcaagtccagggcaagtccaagcgcgagaaaaaggaccgggtgttcaccgacaagaccagcgccaccgtgatctgccggaagaacgcctctatcagcgtgcgggcccaggaccggtactacagcagctcttggagcgagtgggccagcgtgccatgttctggtggcggaggcggcggaagcagaaatctgccagtggccacccctgaccccggcatgtttccttgtctgcaccacagccagaacctgctgcgggccgtgtccaacatgctgcagaaggcccggcagaccctggaattctacccctgcaccagcgaggaaatcgaccacgaggacatcaccaaggataagaccagcaccgtggaagcctgtctgcccctggaactgaccaagaacgagagctgcctgaacagccgggaaaccagcttcatcaccaacggcagctgtctggccagcagaaagacctccttcatgatggccctgtgcctgagcagcatctacgaggacctgaagatgtaccaggtcgagttcaagaccatgaacgccaagctgctgatggaccccaagcggcagatctttctggaccagaatatgctggccgtgatcgacgagctgatgcaggccctgaacttcaacagcgagacagtgccccagaagtcctccctggaagaacccgacttctacaagaccaagatcaagctgtgcatcctgctgcacgccttccggatcagagccgtgaccatcgacagagtgatgagctacctgaacgcctcctaaatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagagccaccatggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 21 - DNA, 4505 bp, Artificial Sequence
  pASP52
tcgagttaattaagctagcttacatatgtctcttggcccgcaggtcctcttcggagatcagcttctgctcgcttcccttgccaggagacaggctcagggacttctgggtgtagtggttgtgcagggcctcgtgcatcacgctgcaggagaacacgtttccctgctgccatctgctcttgtccactgtcagcttgctgtacaggaagaatgagccgtcgctgtccagcacaggaggggttgtcttgtagttgttctcaggctggccattgctctcccattccacggcaatgtcgctggggtaaaagcccttgaccaggcaggtcagggacacctgattcttggtcagctcgtccctgcttggaggcagtgtgtaaacctggggttccctaggctggcccttggccttgctgatggttttctcgataggagcaggcagggccttgttggacaccttgcacttgtactctttgccgttcagccaatcctggtgcagcacggtcagcacggacaccactctgtaggtgctgttgtactgttcctctctaggcttggtcttggcgttgtgcacttccacgccgtccacgtaccaattgaacttcacttcggggtcctcgtgagacacatccaccaccacgcaggtcacttcaggggttctgctgatcatcagggtgtccttaggctttggaggaaacaggaacacggaaggtccgccgagcagttctggagcaggacatggaggacaggtgtgggtcttgtcgctgctcttgggttccactttgatttccaccttggtgccctggccaaatgtgtaaggcagggtgttgccctgctggcagtagtaggtggcaatgtcctcaggctgcaggctagatatggtgaaggtgaagtcggtgccgctgccagagccagaaaatctgctgggcacgccgctgtgcagtctgctggtgtagtagatcagcagtttaggggccttgccgggcttttgctgataccaattcaggtagctgctgatgtcctggctggcccgacaggtgattgtcactctgtctcccacagaggcagacaggctgctggggctctgtgtcatctgaatatcgctaccgcctccgccacttcctccgccaccagatcctccgcctccgctagaaacggtgaccagagagccctggccccaataatccatggcggtggttctggccaggcttctggcgcagtagtacacggctgtatcagcagcggtcacgctagacagccgcaggctgaactggttcttgctggtgtctctcagcatggtcactctggacttcaggctgggattgtaggtggtgatgccgctgtagctgatgtagccgatccattccaggcctcttccaggaggttgtcggacccaggaccaggcatgatcgctggtgatgctgtagccggacacggtacaggtcaggctcagtgtttggctaggccgcacgagtccaggtccagactcttgcagctgaacctcgcactgcacgcctctaagcagagccaccaggaacacccagctcaggccgaattccatggtggctttaccaacagtaccggaatgcccccgggctggaagtcgagcttccattatataccctctaactagtacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccacgccttctgtatgaaacagtttttcctccaacagtaccggatgcatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattctagaatggccttaccagtgaccgccttgctcctgccgctggccttgctgctccacgccgccaggccgggatccgacatcgttctgagtcagagccccgccatactgagcgcctcccccggggagaaggtgaccatgacctgtagagccagctccagcctttcatttatgcactggtaccagcagaaacccggtagtagtccaaagccgtggatttacgctacatccaacctggcaagcggagtaccagcccgcttctccggcagcggatcaggaacctcctactccctgacaatcagccgcgtcgaagccgaggatgcagccacatacttttgccaccagtggtcctctaatcccctgacatttggcgcaggcaccaagctcgaactgaagcggggatctaccagcggcagtggaaaacctggtagcggagaggggtctaccaaggggcaagtgcaactcagacagcctggcgcagaactcgtgaagcccggcgcatccgtcaagatgtcttgtaaagccagtggctacaccttcactagctataatatgcactgggttaaacagactcccggtcagggcctcgagtggattggcgcaatctacccgggtaatggtgatacttcttacaatcagaagtttaaggggaaagctaccctgaccgccgacaagtcttcatcaaccgcctacatgcagctgagctctctgacgtctgaggattccgccgtgtattactgcgcccggagccactacgggagtaattatgtcgattattttgactactggggtcagggaacaaccctgactgtctccagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 22 - protein, 509 aa, Artificial Sequence
  anti-hIL-6R scFv-Fc
MEFGLSWVFLVALLRGVQCEVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSEQKLISEEDLRAKR

SEQ ID NO: 23 - protein, 495 aa, Artificial Sequence
  Anti-CD20 CAR human
MALPVTALLLPLALLLHAARPGSDIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKRGSTSGSGKPGSGEGSTKGQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTTLTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 495 aa, Artificial Sequence
  Anti-CD20 CAR canine
MASRVTALLLPLALLLRAAAASDIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKRGSTSGSGKPGSGEGSTKGQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTTLTVSSASPTTPAPRPPTRAPTNASKPVSPRGETCRPAAGSAVKTSGLDFACEIYIWAPLAGTCAVLLLSLVITIICHGRKKLLYLFKQPFMRPVQTAQEEDACSCRFPEEEEGECDLRAKFGRSAAAPEHQQGPNQLYNELNLRGREEYEVLDKRRGLDPEMGGKQRKRNPQEVVYNALQKDKMAEAYSEIGIKSENQRRRGKGHDGLYQGLSTATKDTYDALHMQALPPR

SEQ ID NO: 25 - protein, 490 aa, Artificial Sequence
  Anti-Her2 human CAR with 4-1BB costimulatory domain
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 494 aa, Artificial Sequence
  Anti-Her2 CAR with CD28 costimulatory domain
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 532 aa, Artificial Sequence
  hIL-12 (p40-linker-p35)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 533 aa, Artificial Sequence
  cIL-12 (p40-linker-p35)
MHPQQLVISWFSLVLLASSLMTIWELEKDVYVVELDWHPDAPGEMVVLTCHTPEEDDITWTSAQSSEVLGSGKTLTIQVKEFGDAGQYTCHKGGKVLSRSLLLIHKKEDGIWSTDILKEQKESKNKIFLKCEAKNYSGRFTCWWLTAISTDLKFSVKSSRGFSDPQGVTCGAVTLSAERVRVDNRDYKKYTVECQEGSACPSAEESLPIEVVVDAIHKLKYENYTSSFFIRDIIKPDPPTNLQLKPLKNSRHVEVSWEYPDTWSTPHSYFSLTFCVQAQGKNNREKKDRLCVDKTSAKVVCHKDAKIRVQARDRYYSSSWSDWASVSCSGGGGGGSRSLPTASPSPGIFQCLNHSQNLLRAVSNTLQKARQTLELYSCTSEEIDHEDITKDKTSTVEACLPLELTMNESCLASREISLITNGSCLASGKASFMTVLCLSSIYEDLKMYQMEFKAMNAKLLMDPKRQIFLDQNMLTAIDELLQALNFNSVTVPQKSSLEEPDFYKTKIKLCILLHAFRIRAVTIDRMMSYLNSS

SEQ ID NO: 29 - DNA, 23 bp, Artificial Sequence
  Spacer
ggcattccggtactgttggtaaa

SEQ ID NO: 30 - protein, 384 aa, Homo Sapiens
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTETNWPRELKDGNGQESLSMSSSSSPA

SEQ ID NO: 31 - protein, 655 aa, Homo Sapiens
MAEAPQVVEIDPDFEPLPRPRSCAWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAAAMDNNSKFAKSRSRAAKKKASLQSGQEGAGDSPGSQFSKWPASPGSHSNDDFDNWSTFRPRTSANASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 32 - DNA, 111 bp, Artificial Sequence
  STAT3 responsive element
ccggtactgttagcttcatttcccgtaaatcgtcgaagcttcatttcccgtaaatcgtcgaagcttcatttcccgtaaatcgtcgaagcttcatttcccgtaaatcgtcga

SEQ ID NO: 33 - DNA, 1404 bp, Artificial Sequence
  hIL-6Ra
atgctggccgtgggatgtgctttgcttgctgctctgttggctgctcctggtgctgcactggcccctagaagatgtcctgctcaagaggtggcaagaggcgtgctgacatctctgcctggcgatagcgtgaccctgacatgtcctggcgtggaacccgaggataatgccaccgtgcattgggtgctgagaaagcctgccgccggatctcatcctagcagatgggctggcatgggccgtagactgctgctgagatctgtccagctgcacgacagcggcaactacagctgttacagagccggcagacctgccggaacagtgcatctgctggtggatgtgcctcctgaggaaccccagctgagctgcttcagaaagtcccctctgagcaacgtcgtgtgcgagtggggccctagaagcacacctagcctgacaacaaaggccgtgctgctcgtgcggaagttccagaattctcccgccgaggacttccaagagccttgccagtacagccaagagagccagaagttcagctgtcagctggctgtgcctgagggcgacagcagcttctacatcgtgtctatgtgcgtggccagcagcgtgggcagcaagtttagcaagacccagaccttccaaggctgcggcatcctgcaacctgatcctccagccaacatcaccgtgaccgccgtggccagaaatcccagatggctgtctgtgacctggcaggaccctcacagctggaactccagcttttaccggctgagattcgagctgcggtacagggccgagagaagcaagaccttcaccacctggatggtcaaggacctgcagcatcactgcgtgatccacgatgcttggagcggcctgagacatgtggtgcagctgagagcccaagaggaattcggccaaggcgagtggagtgaatggtcccctgaagccatgggcaccccttggacagagagcagatctccaccagccgagaacgaggtgtccacacctatgcaggccctgaccaccaacaaggacgacgacaacatcctgttccgggacagcgccaatgccacaagtctgcctgtgcaggatagctcctctgtgcccctgcctacctttctggttgctggcggatctctggcctttggcaccctgctgtgtatcgccatcgtgctgcggttcaagaaaacctggaagctgcgggccctgaaagagggcaagacctctatgcaccctccttacagcctgggccagctggtgcctgaaagacctagacctacacctgtgctggtgcccctgatcagccctcctgtgtctccatctagcctgggcagcgacaataccagcagccacaacagacccgacgctcgggaccctagatctccctacgacatcagcaataccgactacttcttcccacgc

SEQ ID NO: 34 - DNA, 1422 bp, Artificial Sequence
  Regulatory unit
aggctggatcggtcccggtgtcttctatggaggtcaaaacagcgtggatggcgtctccaggcgatctgacggttcactaaacgagctctgcttatataggcctcccaccgtacacgcctacggaggacagtactccgtgcggaggacagtactccgtccggaggacagtactccgatcggaggacagtactccggtcggaggacagtactccgctcggaggacagtactccgacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 35 - protein, 236 aa, Artificial Sequence
  mCherry reporter
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 36 - protein, 781 aa, Artificial Sequence
  4D5-Notch1-Gal4-VP64 receptor
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 37 - protein, 233 aa, Artificial Sequence
  BFP2 reporter
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN